SEQUENCE LISTING (42 sequences)

SEQ ID NO: 1 - DNA, 11 bp, Artificial Sequence
  Description of Artificial Sequence Synthetic oligonucleotide
tgatatcacag

SEQ ID NO: 2 - DNA, 11 bp, Artificial Sequence
  Description of Artificial Sequence Synthetic oligonucleotide
accattccttc

SEQ ID NO: 3 - DNA, 21 bp, Artificial Sequence
  Description of Artificial Sequence Synthetic oligonucleotide
ggcaaggcaaggcaaggcaca

SEQ ID NO: 4 - DNA, 19 bp, Artificial Sequence
  Description of Artificial Sequence Synthetic oligonucleotide
gctcttatggtgagagtgt

SEQ ID NO: 5 - DNA, 18 bp, Artificial Sequence
  Description of Artificial Sequence Synthetic oligonucleotide
gttagggttagggttagg

SEQ ID NO: 6 - DNA, 17 bp, Artificial Sequence
  Description of Artificial Sequence Synthetic oligonucleotide
gctcacaattccacatg

SEQ ID NO: 7 - DNA, 20 bp, Artificial Sequence
  Description of Artificial Sequence Synthetic oligonucleotide
gccacatgtggaattgtgag

SEQ ID NO: 8 - DNA, 32 bp, Artificial Sequence
  Description of Artificial Sequence Synthetic oligonucleotide
gtacgttctctatcactgatatgatatcacag

SEQ ID NO: 9 - DNA, 21 bp, Artificial Sequence
  Description of Artificial Sequence Synthetic oligonucleotide
gatcagctctctcacggtgac

SEQ ID NO: 10 - DNA, 21 bp, Artificial Sequence
  Description of Artificial Sequence Synthetic oligonucleotide
gcacttggctgagtccacagt

SEQ ID NO: 11 - DNA, 21 bp, Artificial Sequence
  Description of Artificial Sequence Synthetic oligonucleotide
gacatcggagaatgcacgctc

SEQ ID NO: 12 - DNA, 20 bp, Artificial Sequence
  Description of Artificial Sequence Synthetic oligonucleotide
gaataggtcgatgtagagca

SEQ ID NO: 13 - DNA, 21 bp, Artificial Sequence
  Description of Artificial Sequence Synthetic oligonucleotide
gccgcgttctgtaagaatcgg

SEQ ID NO: 14 - DNA, 20 bp, Artificial Sequence
  Description of Artificial Sequence Synthetic oligonucleotide
gtaagtcctatgacagaagc

SEQ ID NO: 15 - DNA, 21 bp, Artificial Sequence
  Description of Artificial Sequence Synthetic oligonucleotide
gagatatactgttagcgcctt

SEQ ID NO: 16 - DNA, 20 bp, Artificial Sequence
  Description of Artificial Sequence Synthetic oligonucleotide
ggctgtagacatcaatgctt

SEQ ID NO: 17 - DNA, 21 bp, Artificial Sequence
  Description of Artificial Sequence Synthetic oligonucleotide
gatcattgcaggtaagaagtg

SEQ ID NO: 18 - DNA, 21 bp, Artificial Sequence
  Description of Artificial Sequence Synthetic oligonucleotide
gctccaacactctaccttgta

SEQ ID NO: 19 - DNA, 20 bp, Artificial Sequence
  Description of Artificial Sequence Synthetic oligonucleotide
ggaagtgcttacgagtcaat

SEQ ID NO: 20 - DNA, 21 bp, Artificial Sequence
  Description of Artificial Sequence Synthetic oligonucleotide
gctctgtcagtaactgataag

SEQ ID NO: 21 - DNA, 20 bp, Artificial Sequence
  Description of Artificial Sequence Synthetic oligonucleotide
gcaagcttacctgatagatt

SEQ ID NO: 22 - DNA, 21 bp, Artificial Sequence
  Description of Artificial Sequence Synthetic oligonucleotide
gttccatcttctaagtgtcct

SEQ ID NO: 23 - DNA, 20 bp, Artificial Sequence
  Description of Artificial Sequence Synthetic oligonucleotide
gaactgagacttgtgacaca

SEQ ID NO: 24 - DNA, 20 bp, Artificial Sequence
  Description of Artificial Sequence Synthetic oligonucleotide
gagttagaagtcttaagacc

SEQ ID NO: 25 - DNA, 21 bp, Artificial Sequence
  Description of Artificial Sequence Synthetic oligonucleotide
gttccatccaactcaggcctt

SEQ ID NO: 26 - DNA, 21 bp, Artificial Sequence
  Description of Artificial Sequence Synthetic oligonucleotide
gcctgaggcttctatctatct

SEQ ID NO: 27 - DNA, 20 bp, Artificial Sequence
  Description of Artificial Sequence Synthetic oligonucleotide
ggaaggctcatatggataga

SEQ ID NO: 28 - DNA, 20 bp, Artificial Sequence
  Description of Artificial Sequence Synthetic oligonucleotide
gatcatctcacatggcagcc

SEQ ID NO: 29 - DNA, 20 bp, Artificial Sequence
  Description of Artificial Sequence Synthetic oligonucleotide
ggctatcagagcaagcattg

SEQ ID NO: 30 - DNA, 20 bp, Artificial Sequence
  Description of Artificial Sequence Synthetic oligonucleotide
gtgtgtgtcatgtgtggcag

SEQ ID NO: 31 - DNA, 20 bp, Artificial Sequence
  Description of Artificial Sequence Synthetic oligonucleotide
gcgcatgcgccgctggggcg

SEQ ID NO: 32 - DNA, 23 bp, Artificial Sequence
  Description of Artificial Sequence Synthetic oligonucleotide
gcagacgcgaggaaggagggcgc

SEQ ID NO: 33 - DNA, 19 bp, Artificial Sequence
  Description of Artificial Sequence Synthetic oligonucleotide
ggtagcaaagtgacgccga

SEQ ID NO: 34 - DNA, 20 bp, Artificial Sequence
  Description of Artificial Sequence Synthetic oligonucleotide
gctccagtagccaccgcatc

SEQ ID NO: 35 - DNA, 20 bp, Artificial Sequence
  Description of Artificial Sequence Synthetic oligonucleotide
gcctatatagtgcgggtggg

SEQ ID NO: 36 - DNA, 20 bp, Artificial Sequence
  Description of Artificial Sequence Synthetic oligonucleotide
gtgagtcgaggagaaacgac

SEQ ID NO: 37 - DNA, 3 bp, Homo sapiens
tgg

SEQ ID NO: 38 - DNA, 20 bp, Artificial Sequence
  Description of Artificial Sequence Synthetic oligonucleotide
gcgacggggggagtgaggag

SEQ ID NO: 39 - DNA, 3 bp, Homo sapiens
ggg

SEQ ID NO: 40 - DNA, 36 bp, Artificial Sequence
  Description of Artificial Sequence Synthetic probe
ttgttatccgctcacaattccacatgtggccacaaa

SEQ ID NO: 41 - DNA, 22 bp, Artificial Sequence
  Description of Artificial Sequence Synthetic probe
cccacactctcaccataagagc

SEQ ID NO: 42 - DNA, 21 bp, Artificial Sequence
  Description of Artificial Sequence Synthetic probe
ttgccttgtgccttgccttgc

(19) United States
(12) Patent Application Publication
(10) Pub. No.: US 2022/0056097 A1
(43) Pub. Date: Feb. 24, 2022

(54) SYSTEMS AND METHODS FOR COMPARTMENT SPATIAL ORGANIZATION

(71) Applicant: The Board of Trustees of the Leland Stanford Junior University, Stanford, CA (US)

(72) Inventors: Lei S. Qi, Stanford, CA (US); Yuchen Gao, Menlo Park, CA (US)

(73) Assignee: The Board of Trustees of the Leland Stanford Junior University, Stanford, CA (US)

(21) Appl. No.: 17/222,851

(22) Filed: Apr. 5, 2021

Related U.S. Application Data
(63) Continuation of application No. PCT/US2019/055976, filed on Oct. 11, 2019.
(60) Provisional application No. 62/744,504, filed on Oct. 11, 2018.

(51) Int. Cl. C07K 14/575; C12N 9/22; C12N 15/11; C12N 15/90

(52) U.S. Cl. CPC C07K 14/575; C12N 9/22; C12N 15/11; C12N 15/907; C07K 2319/09; C12N 2310/20; C07K 2319/00; C07K 2319/75; C12N 2800/80

(57) ABSTRACT

Provided herein are systems and methods for the controlling the spatial positioning of a compartment or compartment-like environment to a target polynucleotide in a cell.

### CROSS-REFERENCE TO RELATED APPLICATIONS

The present application is a continuation of International Application No. PCT/US2019/055976, filed Oct. 11, 2019, which claims priority to and benefit from U.S. Provisional Application No. 62/744,504, filed Oct. 11, 2018, the entire contents of which are herein incorporated by reference.

### SEQUENCE LISTING

The instant application contains a Sequence Listing which has been submitted electronically in ASCII format and is hereby incorporated by reference in its entirety. Said ASCII copy, created on Apr. 5, 2021, is named 079445-003410US-1238452_SL.txt and is 9,957 bytes in size.

### BACKGROUND

The 3-dimensional (3D) spatial organization of polynucleotides within living cells plays an important role in such processes as regulating and maintaining gene expression, genome stability, and cellular function.

There exists a need for systems and methods to carry out the spatial organization of target polynucleotides. The present disclosure addresses this and other needs.

### SUMMARY

In general, provided herein are systems and methods for programmable polynucleotide re-organization. The systems and methods can couple an actuator moiety with cellular compartment-specific proteins via a chemically inducible system, and can allow efficient, inducible, and dynamic repositioning of polynucleotides, e.g., genomic loci, to particular cellular positions, e.g., the nuclear periphery, Cajal bodies, and PML nuclear bodies. (FIG. 1) For example, the systems and methods can couple an actuator moiety with cellular compartment-specific proteins via a chemically inducible system, and can allow efficient, inducible, and dynamic formation of the compartment of the compartment-specific proteins, e.g., the nuclear heterochromatin, Cajal bodies, and PML nuclear bodies, around the polynucleotides, e.g., genomic loci. The systems and methods can expand existing polynucleotide editing and regulation tools, offering an improved technology to manipulate the 3D organization of polynucleotides relative to cellular compartments, to manipulate cellular compartments around the 3D organization of polynucleotides, and to study the relationship between macro-scale spatial polynucleotide organization and cellular function. Furthermore, the formation of compartments around a target polynucleotide can aid in manipulation of a cell's fate or function.

In some aspects, a composition comprises: a) a compartment-constituent protein linked to a first dimerization domain; and b) an actuator moiety linked to a scaffold, wherein the scaffold is linked to a second dimerization domain; and wherein the first dimerization domain binds to the second dimerization domain. In some embodiments, the scaffold is linked to at least 1, 2, 3, 4, 5, 6, 7, 8, 9, 10 or more second dimerization domains. In some embodiments, the compartment-constituent protein is further linked to a second scaffold, wherein the second scaffold is linked to at least one first dimerization domain. In some embodiments, the second scaffold is linked to at least 1, 2, 3, 4, 5, 6, 7, 8, 9, 10, or more first dimerization domains. In some embodiments, the scaffold or the second scaffold is a repeating peptide array. In some embodiments, the scaffold or the second scaffold is a SpyTag, SunTag, split sfGFP, MoonTag, SnoopTag, split sfCherry2, or mNeonGreen2 protein scaffold. In some embodiments, the composition further comprises a ligand, wherein the binding of the first dimerization domain to the second dimerization domain occurs in the presence of the ligand. The composition of any one of claims 1-6, wherein the composition regulates an expression of the target polynucleotide. In some embodiments, the composition increases an expression of the target polynucleotide. In some embodiments, the composition decreases an expression of the target polynucleotide. In some embodiments, the composition regulates an expression of an additional polynucleotide. In some embodiments, the composition increases an expression of an additional polynucleotide. In some embodiments, the composition decreases an expression of an additional polynucleotide. In some embodiments, the additional polynucleotide is a distal gene to the target polynucleotide. In some embodiments, the additional polynucleotide is a proximal gene to the target polynucleotide. In some embodiments, a three-dimensional structure of the complex regulates a gene or a regulatory element of a gene. In some embodiments, the regulatory element is an enhancer or a promoter. In some embodiments, the three-dimensional structure forms a three-dimensional loop, a chromosome boundary, a topologically associating domain, or a gene cluster. In some embodiments, the three-dimensional loop is formed between an enhancer and a promoter. In some embodiments, the composition insulates a target polynucleotide. In some embodiments, the composition insulates a target polynucleotide by separating the target polynucleotide for chromosome protection or manipulation. In some embodiments, the composition traps a target polynucleotide in a spatial region of a compartment. In some embodiments, the spatial region of a compartment manipulates the fate or function of the target polynucleotide or the additional polynucleotide. In some embodiments, the spatial region of a compartment promotes or prevents recombination or mutagenesis, promotes or inhibits gene expression, splicing or translation, promotes or inhibits polynucleotide transport or movement In some embodiments, the composition introduces epigenetic modifications at the target polynucleotide. In some embodiments, the epigenetic modification is DNA methylation, DNA demethylation, histone methylation, histone demethylation, acetylation, deacetylation, phosphorylation, dephosphorylation, ubiquitylation, GlcNAcylation, citrullination, krotonilation, isomerization, or any combination thereof. In some embodiments, the composition repairs a DNA break. In some embodiments, the composition repairs a DNA break by introducing exogenous DNA. In some embodiments, the composition repairs a DNA break by introducing recombination, non-homologous end-joining, or homology-directed repair. In some embodiments, the composition creates an artificial aggregate, wherein the artificial aggregate comprises protein, RNA, DNA, or a combination thereof. In some embodiments, the artificial aggregate proteins, RNAs, DNAs, or a combination thereof are recruited by the compartment-constituent protein. In some embodiments, the target polynucleotide is genomic DNA. In some embodiments, the target polynucleotide is a polynucleotide encoding a gene. In some embodiments, the target polynucleotide is a non-coding polynucleotide. In some embodiments, the target polynucleotide is a tandem repeat region of genomic DNA. In some embodiments, the target polynucleotide is RNA. In some embodiments, the compartment is a Cajal body. In some embodiments, the compartment-constituent protein comprises a protein from a Cajal body. In some embodiments, the compartment-constituent protein comprises coilin, SMN, Gemin 3, SmD1, SmE, or a combination thereof. In some embodiments, the compartment is a nuclear speckle. In some embodiments, the compartment-constituent protein is a protein from a nuclear speckle. In some embodiments, the compartment-constituent protein comprises SC35. In some embodiments, the compartment is a PML body. In some embodiments, the compartment-constituent protein is a protein from a PML body. In some embodiments, the compartment-constituent protein comprises PML, SP100, or a combination thereof. In some embodiments, the compartment is a cytosolic compartment. In some embodiments, the compartment-constituent protein is a protein from a cytosolic compartment. In some embodiments, the compartment is a synthetic cellular phase. In some embodiments, the compartment-constituent protein is a protein from a synthetic cellular phase. In some embodiments, the compartment-constituent protein comprises Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. In some embodiments, the synthetic cellular phase facilitates homology directed repair. In some embodiments, the compartment is nuclear heterochromatin. In some embodiments, the compartment-constituent protein is a protein from nuclear heterochromatin. In some embodiments, the compartment-constituent protein comprises HP1α, HP1β, KAP1, KRAB, SUV39H1, or G9a. In some embodiments, the compartment-constituent protein further comprises an oligomerization domain. In some embodiments, the compartment-constituent protein is further linked to a fluorescent protein. In some embodiments, the actuator moiety comprises a binding protein that hybridizes to the target polynucleotide. In some embodiments, the actuator moiety comprises a Cas protein, and wherein the system further comprises: (c) a guide RNA that complexes with the actuator moiety and hybridizes to the target polynucleotide. In some embodiments, the actuator moiety comprises an RNA binding protein complexed with a guide RNA that hybridizes to the target polynucleotide, and wherein the composition further comprises: (c) a Cas protein that complexes with the guide RNA. In some embodiments, the Cas protein substantially lacks DNA cleavage activity. In some embodiments, the Cas protein is a Cas9 protein, a Cas12 protein, a Cas13 protein, a CasX protein, or a CasY protein. In some embodiments, the Cas12 protein is selected from the group consisting of Cas12a, Cas12b, Cas12c, Cas12d, and Cas12e. In some embodiments, the actuator moiety comprises a binding protein that hybridizes to the target polynucleotide, wherein the binding protein is a zinc finger nuclease or a TALE nuclease. In some embodiments, the actuator moiety comprises an Argonaute protein complexed with a guide polynucleotide, wherein the guide polynucleotide is a guide RNA or a guide DNA, and wherein the guide polynucleotide hybridizes to the target polynucleotide. In some embodiments, the actuator moiety is further linked to a fluorescent protein. In some embodiments, the first dimerization domain binds to the second dimerization domain in the presence of a ligand. In some embodiments, the first dimerization domain binds to the ligand and the ligand binds to the second dimerization domain to assemble a dimer. In some embodiments, the ligand is a chemically inducible. In some embodiments, the ligand is abscisic acid. In some embodiments, the dimer is inducible and reversible. In some embodiments, the dimer is a heterodimer. In some embodiments, the dimer is a homodimer. In some embodiments, the first dimerization domain is an ABI domain. In some embodiments, the second dimerization domain is a PYL1 domain. In some embodiments, the first dimerization domain is a PYL1 domain. In some embodiments, the second dimerization domain is an ABI domain. In some embodiments, the compartment-constituent protein linked to the first dimerization domain is a fusion protein. In some embodiments, the actuator moiety linked to the second dimerization domain is a fusion protein. In some embodiments, the compartment-constituent protein linked to the first dimerization domain by a linker. In some embodiments, the actuator moiety linked to the second dimerization domain by a linker.

In some aspects, the method comprises: (a) providing a compartment-constituent protein linked to a first dimerization domain; (b) providing an actuator moiety linked to a second dimerization domain, wherein the actuator moiety and the target polynucleotide form a complex; and (c) assembling a dimer comprising the first dimerization domain and the second dimerization domain of the complex, thereby forming the compartment around the target polynucleotide. In some embodiments, the method further comprises providing a ligand before step (c), wherein the ligand binds the first dimerization domain to the second dimerization domain for assembling the dimer of step (c). In some embodiments, the method further comprises regulating an expression of the target polynucleotide after the formation of the compartment around the target polynucleotide. In some embodiments, the method further comprises increasing an expression of the target polynucleotide after the formation of the compartment around the target polynucleotide compared to before the formation of the compartment around the target polynucleotide. In some embodiments, the method further comprises decreasing an expression of the target polynucleotide after the formation of the compartment around the target polynucleotide compared to before the formation of the compartment around the target polynucleotide. In some embodiments, the method further comprises regulating an expression of an additional polynucleotide after the formation of the compartment around the target polynucleotide. In some embodiments, the method further comprises increasing an expression of an additional polynucleotide after the formation of the compartment around the target polynucleotide compared to before the formation of the compartment around the target polynucleotide. In some embodiments, the method further comprises decreasing an expression of an additional polynucleotide after the formation of the compartment around the target polynucleotide compared to before the formation of the compartment around the target polynucleotide. In some embodiments, the additional polynucleotide is a distal gene to the target polynucleotide. In some embodiments, the additional polynucleotide is a proximal gene to the target polynucleotide. In some embodiments, a three-dimensional structure of the complex regulates a gene or a regulatory element of a gene. In some embodiments, the regulatory element is an enhancer or a promoter. In some embodiments, the three-dimensional structure forms a three-dimensional loop, a chromosome boundary, a topologically associating domain, or a gene cluster. In some embodiments, the three-dimensional loop is formed between an enhancer and a promoter. In some embodiments, the method further comprises insulating a target polynucleotide. In some embodiments, the insulating comprises separating the target polynucleotide for chromosome protection or manipulation. In some embodiments, the method further comprises trapping a target polynucleotide in a spatial region of a compartment. In some embodiments, the method further comprises manipulating the fate or function of the target polynucleotide or the additional polynucleotide in the spatial region or compartment. In some embodiments, the spatial region of a compartment promotes or prevents recombination or mutagenesis, promotes or inhibits gene expression, splicing or translation, promotes or inhibits polynucleotide transport or movement. In some embodiments, the method further comprises introducing epigenetic modifications at the target polynucleotide after the forming of the compartment around the target polynucleotide.

The method of any one of claim 100, wherein the epigenetic modification is DNA methylation, DNA demethylation, histone methylation, histone demethylation, acetylation, deacetylation, phosphorylation, dephosphorylation, ubiquitylation, GlcNAcylation, citrullination, krotonilation, isomerization, or any combination thereof. In some embodiments, the method further comprises repairing a DNA break. In some embodiments, the repairing comprises introducing exogenous DNA. In some embodiments, the introducing comprises recombination, non-homologous end-joining, or homology-directed repair. In some embodiments, the formation of the compartment around the target polynucleotide further comprises creating an artificial aggregate, wherein the artificial aggregate comprises protein, RNA, DNA, or a combination thereof. In some embodiments, the artificial aggregate proteins, RNAs, DNAs, or a combination thereof are recruited by the compartment-constituent protein. In some embodiments, the target polynucleotide is genomic DNA. In some embodiments, the target polynucleotide is polynucleotide encoding a gene. In some embodiments, the target polynucleotide is noncoding polynucleotide. In some embodiments, the target polynucleotide is a tandem repeat region of genomic DNA. In some embodiments, the target polynucleotide is RNA. In some embodiments, the compartment is a Cajal body. In some embodiments, the compartment-constituent protein comprises a protein from a Cajal body. In some embodiments, the compartment-constituent protein comprises coilin, SMN, Gemin 3, SmD1, SmE, or a combination thereof. In some embodiments, the compartment is a nuclear speckle. In some embodiments, the compartment-constituent protein is a protein from a nuclear speckle. In some embodiments, the compartment-constituent protein comprises SC35. In some embodiments, the compartment is a PML body. In some embodiments, the compartment-constituent protein is a protein from a PML body. In some embodiments, the compartment-constituent protein comprises PML, SP100, or a combination thereof. In some embodiments, the compartment is a cytosolic compartment. In some embodiments, the compartment-constituent protein is a protein from a cytosolic compartment. In some embodiments, the compartment is a synthetic cellular phase. In some embodiments, the compartment-constituent protein is a protein from a synthetic cellular phase. In some embodiments, the compartment-constituent protein comprises Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. In some embodiments, the synthetic cellular phase facilitates homology directed repair. In some embodiments, the compartment is nuclear heterochromatin. In some embodiments, the compartment-constituent protein is a protein from nuclear heterochromatin. In some embodiments, the compartment-constituent protein comprises HP1α, HP1β, KAP1, KRAB, SUV39H1, or G9a. In some embodiments, the compartment-constituent protein further comprises an oligomerization domain. In some embodiments, the compartment-constituent protein is further linked to a fluorescent protein. In some embodiments, the actuator moiety comprises a binding protein that hybridizes to the target polynucleotide. In some embodiments, the actuator moiety comprises a Cas protein, and wherein the system further comprises: (c) a guide RNA that complexes with the actuator moiety and hybridizes to the target polynucleotide. In some embodiments, the actuator moiety comprises an RNA binding protein complexed with a guide RNA that hybridizes to the target polynucleotide, and wherein the system further comprises: (c) a Cas protein that complexes with the guide RNA. In some embodiments, the Cas protein substantially lacks DNA cleavage activity. In some embodiments, the Cas protein is a Cas9 protein, a Cas12 protein, a Cas13 protein, a CasX protein, or a CasY protein. In some embodiments, the Cas12 protein is selected from the group consisting of Cas12a, Cas12b, Cas12c, Cas12d, and Cas12e. In some embodiments, the actuator moiety comprises a binding protein that hybridizes to the target polynucleotide, wherein the binding protein is a zinc finger nuclease or a TALE nuclease. In some embodiments, the actuator moiety comprises an Argonaute protein complexed with a guide polynucleotide, wherein the guide polynucleotide is a guide RNA or a guide DNA, and wherein the guide polynucleotide hybridizes to the target polynucleotide. In some embodiments, the actuator moiety is further linked to a fluorescent protein. In some embodiments, the actuator moiety is further linked to a scaffold, wherein the actuator is linked to the scaffold and the scaffold is linked to at least one second dimerization domains In some embodiments, the actuator moiety is further linked to a scaffold, wherein the actuator is linked to the scaffold and the scaffold is linked to 2, 3, 4, 5, 6, 7, 8, 9, 10, or more second dimerization domains. In some embodiments, the scaffold is a repeating peptide array. In some embodiments, the scaffold is a SpyTag, SunTag, split sfGFP, MoonTag, SnoopTag, split sfCherry2, or mNeonGreen2 protein scaffold. In some embodiments, the assembling occurs in the presence of a ligand. In some embodiments, the first dimerization domain binds to the ligand and ligand binds to the second dimerization domain to assemble the dimer. In some embodiments, the ligand is a chemically inducible In some embodiments, the ligand is abscisic acid. In some embodiments, the assembling is inducible and reversible. In some embodiments, the dimer is a heterodimer. In some embodiments, the dimer is a homodimer. In some embodiments, the first dimerization domain is an ABI domain. In some embodiments, the second dimerization domain is a PYL1 domain. In some embodiments, the first dimerization domain is a PYL1 domain. In some embodiments, the second dimerization domain is a ABI domain. In some embodiments, the compartment-constituent protein linked to the first dimerization domain is a fusion protein. In some embodiments, the actuator moiety linked to the second dimerization domain is a fusion protein. In some embodiments, the compartment-constituent protein is linked to the first dimerization domain by a linker. In some embodiments, the actuator moiety is linked to the second dimerization domain by a linker.

In some aspects, a method of treating a disease in a subject in need thereof, by administering the composition of any one of the previous embodiments. In some embodiments, the disease is a protein misfolding disease. In some embodiments, the disease is selected from the group consisting of Alzheimer's disease (AD), Parkinson's disease (PD), multisystem atrophy, Huntington's disease (HD), prion diseases, Amyotrophic lateral sclerosis (ALS) and frontotemporal dementia (FTD).

In some aspects, a system comprises the composition of any one of the preceding embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 is a schematic illustration of a programmable, inducible, and versatile system for targeting genomic loci to various nuclear compartments. dCas9 and a nuclear compartment-specific protein are fused to complementary pairs of heterodimerization domains, which assemble only in the presence of a chemical inducer. The genomic targets are specified by the sgRNA sequences, and nuclear compartments are programmed by fusing CRISPR-GO with compartment-specific molecules.

FIG. 2 is a schematic illustration of an abscisic acid (ABA)-inducible CRISPR-GO system to target genomic loci to the nuclear envelope (NE) through co-expression of ABI-dCas9 and PYL1-GFP-Emerin in human cells. In the presence of ABA, ABI and PYL1 dimerize, causing relocalization of ABI-dCas9-targeted genomic loci to PYL1-GFP-Emerin at the nuclear envelope. After removal of ABA, ABI and PYL1 dissociate and genomic loci are no longer tethered to the NE.

FIG. 3 is a schematic illustration of the ABA-inducible CRISPR-GO system with co-expression of ABI-BFP-dCas9 and PYL1-GFP-Emerin in human cells. ABA treatment dimerizes ABI and PYL1 and re-localizes ABI-BFP-dCas9-targeted genomic loci to the nuclear periphery containing PYL1-GFP-Emerin.

FIG. 4 is a schematic illustration of the TMP-HTag inducible CRISPR-GO system with co-expression of dCas9-EGFP-HaloTag and DHFR-Emerin-mCherry in human cells. TMP-HTag treatment dimerizes DHFR and HaloTag and re-localizes dCas9-EGFP-HaloTag-targeted genomic loci to the nuclear periphery containing DHFR-Emerin-mCherry.

FIG. 5 is a schematic illustration of the method to use CRISPR-Cas9 imaging to visualize repetitive genomic loci targeted by the CRISPR-GO system in living cells. Both ABI-dCas9 and dCas9-HaloTag bind to the same repetitive genomic locus. While ABI-dCas9 dimerizes with PYL1-Emerin to re-localize the genomic locus, dCas9-HaloTag binds to cell permeable JF549-HaloTag dye ligand to enable visualization of the targeted genomic locus in living cells.

FIG. 6 presents representative microscopic images of U2OS cells showing co-expression of ABI-BFP-dCas9, PYL1-GFP-Emerin, and dCas9-HaloTag, without sgRNAs. ABI-BFP-dCas9 likely accumulate in nucleoli without ABA treatment. ABA treatment-induced heterodimerization relocated ABI-BFP-dCas9 to the nuclear envelope (NE) and Endoplasmic Reticulum (ER), as marked by PYL1-GFP-Emerin. dCas9-HaloTag had a low expression level and was evenly distributed throughout the nucleus; its location remained unaffected by ABA treatment. Scale bars, 10 μm.

FIG. 7 is a summary of chromosome locations of highly repetitive regions targeted by CRISPR-GO in FIG. 8 and FIG. 9. A single sgRNA binds to multiple repeats (solid grey boxes) within the targeted regions. The genes adjacent to the targeted site are shown in italic letters in grey-outlined boxes.

FIG. 8 presents graphs of the quantification of CRISPR-GO-induced genomic repositioning efficiency of highly repetitive genomic loci. Chr3, Chr13, and LacO loci are labeled using CRISPR-Cas9 imaging in living cells. Telomeres are labeled by a telomere marker, TRF1-mCherry. The nuclear envelope is visualized by GFP-Emerin. The numbers of loci and cells analyzed are on the bottom. From left to right, graphs 1 (top), 1 (bottom), 3 (top), and 3 (bottom), show the percent of loci at the nuclear periphery, and graphs 2 (top), 2 (bottom), 4 (top), and 4 (bottom), show percentage of cells containing at least one periphery-associated locus.

FIG. 9 presents graphs of the quantification of CRISPR-GO-induced nuclear repositioning efficiency of less repetitive endogenous genomic loci. Genomic loci were visualized by 3D-FISH and nuclei are stained by DAPI. From left to right: Graph 1 and Graph 3: the percentage of genomic loci at the nuclear periphery; Graph 2 and Graph 4: the percentage of cells containing at least one nuclear periphery-associated locus. The numbers of loci and cells analyzed are on the bottom.

FIG. 10 presents representative microscopy images comparing the localization of targeted genomic loci (arrows) labeled by CRISPR-Cas9 imaging with or without ABA. PYL1-GFP-Emerin is shown localized to the nuclear envelope (NE) and endoplasmic reticulum (ER). The nuclear periphery is outlined by dotted white lines except for regions next to tethered genomic loci. Insets show enlarged images of periphery-tethered genomic loci. Scale bars, 10 μm.

FIG. 11 presents individual channels of the representative microscopic images in FIG. 10 comparing the localization of targeted genomic loci (arrows) and nuclear periphery (dotted lines) with or without ABA. The top row shows PYL1-GFP-Emerin that is localized to the nuclear envelope (NE) and endoplasmic reticulum (ER). The nuclear periphery is outlined by dotted white lines (bottom) except for regions next to tethered genomic loci. Scale bars, 10 μm.

FIG. 12 presents graphs of line scans of the fluorescence intensity of labeled Chr3 loci 5 and labeled PYL1-GFP-Emerin without (top) and with ABA treatment (bottom) along the dotted lines as shown in FIG. 11. Chr3 loci are labeled by CRISPR-Cas9 imaging through the addition of the JF549-halotag dye.

FIG. 13 presents graphs of line scans of the fluorescence intensity of labeled LacO loci without (top) and with ABA treatment (bottom) along the dotted lines as shown.

FIG. 14 is a summary of chromosome locations of less repetitive regions targeted by CRISPR-GO in FIG. 8 and FIG. 9. A single sgRNA binds to multiple repeats (solid grey boxes) within the targeted regions. The genes adjacent to the targeted site are shown in italic letters in grey-outlined boxes.

FIG. 15 presents representative microscopy images comparing the localization of targeted genomic loci (arrows) labeled by 3D-FISH with or without ABA. Nuclei labeled by DAPI are shown. The nuclear periphery is outlined by dotted white lines except for regions next to tethered genomic loci. Insets show enlarged images of periphery-tethered genomic loci. See FIG. 11 for individual channels. Scale bars, 10 μm.

FIG. 16 presents graphs of quantification of percentages of nuclear periphery localized genomic loci (Chr7, ChrX, and CXCR4) in CRISPR-GO cells transfected with a non-targeting sgRNA. From left to right: Graphs 1, 3, and 5: percentages of the nuclear periphery localized genomic loci; Graphs 2, 4, and 6: percentages of cells containing at least one periphery-associated locus.

FIG. 17 presents a summary of chromosome locations of non-repetitive regions targeted by CRISPR-GO in FIG. 18 and FIG. 19. Multiple sgRNAs are designed to tile along the regions upstream or within the gene bodies of the targeted genes (XIST, PTEN, CXCR4). The sgRNA-targeted regions are shown in solid grey boxes. The top grey boxes show sgRNA targets within the forward strand and bottom grey boxes show sgRNAs targets within the reverse strand. The genes adjacent to the targeted site are shown in in italic letters in grey boxes.

FIG. 18 presents graphs of quantification of CRISPR-GO-induced nuclear repositioning efficiency of non-repetitive endogenous genomic loci. The non-repetitive locus adjacent to CXCR4 was targeted with a single sgRNA or multiple sgRNAs pooled together. Genomic loci were visualized by 3D-FISH and nuclei are stained by DAPI. From left to right: Graphs 1 and 3: the percentage of genomic loci at the nuclear periphery; Graphs 2 and 5: the percentage of cells containing at least one nuclear periphery-associated locus. The numbers of loci and cells analyzed are on the bottom.

FIG. 19 presents graphs of a comparison of re-localization efficacy targeting CXCR4 loci using single sgRNAs (sgCXCR4-1, left; sgCXCR4-2, middle) or 6 sgRNAs (right). From left to right, Graphs 1 and 3 (top) and Graph 1 (bottom): the percentage of genomic loci at the nuclear periphery; Graphs 2 and 4 (top) and Graph 2 (bottom): the percentage of cells containing at least one nuclear periphery-associated locus. The numbers of loci and cells analyzed are on the bottom.

FIG. 20 is a graph of the time course of the inducible and reversible repositioning of endogenous locus Chr3:q29, mediated by addition or removal of ABA. The Y axis shows the percentage of periphery-localized Chr3:q29 loci. The X axis shows the time in hours from ABA addition or removal. Data are represented as mean±SEM.

FIG. 21 is a graph of a comparison of the genomic repositioning efficacy in S-phase arrested cells (+ABA, +HU) and control cells (+ABA, −HU) at different time points after ABA addition. The Y axis shows the percentage of periphery-localized Chr3:q29 loci at different time points. Data are represented as mean±SEM. The box on the left shows the outline of the time-course experiment.

FIG. 22 presents representative microscopy images showing mitosis-independent tethering of endogenous Chr3:q29 loci (arrow) to the nuclear envelope. A Chr3:q29 locus (arrow) starts off separate from the nuclear envelope in the first 4 h of recording. Nuclear periphery tethering occurs at 4.5 h and remains stable for the rest of the 8 h of recording. Images here are insets in FIG. 23. Scale bar, 2 μm.

FIG. 23 presents representative microscopic images showing mitosis-independent tethering of endogenous genomic loci to the nuclear periphery. The insets are also shown in FIG. 22. PYL1-GFP-Emerin is localized to nuclear envelope (NE) and endoplasmic reticulum (ER), and the nuclear envelope is outlined by dotted lines. A Chr3 locus is not adjacent to the nuclear envelope in the first 4 h of recording. Nuclear periphery tethering happens at 4.5 h and remains for the rest of the 8 h of recording. Nuclear rotation happens between 10 h and 12 h. Scale bar, 10 μm.

FIG. 24 is a graph showing the distances between the genomic locus in FIG. 22 and nearest nuclear periphery at different time points. Images were taken every 30 mins.

FIG. 25 presents scatter plots of step displacement (dx, dy) of untethered (1&2) and tethered (3&4) Chr3 loci. The step displacement is calculated by subtracting the position of a previous time point from the new position: dx^{t}=(x^{t}−x^{t-1}) and dy^{t}=y^{t}−y^{t-1}. Movements were tracked every 4 s for 6 min.

FIG. 26 is a graph of the comparison of average step distance of untethered (1696 steps in 19 cells) and tethered (1669 steps in 14 cells) Chr3:q29 loci. p<0.0001 by a two-side t-test with unequal variance. Data are represented as mean±SD.

FIG. 27 is a graph of the fitting of the step distances of untethered and tethered Chr3:q29 loci using gamma distribution. Fitted parameters: shape parameter k=2.4 for untethered loci and 1.9 for tethered loci; rate parameter β=21.9 for untethered loci and 46.3 for tethered loci.

FIG. 28 is a schematic illustration of an ABA-inducible CRISPR-GO system to target genomic loci to CBs through co-expression of ABI-dCas9 and PYL1-GFP-Coilin in human cells. ABA treatment dimerizes ABI and PYL1 and tethers ABI-dCas9-targeted genomic loci to CBs containing PYL1-GFP-Coilin.

FIG. 29 presents representative microscopic images showing the colocalization of the targeted LacO loci (top panels, by FISH) and Coilin-GFP-labeled CBs (middle panels) with or without ABA.

FIG. 30 presents graphs of quantification of CRISPR-GO-induced CB tethering efficiency of LacO loci. From left to right, Graph 1: the percentage of LacO loci that co-localize with Coilin-GFP labeled CBs; Graph 2: the percentage of cells containing at least one CB-colocalized LacO locus. The number of loci and cells analyzed are labeled on the bottom. Data are represented as mean±SEM.

FIG. 31 presents representative microscope images showing the colocalization of other CB components (SMN, Fibrillarin, Gemin2, by immunostaining) with LacO loci (by FISH) using the CRISPR-GO system to tether LacO loci to CBs.

FIG. 32 presents representative microscopic images showing colocalization of targeted Chr3:q29 loci (top panels, by CRISPR-Cas9 imaging) and Coilin-GFP labeled CBs (middle panels) with or without ABA.

FIG. 33 presents graphs of quantification of CRISPR-GO induced CB-tethering efficiency of Chr3:q29 loci. From left to right, Graph 1: the percentage of Chr3:q29 loci that co-localize with CBs; Graph 2: the percentage of cells containing at least one CB-colocalized Chr3:q29 locus. The numbers of loci and cells are on the bottom. Data are represented as mean±SEM.

FIG. 34 is a schematic illustration of an ABA-inducible CRISPR-GO system to target genomic loci to PML bodies through co-expression of ABI-dCas9 and PYL1-GFP-PML.

FIG. 35 presents representative microscopic images showing colocalization of targeted Chr3:q29 loci (top panels, by CRISPR-Cas9 imaging) and PML-GFP labeled PML bodies (middle panels) with or without ABA.

FIG. 36 presents graphs of quantification of CRISPR-GO-induced PML body tethering efficiency to the targeted Chr3:q29 loci. From left to right, Graph 1: the percentage of Chr3:q29 loci that colocalize with PML bodies; Graph 2: the percentage of cells containing at least one PML body-colocalized Chr3:q29 locus. The numbers of loci and cells are on the bottom. Data are represented as mean±20 SEM.

FIG. 37 presents representative microscopic images showing colocalization of another PML body marker, SP100 (immunostaining), with Chr3:q29 loci (CRISPR-Cas9 imaging) after using CRISPR-GO to tether Chr3:q29 loci to PML bodies. Scale bars, 10 μm.

FIG. 38 is a graph of rapidly inducible chromatin-CBs association through addition of ABA. The Y axis shows the percentage of CB-colocalized LacO loci. Data are represented as mean±SEM.

FIG. 39 is a plot diagram showing dynamics of chromatin-CBs disassociation after removal of ABA. The Y axis shows the percentage of CB-colocalized LacO loci. X axis shows the time in hours from ABA removal. Data are represented as mean±SEM.

FIG. 40 presents a comparison of GFP-Coilin fluorescence at targeted LacO loci in cells treated with ABA (top) and 6 hours after ABA removal (bottom two rows). Two representative microscopic images are shown for cells with dimmed CBs (middle) or cells in which GFP-Coilin CBs have disappeared (bottom). Line scan (right) measures the raw fluorescence intensity of GFP-Coilin and LacO loci along the dotted lines shown on the left.

FIG. 41 presents representative real-time microscopic images showing the rapid formation of a de novo CB (Coilin) at the targeted LacO locus mediated by CRISPR-GO. The chosen cell was imaged first before ABA treatment (−150 s). ABA was added to the culture medium between −150 s and 0 s, and 0 s represents the first image taken of the same cell immediately after ABA addition.

FIG. 42 shows repression of endogenous gene expression adjacent to targeted loci and across long distances by Cajal body colocalization. Left: schematic illustration of the CRISPR-GO system to colocalize the Chr3:q29 locus to CBs in U2OS cells. ACAP2 is located ˜35 kb upstream of the sgRNA target site, and PPP1R2 is located ˜36 kb downstream of the sgRNA target site. Right: Graph of comparison of ACAP2 and PPP1R2 gene expression (measured by RT-qPCR) using CRISPR-GO to colocalize Chr3:q29 loci to CBs in +/− ABA conditions. See FIG. 43 for controls. No ABA is represented in dark gray and with ABA is represented in light gray in the graph.

FIG. 43 presents graphs of controls for using CRISPR-GO to colocalize the endogenous Chr3 loci with CBs. Left: measurement of ACAP2 and PPP1R2 mRNA expression with the CRISPR-GO system but without a targeting sgRNA with and without ABA; Right: measurement of ACAP2 and PPP1R2 mRNA expression with ABI-dCas9 and a Chr3-targeting sgRNA, but without PYL1-GFP-Coilin with and without ABA. mRNA was measured using RT-qPCR under different conditions. No ABA is represented in dark gray and with ABA is represented in light gray in the graphs.

FIG. 44 is a graph of quantification of the Coilin-GFP fluorescence intensity at the targeted LacO loci shown in FIG. 41. The fluorescence intensity before ABA addition at −150 s was set to 0 (background).

FIG. 45 presents real-time microscopic images showing colocalization of an existing CB (Coilin, arrow) to an adjacent targeted LacO locus mediated by CRISPR-GO. The chosen cell was imaged before ABA treatment (−200 s). ABA was added to the culture medium between −200 s and 0 s, and 0 s represents the first image taken immediately after ABA addition. Scale bars, 10 μm.

FIG. 46 shows adjacent reporter gene expression repressed by repositioning targeted chromatin DNA to the nuclear periphery. Left: schematic illustration of the CRISPR-GO system to reposition a LacO repeat array to the nuclear periphery in the U2OS 2-6-3 cells, which is inserted adjacent to a Doxycycline (Dox)-inducible TRE-miniCMV promoter driving a CFP-SKL reporter gene. Right: graph of comparison of CFP reporter expression level using the CRISPR-GO system to reposition LacO loci to the nuclear periphery in +/− Dox and +/− ABA conditions. Data are represented as mean±SD. No ABA is represented in dark gray and with ABA is represented in light gray in the graph. No ABA is represented in dark gray and with ABA is represented in light gray in the graph. See FIG. 47 for representative histograms and controls.

FIG. 47 presents representative flow cytometry histograms comparing the fluorescence intensity of CFP reporter expression using CRISPR-GO tethering of LacO loci to the nuclear periphery under different treatments. The statistics diagram is shown in FIG. 46. The right diagram shows the quantification of relative CFP fluorescence with a non-targeting sgRNA with or without ABA treatment for +/− Dox. No ABA is represented in dark gray and with ABA is represented in light gray in the graph. Data are represented as mean±SDs.

FIG. 48 presents graphs of the comparison of ACAP2 and PPP1R2 gene expression when using the CRISPR-GO system to reposition Chr3 loci to the nuclear periphery. mRNA was measured using RT-qPCR under different conditions. Cells transfected with a non-targeting sgRNA (sgNT) were used as control. Data are represented as mean±SD.

FIG. 49 shows reporter gene expression adjacent to targeted loci repressed by Cajal body colocalization. Left: schematic illustration of the CRISPR-GO system to colocalize the LacO repeat array to CBs in the U2OS 2-6-3 cells. Right: graph of comparison of CFP reporter expression using the CRISPR-GO system to colocalize LacO loci to CBs for +/− Dox and +/−ABA conditions. No ABA is represented in dark gray and with ABA is represented in light gray in the graph. See FIG. 50 for representative histograms and controls.

FIG. 50 presents representative flow cytometry histograms comparing the fluorescence intensity of CFP reporter expression using CRISPR-GO tethering LacO loci to CBs under different treatments. The statistics diagram is shown in FIG. 49. The right diagram shows the quantification of relative CFP fluorescence with a non-targeting sgRNA with or without ABA treatment for +/− Dox. With a non-targeting sgRNA, ABA treatment leads to slight but insignificant decrease (p>0.05) in CFP reporter expression. Data are represented as mean±SDs. No ABA is represented in dark gray and with ABA is represented in light gray in the graph.

FIG. 51 presents histograms of distances between telomeres and the nearest nuclear envelope point during interphase in example cells treated with or without ABA.

FIG. 52 is a graph of the comparison of relative cell viability as measured by an Alamar blue assay after using the CRISPR-GO system to reposition telomeres to the nuclear envelope. Data are represented as mean±SD.

FIG. 53 shows a cell cycle analysis of cells using CRISPR-GO to reposition telomeres to the nuclear periphery. Cells were treated with ABA for 3 days. Top: representative flow cytometry histograms (left) compare the fluorescence Hoechst 33342 stained DNA components in telomere-targeting ABA treated cells (treated) and non-targeting sgRNA control cells. Bottom: quantification of three experimental replicates. Data are represented as mean±SDs.

FIG. 54 presents representative microscopic images of U2OS cells using CRISPR-GO to colocalize telomeres (TRF1-mCherry, top) and CBs (GFP-Coilin, middle) with or without ABA. Scale bars, 10 μm.

FIG. 55 presents representative microscopic images of HeLa cells using CRISPR-GO to colocalize telomeres (TRF1-mCherry, top) and CBs (GFP-Coilin, middle) with or without ABA. Scale bars, 10 μm.

FIG. 56 is a graph of the comparison of relative U2OS cell viability as measured by an Alamar blue assay using the CRISPR-GO system for targeting telomeres to CBs with or without ABA. Cells were treated with ABA for two days. Data are represented as mean±SD.

FIG. 57 is a graph of the comparison of relative cell viability as measured by an Alamar blue assay of U2OS cells with or without ABA. Cells were treated with ABA for two days. Data are represented as mean±SD.

FIG. 58 shows the CRISPR-GO system enabling programmable control of 3D genome organization relative to other nuclear compartments, thus expanding the CRISPR-Cas toolbox for genome engineering. The CRISPR-GO method allows for programmable control of the 3D genomic positioning and organization of targeted chromatin loci relative to diverse nuclear compartments. This expands the utility of the CRISPR-Cas toolbox beyond applications such as gene editing, transcriptional regulation, epigenetic modification.

FIG. 59 is a schematic illustration of an ABA-inducible CRISPR-GO system to target genomic loci to heterochromatin through co-expression of ABI-dCas9 and PYL1-GFP-HP1a in human cells. Also presented are representative microscopic images showing that ABA treatment dimerizes ABI and PYL1 and colocalizes ABI-dCas9-targeted genomic loci to PYL 1-GFP-HP1α. Scale bars, 10 μm.

FIG. 60 is a graph of the distribution of repetitive sequences (four or more) for each human chromosome and their relative coordinates.

FIG. 61 is a graph of a genome-wide bioinformatics analysis revealing the percentage of human genes located within a given distance to adjacent repetitive sequences.

FIG. 62 shows an overview of the CRISPR-GO system 3D genome organization platform.

FIG. 63 shows a schematic of inducible dCas9 bases strategies for generation of large-scale heterodimerization domains. FIG. 63A shows an inducible dCas9 system that recruits heterochromatin proteins such as HP1a to target sites via heterodimerization of the ABI and PYL1 domains upon addition of the plant hormone abscisic acid (ABA). FIG. 63B shows high local concentrations of heterochromatin proteins generated by targeting tandem repeat regions. A single sgRNA sequence allows potential binding of hundreds of genomic sites in close proximity to one another due to the region comprising tandem repeats. Furthermore, HP1α spontaneously forms homodimers via its chromoshadow domain and has been reported to oligomerize via N-terminus to hinge region interactions. These mechanisms enable a single HP1α to recruit additional HP1α to the target site. FIG. 63C shows that engineered protein scaffolds can further increase the number of copies of heterochromatin proteins bound to each dCas9.

FIG. 64 shows representative microscopic images showing colocalization (arrows) of targeted Chr3:q29 loci (left panel by CRISPR-Cas9 imaging), ABI-BFP-dCas9 (middle panel), PYL1-sfGFP-HP1α labeled (third panel) and a composite image (right panel) with or without ABA.

FIG. 65 shows representative real-time microscopic images (0-60 minutes) showing the rapid formation of de novo heterochromatin loci PYL1-sfGFP-HP1α after ABA addition. The chosen cell was imaged first before ABA treatment (0 min). ABA was added to the culture medium between 0 min and 1 min, and 1 min represents the first image taken of the same cell immediately after ABA addition.

FIG. 66 shows repression of endogenous gene expression adjacent to targeted loci and across long distances by recruitment of PYL1-sfGFP-HP1α to Chr3q29 locus. FIG. 66A shows the schematic illustration of the CRISPR-GO system by recruitment of PYL1-sfGFP-HP1α to the Chr3:q29 locus in U2OS cells. ACAP2 is located ˜35 kb upstream of the sgRNA target site, and PPP1R2 is located ˜36 kb downstream of the sgRNA target site. TFRC is located about 575 kb downstream of the sgRNA target site. FIG. 66B shows the graph of comparison of ACAP2, PPP1R2, and TFRC gene expression (measured by RT-qPCR) using CRISPR-GO to colocalize PYL1-sfGFP-HP1α to the Chr3:q29 loci in +/− ABA conditions (− ABA: DMSO). Data are represented as mean±SD.

FIG. 67 show that synthetic HP1α foci are competent for recruiting free HP1a. Free-floating mCherry-HP1α (right panel) was recruited to the synthetic heterochromatin foci (arrows) formed by PYL1-sfGFP-HP1α (middle panel) and ABI-BFP-dCas9 (left panel), after 2 days and 6 days of ABA treatment.

FIG. 68 shows co-localization of mCherry-HP1α with synthetic HP1α foci. FIG. 68A and FIG. 68B shows fluorescence images of composite images (left panel) showing co-localization of mCherry-HP1α loci and synthetic HP1α foci (indicated by arrows). The right panel shows the linear trace of two foci for each image with a line. Normalized linear fluorescence profile of the co-localization of the mCherry-HP1a with synthetic heterochromatin puncta show selective enrichment at peaks of PYL1-sfGFP-HP1α and ABI-BFP-dCas9 (graphs). For both graphs, the top dark line is mCherry-HP1a, the light gray line is PYL1-sfGFP-HP1a, and the bottom gray line is ABI-BFP-dCas9.

FIG. 69 show that HP1f3 colocalization with synthetic HP1α foci can be observed at rare bright puncta. Cells that were immunostained with HP113 (right panel) localized to the synthetic heterochromatin foci formed by PYL1-sfGFP-HP1α (middle panel) and ABI-BFP-dCas9 (left panel) at bright puncta (solid arrow) but not other puncta (hollow arrows), after 2 days ABA treatment.

FIG. 70 shows that local chromatin density does not increase at synthetic HP1α foci. FIG. 70A and FIG. 70B show representative fluorescence images taken 2 days and 5 days after ABA treatment respectively, representing histone density as measured by mCherry-H2B fluorescence signal (third panel) at the synthetic heterochromatin foci (arrows) formed by PYL1-sfGFP-HP1α (second panel) and ABI-BFP-dCas9 (first panel), and corresponding normalized linear fluorescence profile of the of the mCherry-H2B signal at the synthetic heterochromatin puncta showing no selective enrichment of mCherry-H2B at peaks of PYL1-sfGFP-HP1α and ABI-BFP-dCas9 (graphs). For both graphs, the top line is mCherry-H2B, the light gray line is PYL1-sfGFP-HP1a, and the bottom gray line is ABI-BFP-dCas9.

FIG. 71 shows that local chromatin density does not increase at synthetic HP1α foci. FIG. 71A and FIG. 71B shows representative fluorescence images representing two far-red DNA stains SiR-DNA (minor groove intercalator) and DRAQ5 (major groove intercalator) taken at 2 days after ABA treatment respectively. FIG. 71A shows DNA density as measured by SiR-DNA staining (third panel, top), at the synthetic heterochromatin foci (arrows) formed by PYL1-sfGFP-HP1α (second panel, top) and ABI-BFP-dCas9 (first panel, top) and corresponding normalized linear fluorescence profile of SiR-DNA at synthetic heterochromatin puncta showing no selective enrichment of SiR-DNA at peaks of PYL1-sfGFP-HP1α and ABI-BFP-dCas9 (graph, top). For the graph, the line with three peaks is the siR-DNA staining, and lines that are overlapping with two peaks are the PYL1-sfGFP-HP1a (higher first peak) and ABI-BFP-dCas9 (lower first peak). FIG. 71B shows DNA density as measured by DRAQ5 DNA staining (third panel, bottom) at synthetic heterochromatin foci (arrows) formed by PYL1-sfGFP-HP1a (second panel, bottom) and ABI-BFP-dCas9 (first panel, bottom) and corresponding normalized linear fluorescence profile of the DRAQ5 signal at synthetic heterochromatin puncta showing no selective enrichment of DRAQ5 at peaks of PYL1-sfGFP-HP1a and ABI-BFP-dCas9 (graph, bottom). For both graphs, the top line is mCherry-H2B, the light gray line is PYL1-sfGFP-HP1a, and the bottom gray line is ABI-BFP-dCas9. For the graph, the higher line is the DRAQ5 DNA staining, and lines that are overlapping with two peaks are the PYL1-sfGFP-HP1α and ABI-BFP-dCas9.

FIG. 72 shows that H3K9me3 is not enriched at synthetic HP1α foci (arrows). FIG. 72A and FIG. 72B show representative fluorescence images taken 2 days and 5 days after ABA treatment respectively. The synthetic heterochromatin foci formed by PYL1-sfGFP-HP1α (second panel) and ABI-BFP-dCas9 (first panel) are represented by arrow, and immunostaining with H3K9me3 is shown in the third panel. Corresponding normalized linear fluorescence profile of the co-localization of the H3K9me3 with synthetic heterochromatin puncta showing no selective enrichment of H3K9me3 at peaks of PYL1-sfGFP-HP1α and ABI-BFP-dCas9 is shown by the graphs. For the both graphs, the line with the first peak is the H3K9me3 staining, and lines that are overlapping with two peaks are the PYL1-sfGFP-HP1α and ABI-BFP-dCas9.

FIG. 73 shows that the co-repressor protein KAP1 is not enriched at synthetic HP1α foci. Representative fluorescence images taken 2 days after ABA treatment, depicting cells immunostained with KAP1 (third panel) and synthetic heterochromatin foci (arrows) formed by PYL1-sfGFP-HP1α (second panel) and ABI-BFP-dCas9 (first panel) are shown. Corresponding normalized linear fluorescence profile of the co-localization of the KAP1 with synthetic heterochromatin puncta showing no selective enrichment of KAP1 at peaks of PYL1-sfGFP-HP1α and ABI-BFP-dCas9 is shown in the graph. For the graph, the dark higher line is the KAP1 staining, and lines that are overlapping with two peaks are the PYL1-sfGFP-HP1α and ABI-BFP-dCas9.

FIG. 74 shows that wild-type HP1α caused repression of endogenous gene expression adjacent to and across long distances caused by recruitment of PYL1-sfGFP-HP1α to targeted Chr3q29 loci. FIG. 74A shows the schematic illustration of the CRISPR-GO system by recruitment of PYL1-sfGFP-HP1α to sgRNA target sites at the Chr3:q29 locus in U2OS cells. ACAP2 is located ˜35 kb upstream of the sgRNA target site, PPP1R2 is located ˜36 kb downstream of the sgRNA target site, and TFRC is located about 575 kb downstream of the sgRNA target site. FIG. 74B shows the graph of comparison of PPP1R2 gene expression (measured by RT-qPCR) in U2OS cells expressing wild-type HP1α (HP1α-WT), mutant HP1α (CSD), or mutant HP1α (165E) with PYL1-sfGFP-HP1α localized to the Chr3:q29 loci in +/−ABA conditions (−ABA: DMSO, light gray (left bars for each treatment); +ABA: 100 uM ABA; dark gray (right bars for each treatment). Y axis: Relative fold change; X-axis from left to right: HP1α(WT); HP1α(CSD); HP1α(I165E). FIG. 74C shows the graph of comparison of ACAP2 gene expression (measured by RT-qPCR) in U2OS cells expressing wild-type HP1α, mutant HP1α (CSD), or mutant HP1α (165E) with PYL1-sfGFP-HP1α localized to the Chr3:q29 loci in +/− ABA conditions (−ABA: DMSO, light gray (left bars for each treatment); +ABA: 100 uM ABA; dark gray (right bars for each treatment). Y axis: Relative fold change; X-axis from left to right: HP1α(WT); HP1α(CSD); HP1α(I165E). FIG. 74D shows the graph of comparison of TFRC gene expression (measured by RT-qPCR) in U2OS cells expressing wild-type HP1α, mutant HP1α (CSD), or mutant HP1α (165E) with PYL1-sfGFP-HP1α localized to the Chr3:q29 loci in +/− ABA conditions (−ABA: DMSO, light gray (left bars for each treatment); +ABA: 100 uM ABA; dark gray (right bars for each treatment). Y axis: Relative fold change; X-axis from left to right: HP1α(WT); HP1α(CSD); HP1α(I165E). Data are represented as mean±SD.

FIG. 75 shows that loss of dimerization, but not the chromodomain, abolishes free HP1α at synthetic HP1α foci (arrows). FIG. 75A shows representative fluorescence images taken 2 days after ABA treatment, showing recruitment of free-floating mCherry-HP1α (third panel) to the synthetic heterochromatin foci (arrows) formed by PYL1-sfGFP-HP1α (CSD) (second panel) and ABI-BFP-dCas9 (first panel), and a corresponding normalized linear fluorescence profile of the co-localization of the mCherry-HP1α with synthetic heterochromatin puncta as seen by selective enrichment of mCherry-HP1α at peaks of PYL1-sfGFP-HP1α and ABI-BFP-dCas9 (graph). For the graph, the dark highest line is for mCherry-HP1α, the middle light line is for PYL1-sfGFP-HP1α (CSD), and the lower dark line is for ABI-BFP-dCas9. FIG. 75B shows representative fluorescence images taken 2 days after ABA treatment, representing no recruitment of free-floating mCherry-HP1α (third panel) to the synthetic heterochromatin foci (arrows) formed by PYL1-sfGFP-HP1α (I165E) (second panel) and ABI-BFP-dCas9 (first panel) and corresponding normalized linear fluorescence profile of the co-localization of the mCherry-HP1α with synthetic heterochromatin puncta showing no selective enrichment of mCherry-HP1α at peaks of PYL1-sfGFP-HP1α and ABI-BFP-dCas9 (graph). For the graph, the dark highest line is for mCherry-HP1α, the middle light line is for PYL1-sfGFP-HP1α (I165E), and the lower dark line is for ABI-BFP-dCas9.

FIG. 76 shows that loss of chromodomain impairs normal HP1α nuclear distribution. FIG. 76A shows representative fluorescence images taken 2 days after ABA treatment, representing normal HP1α nuclear distribution as seen by mCherry-HP1α fluorescence (middle panel) impaired HP1α nuclear distribution as seen by PYL1-sfGFP-HP1α (CSD) (left panel), and the corresponding normalized linear fluorescence profile of the mCherry-HP1α and PYL1-sfGFP-HP1α (CSD) signals (graph) showing reduced dynamic range for PYL1-sfGFP-HP1α (CSD). For the graph, the light highest line is for PYL1-sfGFP-HP1α (CSD) and lower line is for mCherry-HP1α. FIG. 76B shows representative fluorescence images taken 2 days after ABA treatment, representing normal HP1α nuclear distribution as seen by mCherry-HP1α fluorescence (middle panel), normal HP1α nuclear distribution as seen by PYL1-sfGFP-HP1α (WT) fluorescence (left panel), and the corresponding normalized linear fluorescence profile of the mCherry-HP1α and PYL1-sfGFP-HP1α (WT) signals (graph) showing equivalent dynamic range for PYL1-sfGFP-HP1α (WT). For the graph, the lines are approximately overlapping and are for PYL1-sfGFP-HP1α (WT) (lighter line) and mCherry-HP1α (darker line).

FIG. 77 shows that HP1α localization is weakly enriched at KRAB-tethered foci. FIG. 77A shows representative fluorescence images taken 2 days after ABA treatment, showing recruitment of free-floating mCherry-HP1α (third panel, top) to the synthetic heterochromatin foci (arrows) shown by PYL1-sfGFP-KRAB (second panel, top) and ABI-BFP-dCas9 (first panel, top), and the corresponding normalized linear fluorescence profile of the co-localization of the mCherry-HP1α with synthetic heterochromatin puncta as shown by the selective enrichment of mCherry-HP1α at peaks of PYL1-sfGFP-KRAB and ABI-BFP-dCas9 (graph, top). For the graph, the highest dark line is for mCherry-HP1α, the light middle line is for PYL1-sfGFP-KRAB, and lowest dark line is for ABI-BFP-dCas9. FIG. 77B shows representative fluorescence images taken 2 days after ABA treatment, showing recruitment of free-floating mCherry-HP1α (third panel, bottom) to the synthetic heterochromatin foci (arrows) shown by PYL1-sfGFP-KRAB (second panel, bottom) and ABI-BFP-dCas9 (first panel, bottom), and the corresponding normalized linear fluorescence profile of the co-localization of the mCherry-HP1α with synthetic heterochromatin puncta as shown by selective enrichment of mCherry-HP1α at peaks of PYL1-sfGFP-KRAB and ABI-BFP-dCas9 (graph). For the graph, the highest dark line is for mCherry-HP1α, the light middle line is for PYL1-sfGFP-KRAB, and lowest dark line is for ABI-BFP-dCas9.

FIG. 78 shows that KRAB recruits KAP1 to synthetic foci. FIG. 78A shows representative fluorescence images taken 2 days after ABA treatment, representing cells immunostained for KAP1 (third panel, top) and synthetic heterochromatin foci (arrows) shown by PYL1-sfGFP-KRAB (middle panel, top) and ABI-BFP-dCas9 (left panel, top), and the corresponding normalized linear fluorescence profile of the co-localization of KAP1 with synthetic heterochromatin puncta as shown by selective enrichment of KAP1 at peaks of PYL1-sfGFP-KRAB and ABI-BFP-dCas9 (top graph). For the graph, the highest gray line is for KAP1 and the other two approximately overlapping lines are for PYL1-sfGFP-KRAB (lighter line) and for ABI-BFP-dCas9 (darker line). FIG. 78B shows representative fluorescence images taken 2 days after ABA treatment, showing cells immunostained with KAP1 (third panel, bottom) and the synthetic heterochromatin foci (arrows) shown by PYL1-sfGFP-KRAB (second panel, bottom) and ABI-BFP-dCas9 (first panel, bottom), and the corresponding normalized linear fluorescence profile of the co-localization of the KAP1 with synthetic heterochromatin puncta as shown by selective enrichment of KAP1 at peaks of PYL1-sfGFP-KRAB and ABI-BFP-dCas9 (graph, bottom). For the graph, the highest gray line is for KAP1 and the other two approximately overlapping lines are for PYL1-sfGFP-KRAB (lighter line) and for ABI-BFP-dCas9 (darker line).

FIG. 79 shows that KRAB-based foci remain deficient for H3K9me3 enrichment. FIG. 79A shows representative fluorescence images taken 2 days after ABA treatment, showing cells immunostained for H3K9me3 (third panel, top), synthetic heterochromatin foci (arrows) shown by PYL1-sfGFP-KRAB (second panel, top) and ABI-BFP-dCas9 (right panel, top), and the corresponding normalized linear fluorescence profile of the co-localization of H3K9me3 with synthetic heterochromatin puncta as shown by no selective enrichment of H3K9me3 at peaks of PYL1-sfGFP-KRAB and ABI-BFP-dCas9 (graph, top). For the graph, the medium gray line is for H3K9me3, the light gray line is for PYL1-sfGFP-KRAB, and the dark gray line is for ABI-BFP-dCas9. FIG. 79B shows representative fluorescence images taken 2 days after ABA treatment, representing cells immunostained with H3K9me3 (third panel, bottom) and synthetic heterochromatin foci (arrows) shown by PYL1-sfGFP-KRAB (second panel, bottom) and ABI-BFP-dCas9 (left panel, bottom), and the corresponding normalized linear fluorescence profile of the co-localization of the H3K9me3 with synthetic heterochromatin puncta showing no selective enrichment of H3K9me3 at peaks of PYL1-sfGFP-KRAB and ABI-BFP-dCas9 (graph, bottom). For the graph, the medium gray line is for H3K9me3, the light gray line is for PYL1-sfGFP-KRAB, and the dark gray line is for ABI-BFP-dCas9.

FIG. 80 shows that chromatin and DNA density does not increase within KRAB-tethered foci. FIG. 80A shows representative fluorescence images taken 2 days after ABA treatment, showing histone density as measured by mCherry-H2B (third panel, top) fluorescence signal at the synthetic heterochromatin foci (arrows) shown by PYL1-sfGFP-KRAB (second panel, top) and ABI-BFP-dCas9 (first panel, top) and the corresponding normalized linear fluorescence profile of the mCherry-H2B signal at synthetic heterochromatin puncta showing no selective enrichment of mCherry-H2B at peaks of PYL1-sfGFP-KRAB and ABI-BFP-dCas9 (graph, top). For the graph, the highest dark line is for H2B-mCherry, the light gray line is for PYL1-sfGFP-KRAB, and the lowest dark gray line is for ABI-BFP-dCas9. FIG. 80B shows representative fluorescence images taken 2 days after ABA treatment, representing DNA density as measured by SiR-DNA staining (third panel, bottom) at the synthetic heterochromatin foci (arrows) as shown by PYL1-sfGFP-KRAB (second panel, bottom) and ABI-BFP-dCas9 (left panel, bottom), and the corresponding normalized linear fluorescence profile of the siR-DNA signal at synthetic heterochromatin puncta showing no selective enrichment of siR-DNA at peaks of PYL1-sfGFP-KRAB and ABI-BFP-dCas9 (graph, bottom). For the graph, the highest dark line is for siR-DNA, the light gray line is for PYL1-sfGFP-KRAB, and the lowest dark gray line is for ABI-BFP-dCas9.

FIG. 81 show that KRAB fails to recapitulate HP1α repression at Chr3q29. FIG. 81A shows the schematic illustration of the CRISPR-GO system by recruitment of PYL1-sfGFP-HP1α or PYL1-sfGFP-KRAB to sgRNA target sites at the Chr3:q29 locus in U2OS cells. ACAP2 is located ˜35 kb upstream of the sgRNA target site, PPP1R2 is located ˜36 kb downstream of the sgRNA target site and TFRC is located about 575 kb downstream of the sgRNA target site. FIG. 81B shows the graph of comparison of PPP1R2 gene expression (measured by RT-qPCR) in U2OS cells expressing PYL1-sfGFP-HP1α or PYL1-sfGFP-KRAB to the Chr3:q29 loci in +/− ABA conditions (−ABA: DMSO, light gray; +ABA: 100 uM ABA; dark gray). Y axis: Relative fold change; X-axis from left to right: HP1α sgNT; HP1α sgChr3; KRAB sgChr3. FIG. 81C shows the graph of comparison of ACAP2 gene expression (measured by RT-qPCR) in U2OS cells expressing PYL1-sfGFP-HP1α or PYL1-sfGFP-KRAB to the Chr3:q29 loci in +/− ABA conditions (−ABA: DMSO, light gray; +ABA: 100 uM ABA; dark gray). Y axis: Relative fold change; X-axis from left to right: HP1α sgNT; HP1α sgChr3; KRAB sgChr3. FIG. 81D shows the graph of comparison of TFRC gene expression (measured by RT-qPCR) in U2OS cells expressing PYL1-sfGFP-HP1α or PYL1-sfGFP-KRAB to the Chr3:q29 loci in +/− ABA conditions (−ABA: DMSO, light gray; +ABA: 100 uM ABA; dark gray). Y axis: Relative fold change; X-axis from left to right: HP1α sgNT; HP1α sgChr3; KRAB sgChr3. Cells transfected with a non-targeting sgRNA (sgNT) were used as control. Data are represented as mean±SD.

FIG. 82 shows that KRAB but not HP1α represses proximally at Chr1p36 repeat. FIG. 82A shows the schematic illustration of the CRISPR-GO system by recruitment of PYL1-sfGFP-HP1α or PYL1-sfGFP-KRAB to sgRNA target sites at the Chr1:p36 locus in U2OS cells. CPTP is located ˜25 kb upstream of the sgRNA target site, INTS11 is located ˜26 kb upstream of the sgRNA target site, and DVL1 is located about 0.9 kb upstream of the sgRNA target site.

FIG. 82B shows the graph of comparison of DVL1 gene expression (measured by RT-qPCR) in U2OS cells expressing PYL1-sfGFP-HP1α or PYL1-sfGFP-KRAB to the Chr1:p36 loci in +/−ABA conditions (−ABA: DMSO, light gray; +ABA: 100 uM ABA; dark gray). Y axis: Relative fold change; X-axis from left to right: HP1α sgNT; HP1α sgChr1; KRAB sgChr1. FIG. 82C shows the graph of comparison of CPTP gene expression (measured by RT-qPCR) in U2OS cells expressing PYL1-sfGFP-HP1α or PYL1-sfGFP-KRAB to the Chr1:p36 loci in +/− ABA conditions (−ABA: DMSO, light gray; +ABA: 100 uM ABA; dark gray). Y axis: Relative fold change; X-axis from left to right: HP1α sgNT; HP1α sgChr1; KRAB sgChr1. FIG. 82D shows the graph of comparison of INTS11 gene expression (measured by RT-qPCR) in U2OS cells expressing PYL1-sfGFP-HP1α or PYL1-sfGFP-KRAB to the Chr1:p36 loci in +/− ABA conditions (−ABA: DMSO, light gray; +ABA: 100 uM ABA; dark gray). Y axis: Relative fold change; X-axis from left to right: HP1α sgNT; HP1α sgChr1; KRAB sgChr1. Cells transfected with a non-targeting sgRNA (sgNT) were used as control. Data are represented as mean±SD.

FIG. 83 shows that HP1α and KRAB act antagonistically on gene repression at Chr1p36. FIG. 83A shows the schematic illustration of the recruitment of PYL1-sfGFP-HP1α, PYL1-sfGFP-KRAB or both to sgRNA target sites at the Chr1:p36 locus in U2OS cells. CPTP is located ˜25 kb upstream of the sgRNA target site, INTS11 is located ˜26 kb upstream of the sgRNA target site and DVL1 is located about 0.9 kb upstream of the sgRNA target site. FIG. 83B shows the graph of comparison of DVL1 gene expression (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. FIG. 83C shows the graph of comparison of CPTP gene expression (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. FIG. 83D shows the graph of comparison of gene expression of INTS11 (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. Data are represented as mean±SD.

FIG. 84 shows that HP1α and KRAB act antagonistically on gene repression at Chr3q29. FIG. 84A shows the schematic illustration of the CRISPR-GO system by recruitment of PYL1-sfGFP-HP1α, PYL1-sfGFP-KRAB or both to sgRNA target sites at the Chr3:q29 locus in U2OS cells. ACAP2 is located ˜35 kb upstream of the sgRNA target site, PPP1R2 is located ˜36 kb downstream of the sgRNA target site and TFRC is located about 575 kb downstream of the sgRNA target site. FIG. 84B shows the graph of comparison of PPP1R2 gene expression (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. FIG. 84C shows the graph of comparison of ACAP2 gene expression (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. FIG. 84D shows the graph of comparison of gene expression of TFRC (measured by RT-qPCR) in U2OS cells expressing PYL1-sfGFP-HP1α, PYL1-sfGFP-KRAB or both to the Chr1:p36 locus in +/− ABA conditions (−ABA: DMSO, light gray (left bars for each treatment); +ABA: ABA; dark gray (right bars for each treatment). Y axis: Relative fold change; X-axis from left to right: HP1α; KRAB; HP1α+KRAB. Data are represented as mean±SD.

FIG. 85 shows that H3K9me3 is deposited at a subset of foci by SUV39H1 (full length). FIG. 85 shows representative fluorescence images taken 2 days after ABA treatment, depicting cells immunostained with H3K9me3 (third panel, top and bottom) localized to the synthetic heterochromatin foci formed by PYL1-mCherry-SUV39H1 (second panel, top and bottom) and ABI-BFP-dCas9 (first panel, top and bottom). Corresponding normalized linear fluorescence profiles show the co-localization of the H3K9me3 with synthetic heterochromatin puncta as shown by selective enrichment of H3K9me3 at peaks of PYL1-sfGFP-KRAB and ABI-BFP-dCas9 (graphs, top and bottom). For the graphs, the highest dark line is for H3K9me3, the light gray approximately overlapping lines are for PYL1-mCherry-SUV39H1 and for ABI-BFP-dCas9.

FIG. 86 shows that H3K9me3 is not deposited at foci by SUV39H1(Δ1-76). FIG. 86 shows representative fluorescence images taken 2 days after ABA treatment, depicting cells immunostained with H3K9me3 (right panel, top and bottom) and synthetic heterochromatin foci formed by PYL1-mCherry-SUV39H1 (Δ1-76) (middle panel, top and bottom) and ABI-BFP-dCas9 (left panel, top and bottom).

FIG. 87 shows that H3K9me3 is not deposited at G9a (full length). FIG. 87 shows representative fluorescence images taken 2 days after ABA treatment, depicting cells immunostained with H3K9me3 (right panel, top and bottom) and synthetic heterochromatin foci formed by PYL1-mCherry-G9a (full length) (middle panel, top and bottom) and ABI-BFP-dCas9 (left panel, top and bottom).

FIG. 88 shows that G9a (catalytic domain; G9aΔ1-829) localizes to the cytoplasm. FIG. 88 shows representative fluorescence images taken 2 days after ABA treatment, depicting cells immunostained with H3K9me3 (right panel) localized to the synthetic heterochromatin foci formed by PYL1-mCherry-G9aΔ1-829 (left panel).

FIG. 89 shows that SUV39H1 (full length) does not visibly enrich for HP1α. FIG. 89 shows representative fluorescence images taken 2 days after ABA treatment, depicting cells immunostained with HP1α (third panel, top and bottom) and synthetic heterochromatin foci formed by PYL1-mCherry-SUV39H1 (second panel, top and bottom) and ABI-BFP-dCas9 (first panel, top and bottom), and corresponding normalized linear fluorescence profile of the co-localization of the HP1α with synthetic heterochromatin puncta showing no selective enrichment of HP1α at peaks of PYL1-mCherry-SUV39H1 and ABI-BFP-dCas9 (graphs, top and bottom). For the graphs, the highest dark line is for HP1α, and the approximately overlapping lines are for PYL1-sfGFP-SUV39H1 and for ABI-BFP-dCas9.

FIG. 90 shows that PYL1-sfGFP-HP1α exhibits normal subnuclear localization. FIG. 90A shows the PYL1-sfGFP-HP1α (middle panel, top) nuclear distribution resembles that of immunostained HP1β (right panel, top) but not of ABI-BFP-dCas9 (left panel, top) in the absence of the ABA inducer. FIG. 90B shows the PYL1-sfGFP-HP1α (middle panel, bottom) nuclear distribution resembles that of immunostained H3K9me3 (right panel, bottom) but not of ABI-BFP-dCas9 (left panel, bottom) in the absence of the ABA inducer.

FIG. 91 shows no repression of endogenous gene expression adjacent to targeted loci and across long distances by recruitment of a non-targeting sgRNA to Chr3q29 locus. FIG. 91A shows the schematic illustration of the genomic locations of the measured genes at Chr3:q29 locus in U2OS cells. ACAP2 is located ˜35 kb upstream of the sgRNA target site, and PPP1R2 is located ˜36 kb downstream of the sgRNA target site. TFRC is located about 575 kb downstream of the sgRNA target site. FIG. 91B shows the graph of comparison of ACAP2, PPP1R2, and TFRC gene expression (measured by RT-qPCR) using a non-targeting sgRNA that does not localize to the Chr3:q29 loci in +/− ABA conditions (−ABA: DMSO, light gray (left bars for each treatment); +ABA: ABA; dark gray (right bars for each treatment). Y axis: Relative fold change; X-axis from left to right: ACAP2; PPP1R2; TFRC. Data are represented as mean±SD.

FIG. 92 shows that Chr3q29 loci are not naturally heterochromatic. FIG. 92 shows lack of H3K9me3 colocalization (middle panel, top) or ABI-BFP-dCas9 localization (sgChr3) (left panel, top and bottom) with mCherry-HP1α (middle panel, bottom) in the presence of DMSO, in the absence of the ABA inducer. Corresponding normalized linear fluorescence profile of the co-localization profile of HP1α with heterochromatin shows lack of selective enrichment of HP1α at peaks of H3K9me3 and ABI-BFP-dCas9 (graphs). For the top graph, the light gray higher line is for H3K9me3 and the dark lower line is for ABI-BFP-dCas9. For the bottom graph, the light gray higher line is for mCherry-HP1α and the dark lower line is for ABI-BFP-dCas9.

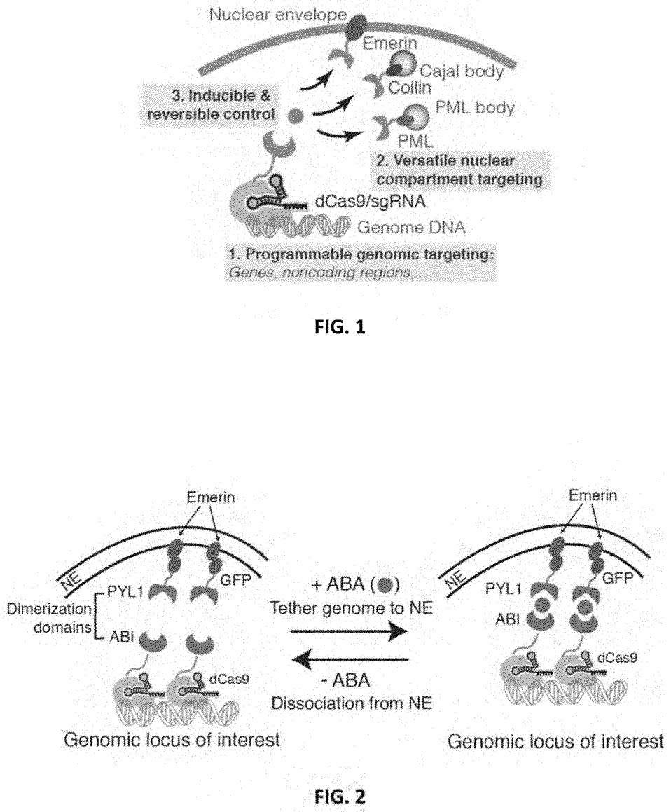

### DETAILED DESCRIPTION OF THE DISCLOSURE

The 3-dimensional (3D) spatial organization of polynucleotides within living cells can play an important role in such processes as regulating and maintaining gene expression, genome stability, and cellular function. For example, genomic sequences that associate with nuclear lamina or the nuclear periphery often exhibit low transcriptional activity, while those that localize to the nuclear interior often exhibit relatively higher activity. Furthermore, the eukaryotic cell nucleus can contain many membraneless nuclear bodies, such as Cajal bodies, PML bodies, nucleolus and speckles, that can be functionally important in a variety of biological processes. A central goal in genomics and cell biology has been to understand the relationship between genome structure, its organization within various nuclear compartments, and gene expression, but this goal has been constrained by currently available methods.

A correlation between genome organization and cell fate determination has been suggested by numerous studies using microscopy-based imaging (e.g., FISH) and chromosome conformation capture (3C) techniques. For example, during lymphocyte development, the IgH and Igx loci that are positioned at the nuclear periphery in progenitor cells often relocate to nuclear interior in pro-B cells, a process that is synchronous with the activation and rearrangement of immunoglobulin loci. Similarly, the genomic locus of the proneural transcription factor Ascl1 can be located in the nuclear periphery in undifferentiated embryonic stem cells, but can relocate to the nuclear interior during neuronal differentiation. Moreover, 3C-based studies have revealed changes in high-resolution chromatin interactions (e.g., topologically associated domains) during development and disease processes. Altogether, these are powerful methods for mapping genome organization and measuring physical interactions of chromatin elements, they often cannot provide causal links between genome positioning and function and they are unable to measure dynamic changes in living cells.

Nuclear compartments have been observed to play an important role in genome organization and function. Nuclear bodies are proposed to assemble through liquid-liquid phase separation, which can be driven by multivalent interactions between proteins and RNAs. De novo nuclear body formation can be nucleated by immobilization of protein or RNA components on chromatin. Among nuclear bodies, Cajal bodies (CBs) can be essential for vertebrate embryogenesis, and can be abundant in tumor cells and neurons. CBs can be marked by a scaffold protein component, Coilin, and can play an important role in small nuclear RNA (snRNA) biogenesis, ribonucleoprotein (RNP) assembly, and telomerase biogenesis. The promyelocytic leukemia (PML) nuclear bodies, marked by a tumor suppressor protein, PML, can be abundant nucleus dot structures that associate with disease processes including tumor and viral infection. However, how the colocalization of nuclear bodies and chromatin can causally affect gene expression, and cellular function remains mostly elusive.

To understand such causal relationships, sequence-specific DNA-protein interactions have been exploited to mediate targeted genomic reorganization. This technique utilizes an array of LacO repeats inserted into a genomic locus, which facilitates tethering of the adjacent genomic sequence to the nuclear periphery when combined with Lad fused to a nuclear membrane protein. Using this technique, several studies have reported that repositioning a gene to the nuclear periphery can lead to gene repression. However, this technique may not suitable for programmable genome targeting, and can be tedious and difficult to implement. For example, creating a stable LacO repeat-containing cell line is a prerequisite for this technique, which already involves many steps such as the random insertion of a large LacO repeat array into the genome, screening for cells containing a single insertion locus, generating stable cell lines, and characterization of the genomic insertion site by FISH. New tools are needed to manipulate the spatial and temporal organization of the genome in a programmable, precise, and targeted manner.

Prokaryotic Class II CRISPR-Cas (Clustered regularly interspaced short palindromic repeats-CRISPR associated) systems can be repurposed as a toolbox (e.g. Cas9 and Cpfl) for gene editing, gene regulation, epigenome editing, chromatin looping, and live-cell genome imaging. Nuclease-deactivated Cas (dCas) proteins coupled with transcriptional effectors or epigenetic modifying domains can allow for regulation of expression of genes adjacent to the single guide RNA (sgRNA) target site. It remains unknown whether the CRISPR-Cas system can be used to mediate genome organization and reposition the location of chromatin DNA relative to various nuclear compartments within mammalian nuclei. In view of the foregoing, there exists a need for alternative systems and methods to carry out the spatial organization of target polynucleotides. The present disclosure addresses this and other needs.

Furthermore, eukaryotic cells are complex structures capable of coordinating numerous biochemical reactions in space and time. Keys to such coordination are both the 3D organization of polynucleotides such as the genome, and the subdivision of intracellular space into functional compartments. Compartmentalization can be achieved by intracellular membranes, which surround organelles and act as physical barriers. In addition, cells have developed sophisticated mechanisms to partition their inner substance in a tightly regulated manner. Recent studies provide compelling evidence that membraneless compartmentalization can be achieved by liquid demixing, a process culminating in liquid-liquid phase separation and the formation of phase boundaries.

The inventors have surprisingly discovered versatile systems and methods that can efficiently control the spatial positioning of polynucleotides relative to the functional compartments, including nuclear compartments such as the nuclear periphery, Cajal bodies, and promyelocytic leukemia (PML) bodies. Additionally, the inventors have discovered versatile systems and methods that can efficiently control the spatial positioning of compartments relative to the polynucleotides. The systems, compositions, and methods can also be useful in generating synthetic phase separations, by forming supramolecular assemblies of proteins, RNA, and/or DNA molecules organized or portioned within a cell. For example, proteins that contribute to the formation of a specific compartment (e.g., a compartment-specific protein or a compartment constituent protein) can be used to form a compartment around a target polynucleotide. The systems and methods disclosed herein can be useful for manipulating the spatiotemporal organization of genomic DNA and RNA components in the nucleus/cytoplasm and for regulating diverse cellular functions. The provided systems, compositions, and methods also can be used for programmable control of spatial genome organization, and for applying this organization to affect polynucleotide regulation and cellular function, and to mediate interacting dynamics between targeted polynucleotides and different cellular compartments. The disclosed systems, compositions, and methods can be used, for example, to achieve the dynamic reorganization of subcellular space as a framework to manipulate pathological protein assembly in diseases including cancer and neurodegeneration. The disclosed systems, compositions, and methods can also be used, for example, to achieve the dynamic reorganization of subcellular space and target polynucleotides to facilitate homology directed repair (HDR), non-homologous end joining (NHEJ), or other methods of polynucleotide repair.

Furthermore, the disclosed systems, compositions, and methods can produce three-dimensional structures including three-dimensional loops, for example, three-dimensional loops between enhancers and promoters, chromosome boundaries, a topologically associating domains, or gene clusters for regulating multiple genes and their regulatory elements. The disclosed systems, compositions, and methods can be used for producing genomic insulators that separate a target region comprising the target polynucleotide for chromosome protection and manipulation. The disclosed systems, compositions, and methods can also be used for localizing polynucleotides within particular types of compartment. For example, the disclosed systems, compositions, and methods, are used to trap a region of polynucleotides comprising the target polynucleotide in a spatial region of compartment or within a compartment that comprises uniquely defined biochemical properties that promote or prevent recombination or mutagenesis, promote or inhibit of gene transcription, splicing, or translation, or promote or inhibit polynucleotide transport and movement. Thus, importantly, the systems, compositions, and methods as described herein can be used to manipulate the fate, function, and properties of target polynucleotide and genomic region comprising the target polynucleotide, which also can be used to manipulate the fate, function, and properties of the cell comprising the target polynucleotide.

The disclosed systems, compositions, and methods can be chemically inducible and reversible, enabling interrogation of real-time dynamics of, for example, chromatin interactions with nuclear compartments in living cells. As further examples, inducible repositioning of genomic loci to the nuclear periphery can allow dissection of mitosis-dependent and -independent relocalization events, interrogation of the relationship between gene position and expression, and understanding of the effects of telomere repositioning on cell growth. The systems, compositions, and methods described herein can mediate rapid de novo formation of Cajal bodies at target chromatin loci and causes significant repression of adjacent endogenous gene expression across long distances (>30 kb). The systems, compositions, and methods described herein can mediate rapid de novo formation of a compartment comprising proteins that facilitate HDR, such as Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1, at a target polynucleotide that has been cut. The systems, compositions, and methods described herein rapid de novo formation of a compartment that facilitates nuclear heterochromatin formation, such as HP1α, HP1β, KAP1, KRAB, SUV39H1, or G9a. The compartment can comprise proteins that are specific for that particular compartment (compartment-specific proteins) or proteins that are part of that particular compartment (compartment-constituent protein). The provided system, compositions, and methods thus offers a novel platform to investigate large-scale spatial polynucleotide organization and function in a targeted manner and a novel platform for manipulating the fate, function, and properties of a target polynucleotide, the region comprising the target polynucleotide, and the cell comprising the target polynucleotide.

In some embodiments, the use of different sgRNAs allows the system, composition, and method to be programmed to flexibly target different genomic sequences. Target polynucleotide colocalization with a compartment can be triggered through rapid de novo compartment formation, formation of a compartment around a target polynucleotide, or through repositioning target polynucleotide to an existing compartment. The repositioning of genomic loci to the nuclear periphery can be enabled in both mitosis-dependent and -independent manners. Target DNA co-localization with Cajal bodies can be triggered through rapid de novo Cajal body formation or through repositioning target DNA to existing Cajal bodies. Targeting genomic loci to the nuclear periphery or to Cajal bodies using the provided systems and methods can also repress adjacent reporter gene expression. Importantly, colocalization of genomic loci with Cajal bodies also can repress expression of adjacent endogenous genes (>30 kb). Furthermore, the sequestering of telomeres to the nuclear periphery using aspects of the present disclosure can negatively impact cell growth. Targeting genomic loci to a compartment or formation of the compartment comprising proteins that facilitate HDR, such as Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1, at a target polynucleotide that has been cut can also be used to facilitate HDR of the cut target polynucleotide. Targeting genomic loci to a compartment or formation of the compartment comprising proteins that facilitates nuclear heterochromatin formation, such as HP1α, HP1β, KAP1, KRAB, SUV39H1, or G9a can be used to facilitate transcription regulation of a genomic region comprising the target polynucleotide.

The CRISPR-Cas system has been repurposed as a flexible genome engineering platform, and has been used for applications such as gene editing, transcriptional regulation, epigenetic modifications, DNA looping, and genome imaging. Provided herein are further expansions to the CRISPR-Cas toolbox in the form of a polynucleotide organization system which enables programmable control of targeted polynucleotide positioning within the cellular compartments. In certain aspects, the targeted polynucleotides comprise genomic DNA and the system is referred to as CRISPR-GO (FIG. 58), wherein GO refers to Genome Organization. The systems and methods disclosed herein can efficiently target polynucleotides (e.g., endogenous genomic loci) to various cellular compartments (e.g., the nuclear periphery, Cajal bodies, and PML bodies) or efficiently assemble or form various cellular compartments around a target polynucleotide. The provided systems, compositions, and methods can be inducible and reversible, allowing for the interrogation of, for example, the interaction dynamics between targeted chromatin DNA and nuclear compartments. The provided systems, compositions, and methods can be inducible and reversible, allowing for spatial and temporal control over the formation of a compartment around a target polynucleotide. Using this feature, both mitosis-dependent and -independent repositioning of genomic loci to the nuclear periphery have been achieved, and both de novo formation of Cajal bodies at the target loci and colocalization of existing Cajal bodies with targeted chromatin loci have been demonstrated. Colocalization of the genomic loci with the nuclear periphery or Cajal bodies using the systems and methods disclosed herein has been used to affect adjacent gene expression. Notably, colocalization of an endogenous locus with Cajal bodies using the provided systems and methods can significantly repress nearby gene expression, even though these genes are far away (>30 kb) from the target site. It has also been found that repositioning telomeres to the nuclear periphery with the systems and methods disclosed herein can disrupt telomere dynamics and reduces cell viability. Targeting genomic loci to a compartment or formation of the compartment comprising proteins that facilitate HDR, such as Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1, at a target polynucleotide that has been cut can also be used to facilitate HDR of the cut target polynucleotide. Targeting genomic loci to a compartment or formation of the compartment comprising proteins that facilitates nuclear heterochromatin formation, such as HP1α, HP1β, KAP1, KRAB, SUV39H1, or G9a can be used to facilitate transcription regulation of a genomic region comprising the target polynucleotide. The provided methods offer a platform for the programmable control of polynucleotide (e.g., genomic DNA) interactions with various cellular (e.g., nuclear) compartments, which can facilitate a deeper understanding of the functional role of spatiotemporal polynucleotide organization in regulation, stability, and cellular function. Furthermore, the systems, compositions, and methods as described herein offer a platform programmable control of the environment of regions of polynucleotides, such as particular regions of genomic polynucleotides within a nucleus, by forming a compartment around a target polynucleotide within the region of polynucleotides, which results in spatiotemporal manipulation of the fate, function, and properties of that region of polynucleotides.

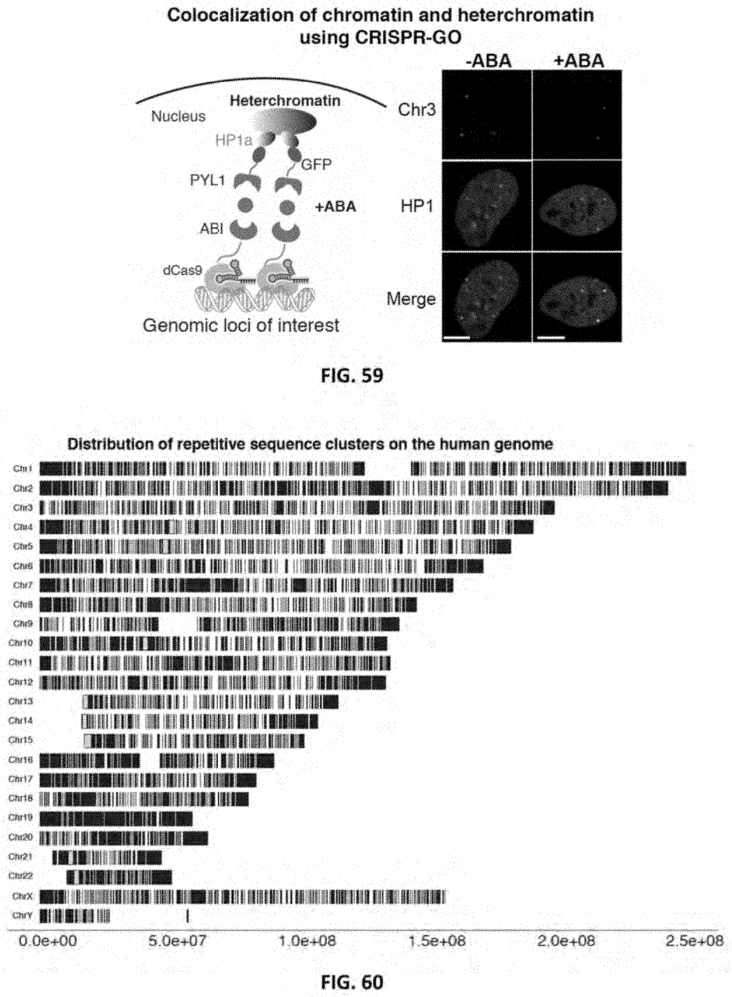

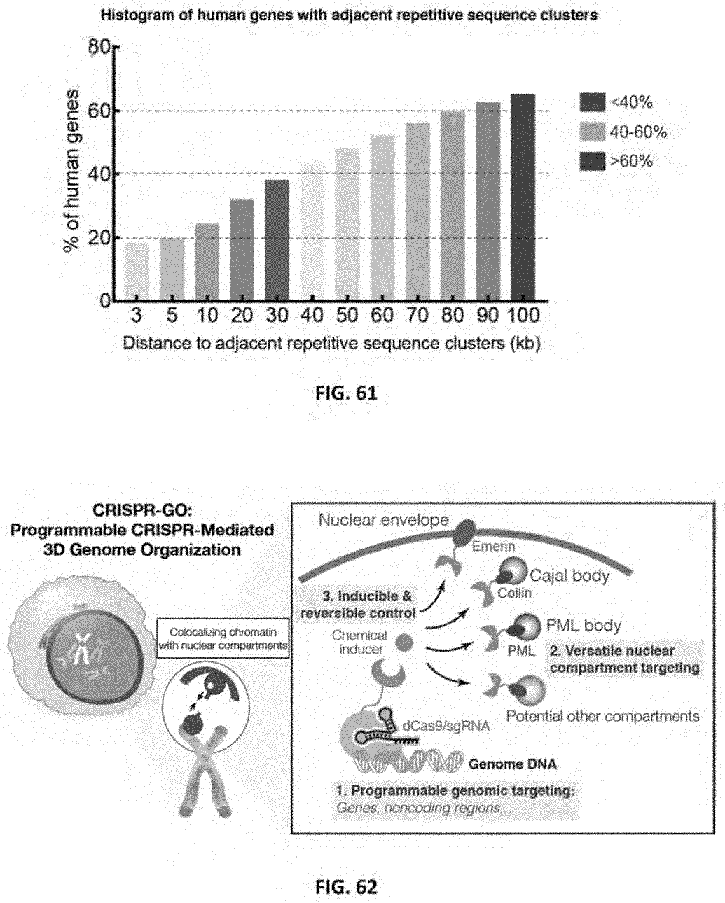

A major goal in cell biology is the understanding of how genomic interactions with different nuclear compartments affect gene expression, chromatin conformation, and cellular functions. The CRISPR-GO system can efficiently target specific genomic loci to the nuclear periphery, Cajal bodies, and PML bodies, and also holds potential to be expanded to other nuclear compartments such as nucleoli, nuclear pore complexes, and nuclear speckles. Targeting genomic loci to other nuclear compartments can be achieved by coupling CRISPR-GO with different compartment-specific proteins, such as heterochromatin protein 1a (HP1α) (FIG. 59). The CRISPR-GO system can also efficiently assemble or form a compartment, such as a compartment comprising Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1, to a target polynucleotide for facilitation of HDR. Assembling or forming other compartments, such as compartment to facilitate NHEJ, can be achieved by coupling CRISPR-GO with different compartment-specific proteins, such as KU70, KU80, Artemis, PKcs, LigIV, or XRCC4. Similarly, the systems and methods disclosed herein provide a versatile modular platform that can be applied to the study of various cellular compartments The CRISPR-GO system can also efficiently assemble or form a compartment, such as a compartment comprising a compartment-constituent proteins, e.g., HP1α, HP1β, KAP1, KRAB, SUV39H1, or G9a, to facilitate nuclear heterochromatin formation around a target polynucleotide. Assembling or forming other compartments around a target polynucleotide can be achieved by coupling CRISPR-GO with different compartment-specific proteins or compartment-constituent proteins that can be used for regulating expression of (e.g., increasing or decreasing), introducing epigenetic modifications to, producing three-dimensional structures, a topologically associating domains, or genomic boundaries comprising the target polynucleotide or an additional polynucleotide (e.g., distal or proximal gene from the target polynucleotide).

The provided systems (e.g., CRISPR-GO), compositions, and methods allows programmable re-localization of polynucleotides (e.g., genomic loci) in a precise and targeted manner. The provided systems (e.g., CRISPR-GO), compositions, and methods allows programmable formation of a compartment using compartment constituent protein around a target polynucleotide (e.g., genomic loci) in a precise and targeted manner. The provided systems (e.g., CRISPR-GO), compositions, and methods also allow for control of the local environment of a target polynucleotide by assembly or co-localization of a compartment to a polynucleotide in a precise and targeted manner. For example, the CRISPR-GO system can efficiently target repetitive and non-repetitive chromatin loci located on different chromosomes to nuclear compartments. As another example, the CRISPR-GO system can efficiently assemble a compartment comprising proteins to facilitate HDR to a target repetitive or non-repetitive chromatin loci located on a chromosome. As an additional example, the CRISPR-GO system efficiently forms a compartment, such as a heterochromatin compartment, around target repetitive and non-repetitive chromatin loci located on different chromosomes. The CRISPR-GO system can efficiently assemble a compartment comprising proteins to facilitate nuclear heterochromatin around a target repetitive or non-repetitive chromatin loci located on a chromosome. Unlike the LacI-LacO system, the genomic targets of the CRISPR-GO system can be flexibly defined by the base-pairing interactions between sgRNAs and the target DNA sequence, and simply altering a ˜20 nt region on the sgRNAs allows for the targeting of a different genomic locus. This programmable feature can allow one to use CRISPR-GO to target a variety of genomic elements, including protein-coding genes, non-coding RNA genes, and regulatory elements. In contrast, the LacO-LacI technique is not suitable for programmable genomic targeting, as it can only be performed on well-characterized cell lines containing a highly repetitive LacO array. Creating and characterizing a useful LacO-containing cell line is difficult and laborious. LacO arrays are usually randomly inserted into the genome, after which cells containing a single-copy insertion are selected to build stable cell lines before the precise genome integration sites is characterized by FISH and other methods. In addition, it is possible that integration of a large LacO array in the genome may alter local chromatin conformation. Altogether, the versatility of the systems, compositions, and methods disclosed herein offers a major technological advantage over conventional methods to study cellular organization and to manipulate the fate, function, and properties of a target polynucleotide, a genomic region comprising the target polynucleotide, or the cell comprising the target polynucleotide.

The overall ease of targeting a new locus of polynucleotides or a compartment with the systems, compositions, and methods disclosed herein can facilitate broader studies of the relationship between perturbations in 3D polynucleotide organization and changes in cellular phenotypes. For example, different sgRNA design strategies can be used to target repetitive and non-repetitive genomic loci. Repetitive genomic loci can be easily targeted using a single sgRNA that has multiple targets within a defined genomic region. The human genome has abundant repetitive or repeat-derived sequences, many of which likely have important genome-organization roles. These repetitive sequences are candidates for large-scale screening experiments, opening the door to more high-throughput approaches to study the relationship between genome organization relative to nuclear compartments and cellular phenotype. In addition, non-repetitive genomic loci can be targeted using multiple sgRNAs or using a single sgRNA. To target a non-repetitive locus, a pool of tiling sgRNAs can be used as a starting point.

The provided systems, compositions, and methods can also be useful for studying real-time dynamics of polynucleotide repositioning and the association and dissociation of cellular compartments from specific regions in living cells. In the CRISPR-GO system, genomic loci are targeted to the desired compartments via chemically induced physical interactions between dCas9-bound genomic loci and compartment-specific proteins. Alternatively, a compartment is targeted or assembled at a target polynucleotide via chemically induced physical interactions between dCas9-bound genomic loci and compartment-specific proteins or compartment-constituent proteins. The inducible and reversible feature of CRISPR-GO prevents potential adverse effects from continuously repositioning chromatin DNA to a given nuclear compartment or continuously repositioning a compartment to a target polynucleotide.

As one example, through the combined use of CRISPR-Cas9 live-cell genomic imaging and CRISPR-GO, relocalization of endogenous genomic loci to the nuclear periphery has been shown to occur in both a mitosis-dependent and -independent manner. During mitosis, the nuclear membrane breaks down in prometaphase and then reforms in telophase. The dramatic changes in chromatin and nuclear structure during mitosis could facilitate interactions between genomic loci and the nuclear membrane to create nuclear envelope tethering. During interphase, though chromatin structure remains relatively stable, a genomic locus can still form interactions with the nuclear periphery when it is in close proximity. Nuclear periphery tethering during interphase may rely on proximity between the targeted loci and nuclear periphery, and a genomic locus that is located distal to the nuclear periphery may less likely be tethered through the mitosis-independent manner.

The chemical induction process of some provided embodiments also allows for the investigation of the real-time association between a target polynucleotide locus and cellular compartments in living cells. For example, compared to the relatively slower repositioning to the nuclear periphery (within hours), colocalization between a genomic locus and Cajal bodies occurs at a much faster rate (within minutes), likely because Cajal body components are more diffuse throughout the nucleus. Using the disclosed systems and methods, it has been observed that colocalization between CBs and the target genomic loci could occur in two ways: one is rapid formation of de novo Cajal bodies at the genomic loci, and the other is re-localization of existing CBs with the target genomic loci, a phenomenon which has not been reported before. Previous work has suggested that Cajal bodies are formed by phase separation. The recruiting of nuclear body components (e.g., Coilin for CBs) by CRISPR-GO to targeted genomic loci may generate synthetic phase separation at the target chromatin loci.

The provided compositions, methods, and systems have also been used to observe repression of an adjacent fluorescent reporter gene when repositioning a genomic locus to the nuclear periphery. Previous work reported different effects on gene expression after tethering LacO loci to the nuclear periphery. In particular, earlier studies have observed no change in transcription after LacO repeats were recruited to the nuclear periphery by LacI-Lamin B, and have shown that tethering LacO repeats to nuclear periphery by LacI-Emerin caused repression of adjacent genes. The systems disclosed herein have shown that repositioning the reporter gene to Emerin causes gene repression (˜59%).

The systems, compositions, and methods disclosed herein have also been used to repress both adjacent reporter and endogenous genes after CRISPR-GO-mediated colocalization of a chromatin locus to CBs. Importantly, targeted colocalization of Cajal bodies with endogenous loci represses adjacent gene expression across long distances (>30 kb). This observed gene repression after targeting a genomic locus to CBs has not yet been reported. In contrast, the CRISPRi/a methods function by recruiting transcriptional effectors that mostly affect expression of local genes within a few kilobases around the target site. Thus, the provided methods and systems provide an important new method for regulating polynucleotide expression over a long distance. The methods and systems also provide the ability to control repositioning of target polynucleotides to diverse cellular compartments in a systematic way to investigate cellular effects and program polynucleotide regulation.

The systems, compositions, and methods disclosed herein have also been used to regulate both proximal genes and distal genes after CRISPR-GO-mediated formation of a heterochromatin to a target polynucleotide, such as region on a chromosome comprising tandem repeats. Importantly, targeted formation of heterochromatin with endogenous loci can regulate gene expression across a region comprising a target polynucleotide. This can include regulation of expression of genes that are proximal to the target polynucleotide or distal to the target polynucleotide. Thus, the provided methods and systems provide an important new method for regulating polynucleotide expression over both short and long distances. The methods and systems also provide the ability to control positioning of the formation of the compartment, e.g., heterochromatin compartment, by targeting a specific polynucleotide.

### Compartment Formation

In some embodiments, the systems, compositions, and methods disclosed herein are used with endogenous or synthetic oligomerizing proteins that self-aggregate to form an artificial protein/RNA/DNA aggregate, which can possess one or more unique chemical, physical, or biological properties (such as selective diffusion of specific proteins, RNA, or DNA; association or disassociation with other molecules; promotion or inhibition of gene regulation machineries; or promotion or inhibition of DNA recombination or stability machineries). Such an aggregate is a compartment that can be formed around target polyribonucleotide and is referred to herein as a synthetic cellular phases (SCP). These aggregates can have strong effects on gene regulation or polynucleotide repair mechanisms. The formation of these aggregrates can additionally be used for introducing epigenetic modifications to, producing three-dimensional structures, a topologically associating domains, or genomic boundaries comprising the target polynucleotide or an additional polynucleotide (e.g., distal or proximal gene from the target polynucleotide). In some embodiments, a protein, protein domain, RNA, RNA domain, or combination thereof is coupled to a provided system to specifically form a desired SCP around desired chromatin DNA or RNA. For example, proteins that facilitate HDR are used to form a SCP around a target polynucleotide that has been or will be cut by a gene editing technique, such as by CRISPR/Cas, TALENs, ZFNs, or meganucleases, or other polynucleotide cutting mechanism. Exemplary proteins that facilitate HDR can comprise Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. Additionally, the SCP that facilitates HDR can comprise a template polynucleotide. For example, proteins that facilitate heterochromatin are used to form a SCP around a target polynucleotide to regulate gene expression of polynucleotides within a genomic region comprising the target polynucleotide. Exemplary proteins that facilitate this heterochromatin SCP are HP1α, HP1β, KAP1, KRAB, SUV39H1, or G9a. In some embodiments, the provided systems, compositions, and methods are useful for manipulating the spatiotemporal organization of genomic DNA and RNA components in the nucleus and/or cytoplasm and for regulating diverse cellular functions.

The systems, compositions, and methods as disclosed herein can be used to control a local environment of a target polynucleotide. This can be accomplished by spatial positioning of proteins to the target polynucleotide. In one aspect, a system is provided for spatial positioning of compartment-constituent protein to a target polynucleotide. The compartment-constituent protein can comprise further oligomerization domains or other domains that recruit additional compartment constituent proteins to the target polynucleotide to form the compartment, such as an SCP. In some embodiments, a method of forming a compartment around a target polynucleotide, the method comprising: (a) providing a compartment-constituent protein linked to a first dimerization domain; (b) providing an actuator moiety linked to a second dimerization domain, wherein the actuator moiety and the target polynucleotide form a complex; and (c) assembling a dimer comprising the first dimerization domain and the second dimerization domain of the complex, thereby forming the compartment around the target polynucleotide. For example, the compartment-constituent protein is a heterochromatin protein. The system can comprise a heterochromatin protein linked to a first dimerization domain. The system further can comprise an actuator moiety that targets the target polynucleotide, wherein the actuator moiety is linked to a second dimerization domain that is capable of assembling into a dimer with the dimerization domain. The assembly of the dimer can result in the positioning of the heterochromatin protein to the target polynucleotide. In some embodiments, the compartment is formed by administering a composition as described herein. A composition can comprise a) a compartment-constituent protein linked to a first dimerization domain; and b) an actuator moiety linked to a second dimerization domain; and wherein the first dimerization domain binds to the second dimerization domain. In some embodiments, the compartment is formed by a composition comprising a) a compartment-constituent protein linked to a first dimerization domain; and b) an actuator moiety linked to a scaffold, wherein the scaffold is linked to a second dimerization domain; and wherein the first dimerization domain binds to the second dimerization domain. In some embodiments, the composition further comprises a ligand, wherein the first dimerization domain and the second dimerization domain bind to the ligand, thereby linking the first dimerization domain to the second dimerization domain. In some embodiments, the ligand is inducible. In some embodiments, the scaffold is linked to at least 1, 2, 3, 4, 5, 6, 7, 8, 9, or 10 second dimerization domains. In some embodiments, the scaffold is linked to more than 1, 2, 3, 4, 5, 6, 7, 8, 9, or 10 second dimerization domains. In some embodiments, the scaffold is linked to 1, 2, 3, 4, 5, 6, 7, 8, 9, or 10 second dimerization domains. In some embodiments, the compartment-constituent protein is further linked to a second scaffold, wherein the second scaffold is linked to at least one first dimerization domain. In some embodiments, the second scaffold is linked to at least 1, 2, 3, 4, 5, 6, 7, 8, 9, or 10 first dimerization domains. In some embodiments, the second scaffold is linked to more than 1, 2, 3, 4, 5, 6, 7, 8, 9, or 10 first dimerization domains. In some embodiments, the second scaffold is linked to 1, 2, 3, 4, 5, 6, 7, 8, 9, or 10 second first domains.

In some embodiments, the systems, compositions, and methods comprise an inducible dimerization, wherein the dimerization can be mediated chemically or optogenetically. The dimer can be a homodimer. The dimer can be a heterodimer In some embodiments, the dimerization is mediated by a molecular ligand, such as a chemical inducer. In certain aspects, the dimerization system is selected from an ABA induced ABI/PYL1 dimerization system, a GA induced GA/GAI dimerization system a Rapamycin induced FRB/FKBP dimerization system a TMP-Htag induced HaloTag/DHFR dimerization system, or a dimerization system using an enzyme-catalyzed reaction. Other dimerization systems are also contemplated.

In some embodiments, the targeted polynucleotide of the provided systems and methods comprises DNA, e.g., genomic DNA. In some embodiments, the target polynucleotide comprises RNA, e.g., mRNA, microRNA, siRNA, or non-coding RNA. Actuator moieties and related targeting systems suitable for use with the provided systems and methods include, for example, CRISPR-Cas (including all types of CRISPR, type I, II, III, IV, V, VI, e.g., Cas9, Cas12, Cas13); Argonaute-mediated targeting or zinc finger targeting; TALE (transcription activator-like effectors); LacO-LacI or TetO-TetR; and specific pairs of DNA interacting protein or RNA domains. Cas9 and Cas13 can also target RNA in a sequence-dependent way, and can be used in this way with the provided system to re-localize RNA molecules to different cellular compartments. Cas proteins can lack DNA cleavage activity. The targeting systems can include sequence-specific guide RNAs or guide DNAs.

The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease, a derivative thereof, a variant thereof, or a fragment thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity. An actuator moiety having reduced or minimal nuclease activity can regulate expression and/or activity of a gene by physical obstruction of a target polynucleotide or recruitment of additional factors effective to suppress or enhance expression of the target polynucleotide. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety. An actuator moiety can regulate expression or activity of a gene and/or edit a nucleic acid sequence, whether exogenous or endogenous.

Any suitable nuclease can be used in an actuator moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof and any fragment thereof.

In some embodiments, the actuator moiety comprises a CRISPR-associated (Cas) protein or a Cas nuclease which functions in a non-naturally occurring CRISPR (Clustered Regularly Interspaced Short Palindromic Repeats)/Cas (CRISPR-associated) system. In bacteria, this system can provide adaptive immunity against foreign DNA (Barrangou, R., et al, “CRISPR provides acquired resistance against viruses in prokaryotes,” Science (2007) 315: 1709-1712; Makarova, K. S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39: 9275-9282).

In a wide variety of organisms including diverse mammals, animals, plants, microbes, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E286; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (2011) 471:602-607), and has been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31: 233-239; Sander, J. D. & Joung, J. K, “CRISPR-Cas systems for editing, regulating and targeting genomes,” Nature Biotechnol. (2014) 32:347-355).

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity. An actuator moiety comprising a Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products. A Cas protein can edit a nucleic acid sequence by generating a double-stranded break or single-stranded break in a target polynucleotide. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, the actuator moiety comprises a Cas protein that forms a complex with a guide nucleic acid, such as a guide RNA. In some embodiments, the actuator moiety comprises a Cas protein that forms a complex with a single guide nucleic acid, such as a single guide RNA (sgRNA). In some embodiments, the actuator moiety comprises a RNA-binding protein (RBP) optionally complexed with a guide nucleic acid, such as a guide RNA (e.g., sgRNA), which is able to form a complex with a Cas protein. In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA.

Any suitable CRISPR/Cas system can be used. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpfl, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, HID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

An actuator moiety comprising a Cas protein can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, CaslOd, Cas10, CaslOd, CasF, CasG, CasH, Cpfl, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromo genes, Streptosporangiurn roseum, Streptosporangiurn roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobiurn arabaticum, Ammomfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatiurn vinosum, Marinobacter sp., Nitrosococcus halophilus, Nitrosococcus watsoni, Pseudoalteromonas haloplanktis, Ktedonobacter racemifer, Methanohalobiurn evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacteriurn mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. torquens, Ilyobacter polytropus, Ruminococcus albus, Akkermansia mucimphila, Acidothermus cellulolyticus, Bifidobacterium longum, Bifidobacterium dentium, Corynebacterium diphtheria, Elusimicrobiurn minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like 20 nuclease domain. The RuvC and HNH domains can each cut a different strand of double-stranded DNA to make a double-stranded break in the DNA. A Cas protein can comprise only one nuclease domain (e.g., Cpfl comprises RuvC domain but lacks HNH domain).

A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

A Cas protein can be a fusion protein. For example, a Cas protein can be fused to a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

A Cas protein can be provided in any form. For example, a Cas protein can be provided in the form of a protein, such as a Cas protein alone or complexed with a guide nucleic acid. A Cas protein can be provided in the form of a nucleic acid encoding the Cas protein, such as an RNA (e.g., messenger RNA (mRNA)) or DNA. The nucleic acid encoding the Cas protein can be codon optimized for efficient translation into protein in a particular cell or organism.

Nucleic acids encoding Cas proteins can be stably integrated in the genome of the cell. Nucleic acids encoding Cas proteins can be operably linked to a promoter active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein is a dead Cas protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

A dCas9 polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell. The nucleic acids targeted by the guide RNA can be any that are expressed in a cell such as an immune cell. The nucleic acids targeted may be a gene involved in immune cell regulation. In some embodiments, the nucleic acid is associated with cancer. The nucleic acid associated with cancer can be a cell cycle gene, cell response gene, apoptosis gene, or phagocytosis gene. The recombinant guide RNA can be recognized by a CRISPR protein, a nuclease-null CRISPR protein, variants thereof, or derivatives thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a wild-type exemplary activity (e.g., nucleic acid cleaving activity, wild-type Cas9 activity).

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpfl protein) are deleted or mutated, the resulting Cas protein can have a reduced or no ability to cleave both strands of a double-stranded DNA. An example of a mutation that can convert a Cas9 protein into a nickase is a D 10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A dead Cas protein can comprise one or more mutations relative to a wild-type version of the protein. The mutation can result in no more than 90%, no more than 80%, le no more ss than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D 10A, G12A, G17A, E762A, H840A, N854A, N863A, H982A, H983A, A984A, and/or D986A. Mutations other than alanine substitutions can be suitable.

A D1OA mutation can be combined with one or more of H840A, N854A, or N856A mutations to produce a Cas9 protein substantially lacking DNA cleavage activity (e.g., a dead Cas9 protein). A H840A mutation can be combined with one or more of D1OA, N854A, or N856A mutations to produce a site-directed polypeptide substantially lacking DNA cleavage activity. A N854A mutation can be combined with one or more of H840A, D1OA, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. aureus. In some embodiments, the Cas9 protein is derived from a Cas9 protein from S. pyogenes or S. aureus. For example, a S. pyogenes or S. aureus Cas9 protein lacking cleavage activity.

Cas9 can generally refer to a polypeptide with at least about 5%, 10%, 20%, 30%, 40%, 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In some embodiments, an actuator moiety comprises a “zinc finger nuclease” or “ZFN.” ZFNs refer to a fusion between a cleavage domain, such as a cleavage domain of Fokl, and at least one zinc finger motif (e.g., at least 2, 3, 4, or 5 zinc finger motifs) which can bind polynucleotides such as DNA and RNA. The heterodimerization at certain positions in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Publication Nos. 2003/0232410 and 2009/0203140.

In some embodiments, an actuator moiety comprising a ZFN can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. In some embodiments, a ZFN is a zinc finger nickase which induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33. In some embodiments, a ZFN binds a polynucleotide (e.g., DNA and/or RNA) but is unable to cleave the polynucleotide.

In some embodiments, the cleavage domain of an actuator moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have no more than 90%, no more than than 80%, no more than 70%, no more than 60%, no more than 50%, no more than 40%, no more than 30%, no more than 20%, no more than 10%, no more than 5%, or no more than 1% of the nucleic acid-cleaving activity of the wild-type cleavage domain. The modified form of the cleavage domain can have no substantial nucleic acid-cleaving activity. In some embodiments, the cleavage domain is enzymatically inactive.

In some embodiments, an actuator moiety comprises a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fokl endonuclease or the catalytic domain of Fokl. Several mutations to Fokl have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. In some cases, a single-stranded donor DNA repair template is provided to promote HDR. Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. A non-limiting example of a transcriptional repression domain includes a Kruppel-associated box domain.

In some embodiments, an actuator moiety comprises a meganuclease. Meganucleases generally refer to rare-cutting endonucleases or homing endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A meganuclease can generate a double-stranded break. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof. Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

In some embodiments, the nuclease domain of a meganuclease comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

In some embodiments, the actuator moiety is fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the enzyme. In other cases, the actuator moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-KB p65 subunit, Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Kruppel-associated box) domain of Koxl, the Mad mSIN3 interaction domain (SID), ERF repressor domain (ERD), and are described in Chavez et al., Nat Methods, 2015, 12(4):326-328 and U.S. Patent App. Publ. No. 20140068797. An actuator moiety can also be fused to a heterologous polypeptide providing increased or decreased stability. The fused domain or heterologous polypeptide can be located at the N-terminus, the C-terminus, or internally within the actuator moiety.

An actuator moiety can comprise a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g. eBFP, eBFP2, Azurite, mKalamal, GFPuv, Sapphire, T-sapphire), cyan fluorescent proteins (e.g. eCFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

In some embodiments, the actuator moiety and the second dimerization domain are linked via a linker. A linker can be any linker known in the art. In some embodiments, the actuator moiety and second dimerization domain are linked as fusion protein. In some embodiments, the compartment-constituent protein and the first dimerization domain are linked via a linker. A linker can be any linker known in the art. In some embodiments, the compartment-constituent protein and the first dimerization domain are linked as fusion protein.

In some embodiments, the target polynucleotide is positioned by the provided systems and methods in an inner nuclear membrane. Compartment specific proteins suitable for targeting the inner nuclear membrane include, but are not limited to, Emerin, Lap2beta, and Lamin B.

In some embodiments, the target polynucleotide is positioned by the provided systems and methods in a Cajal body. In some embodiments, the Cajal body is assembled or positioned by the provided systems and methods at the target polynucleotide. Suitable compartment specific proteins for Cajal bodies for the provided systems and methods include, but are not limited to, Coilin, SMN, Gemin 3, SmD1, and SmE.

In some embodiments, the target polynucleotide is positioned by the provided systems and methods in nuclear speckles. In some embodiments, the nuclear speckle is assembled or positioned by the provided systems and methods at the target polynucleotide. Suitable compartment specific proteins for nuclear speckles for the provided systems and methods include, but are not limited to, SC35.

In some embodiments, the target polynucleotide is positioned by the provided systems and methods in a PML body. In some embodiments, the PML body is assembled or positioned by the provided systems and methods at the target polynucleotide. Suitable compartment specific proteins for PML bodies for the provided systems and methods include, but are not limited to, PML and SP100.

In some embodiments, the target polynucleotide is positioned by the provided systems and methods in a nuclear pore complex. Compartment specific proteins suitable for targeting nuclear pore complexes include, but are not limited to, Nup50, Nup98, Nup53, Nup153, and Nup62.

In some embodiments, the target polynucleotide is positioned by the provided systems and methods in a nucleolus. Compartment specific proteins suitable for targeting the nucleolus include, but are not limited to, nuclear protein B23.

In some embodiments, the target polynucleotide is positioned by the provided systems and methods in a P granule. In some embodiments, P granule is assembled or positioned by the provided systems and methods at the target polynucleotide. Suitable compartment specific proteins for P granules for the provided systems and methods include, but are not limited to, RGG domain proteins, PGL-1 and PGL-3; Dead box proteins, and GLH-1-4.

In some embodiments, the target polynucleotide is positioned by the provided systems and methods in a GW body. In some embodiments, the GW body is assembled or positioned by the provided systems and methods at the target polynucleotide. Suitable compartment specific proteins for GW bodies for the provided systems and methods include, but are not limited to, GW182.

In some embodiments, the target polynucleotide is positioned by the provided systems and methods in a stress granule. In some embodiments, the stress granule is assembled or positioned by the provided systems and methods at the target polynucleotide. Suitable compartment specific proteins for stress granules for the provided systems and methods include, but are not limited to, G3BP (Ras-GAP SH3 binding proteins), TIA-1 (T-cell intracellular antigen), eIF2, and eIF4E.

In some embodiments, the target polynucleotide is positioned by the provided systems and methods in a sponge body. In some embodiments, the sponge body is assembled or positioned by the provided systems and methods at the target polynucleotide. Suitable compartment specific proteins for sponge bodies for the provided systems and methods include, but are not limited to, EXu, Btz, Tral, Cup, eIF4E, Me31B, Yps, Gus, Dcp1/2, Sqd, BicC, Hrb27C, and Bru.

In some embodiments, the target polynucleotide is positioned by the provided systems and methods in a cytoplasmic prion protein induced ribonucleoprotein (CyPrP-RNP) granules. In some embodiments, the CyPrP-RNP is assembled or positioned by the provided systems and methods at the target polynucleotide. Suitable compartment specific proteins for CyPrP-RNP granules for the provided systems and methods include, but are not limited to, Dcpla, DDX6/Rck/p54/Me31B/Dhhl, and Dicer.

In some embodiments, the target polynucleotide is positioned by the provided systems and methods in a U body. In some embodiments, the U body is assembled or positioned by the provided systems and methods at the target polynucleotide. Suitable compartment specific proteins for U bodies for the provided systems and methods include, but are not limited to, uridine-rich small nuclear ribonucleoproteins U1, U2, U4/U6 and U5; LSm1-7; and the survival of motor neurons (SMN) protein.

In some embodiments, the target polynucleotide is positioned by the provided systems and methods in the endoplasmic reticulum. Compartment specific proteins suitable for targeting the endoplasmic reticulum include, but are not limited to, Calreticulin, Calnexin, PDI, GRP 78, and GRP 94.

In some embodiments, the target polynucleotide is positioned by the provided systems and methods in a mitochondrium. Compartment specific proteins suitable for targeting mitochondria include, but are not limited to, HIF1A, PLN, Cox 1, Hexokinase, and TOMM40.

In some embodiments, the target polynucleotide is positioned by the provided systems and methods in the plasma membrane. Compartment specific proteins suitable for targeting the plasma membrane include, but are not limited to, sodium potassium ATPase, CD98, Cadherins, and plasma membrane calcium ATPase (PMCA).

In some embodiments, the target polynucleotide is positioned by the provided systems and methods in golgi. Compartment specific proteins suitable for targeting golgi include, but are not limited to, GM130, MAN2A1, MAN2A2, GLG1, B4GALT1, RCAS1, and GRASP65.

In some embodiments, the target polynucleotide is positioned by the provided systems and methods in a ribosome. In some embodiments, the ribosome is assembled or positioned by the provided systems and methods at the target polynucleotide. Suitable compartment specific proteins for ribosomes for the provided systems and methods include, but are not limited to, AGO2, MTOR, PTEN, RPL26, FBL, and RPS3.

In some embodiments, the target polynucleotide is positioned by the provided systems and methods in a proteasome. In some embodiments, the proteasome is assembled or positioned by the provided systems and methods at the target polynucleotide. Suitable compartment specific proteins for proteasomes for the provided systems and methods include, but are not limited to, PSMA1, PSMBS, PSMC1, PSMD1, and PSMD7.

In some embodiments, the target polynucleotide is positioned by the provided systems and methods in an endosome. Compartment specific proteins suitable for targeting endosomes include, but are not limited to, CFTR, ADRB1, EGFR, IGF2R, AP2S1, CD4, HLA-A, Coveolin, RABS, and ErbB2.

In some embodiments, the target polynucleotide is positioned by the provided systems and methods in a liposome. Compartment specific proteins suitable for targeting liposomes include, but are not limited to, EEA1, LAMTOR2, and LAMTOR4.

In some embodiments, the target polynucleotide is positioned by the provided systems and methods in a synthetic cellular phase. In some embodiments, a synthetic cellular phase is positioned by the provided systems and methods by a target polynucleotide. A synthetic cellular phase can facilitate HDR. Suitable compartment-constituent proteins for facilitating HDR include, but are not limited to Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. A synthetic cellular phase can facilitate heterochromatin. Suitable compartment-constituent proteins for facilitating heterochromatin include, but are not limited to, HP1α, HP1(3, KAP1, KRAB, SUV39H1, or G9a.

Other cell compartments that can be targeted with the systems and methods disclosed herein include RNP bodies, mitotic spindles, histone locus bodies, heterochromatin regions, and the cytoskeleton. Additional compartments are also contemplated.

The target polynucleotide can be endogenous or exogenous to the cell compartment to which it is positioned. The target polynucleotide can be endogenous or exogenous to the cell. The target polynucleotide can be human or non-human. The target polynucleotide can be virally derived, a plasmids, a ribonucleoprotein, or a synthesized RNA or DNA strand.

The methods and systems disclosed herein are suitable for use in multiplexed processes in which multiple polynucleotides are repositioned to the same or different cellular compartments.

In some embodiments, the provided systems and methods are used to mediate de novo cellular compartment (e.g., nuclear body) formation at targeted polynucleotide (e.g., genomic) loci, providing a potential method to initiate membraneless organelle formation via liquid-liquid phase separation. Membraneless organelle or compartment assembly can be used to create specific environments around a polynucleotide, such an environment that facilitates polynucleotide repair. For example, a compartment is assembled around a target polynucleotide that has been cut using a gene editing technique, and this compartment comprises a template polynucleotide and Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. For example, a compartment is assembled around a target polynucleotide to regulate gene expression, and this compartment comprises HP1α, HP1(3, KAP1, KRAB, SUV39H1, or G9a. Membraneless compartmentalization of the subcellular space occurs by liquid-liquid phase separation. Heterotypic cooperative weak interactions enable rapid rearrangements within liquid compartments. Intrinsically disordered proteins play important roles in phase transitions due to their structural plasticity and prion-like properties. Cells dynamically control the extent and duration of phase transitions. Molecular seeds such as DNA, RNA or poly(ADP-ribose) (PAR) can trigger phase transitions in a stimulus- and context-specific manner. Chaperones, disintegrase machineries, and post-translational modifications cooperate to control phase transitions. A continuum of aggregation propensities exists and cells employ an unanticipated broad range of material states in proteinaceous assemblies. These can progress into pathological aggregates associated with neurodegenerative diseases.

Examples of synthetic phases that can be formed using the systems and methods disclosed herein include, but are not limited to, synthetic PML bodies that can have roles in viral defense and telomere maintenance, synthetic nuclear speckles and paraspeckles that can be stress inducible anti-apoptotic structures, synthetic gems that can be hubs for factors involved in neurodegeneration, synthetic architectural RNAs that can seed nuclear bodies, synthetic nucleoli, synthetic heterochromatin or euchromatin, synthetic histone locus bodies that can be sites of FLASH accumulation and enhance histone mRNA processing, synthetic chromatin packing systems that can involve the use of Xist to silence in cis the whole chromosome, synthetic epigenetic phases, synthetic (cytoplasmic) P bodies, synthetic stress bodies, synthetic germ granules that can generate sexual cells upon meiosis in the developing embryo, synthetic mRNP granules in neurodegenerative disease, synthetic posttranslational modifications (PTM) that can regulate membrane-less organelle structure and dynamics, synthetic IDP (intrinsically disordered proteins) forming aggregates, synthetic prion like domains (PLDs) or RGG-rich low-complexity domains (LCD), and synthetic polynucleotide repair bodies. For example, a synthetic polynucleotide repair body can be a synthetic HDR body that comprises a template polynucleotide and proteins that facilitate HDR such as Rad51, Rad52, RPA, MRN complex, MRX complex, CtlP, Sae2, BLM, Sgs1, BRCA2, exonucleases such as Exo1 or OsExo1, ATM, BRCA2, RAD54, or MDC1. For example, a synthetic gene regulation body can be a synthetic heterochromatin body that comprises protein that facilitate gene regulation such as HP1α, HP1β, KAP1, KRAB, SUV39H1, or G9a. Other non-endogenous protein/RNA aggregates to which polynucleotides can be positioned include β-amyloid bodies, mRNA aggregates, Xist packaging complexes, and others.

The controlled positioning of polynucleotides or compartments as describe herein can be used to regulate, modify, or influence, for example, DNA interaction with RNA Polymerases, transcription factors, pioneer factors, mediators, DNA looping molecules, and other DNA associated proteins; epigenetic modification marks or euchromatin/heterochromatin modulating enzymes (e.g., HP1); chromatin compactness and other biophysics/biochemical properties; gene editing, including recombination, NHEJ, or HDR; genome stability and cancer; DNA repair processes; and mRNA metabolism through splicing, degradation, translation, methylation, localization, and interaction with other chaperons and RNA-binding proteins.

The methods, compositions, and systems disclosed herein can be used to establish inducible and reversible disease models to understand disease mechanism. For example, the provided systems and methods can be used to investigate diseases caused by protein/RNA misfolding or aggregations. Proteome imbalances are associated with aging and often involve abundant proteins that exceed solubility and tend to form intracellular and extracellular aggregates. Aging is a risk factor for the onset of several protein misfolding disorders (PMDs), particularly for progressive neurodegeneration. Protein aggregation is the primary hallmark of neurodegeneration, including amyloid beta (Ab) and tau aggregation in Alzheimer's disease (AD), intracellular alpha-synuclein aggregates in Parkinson's disease (PD) and multisystem atrophy, polyQ-driven protein aggregates in Huntington's disease (HD), PrPSc in prion diseases, and TDP-43 and FET protein aggregates in amyotrophic lateral sclerosis (ALS) and frontotemporal dementia (FTD), just to list a few examples. Although the chemical nature and the (patho)physiological topology of the proteins involved in plaque formation differ, the principles that govern their aggregation appear surprisingly similar, and the provided methods and systems can be used to position target polynucleotides at these plaques or aggregates.

The systems, compositions, and methods disclosed herein can be used to control cell differentiation by repositioning key drivers genes into different nuclear compartments. The systems and methods can be used to enhance antibody production by controlling the recombination rate at the endogenous VD(J) locus. The systems and methods can be used for mitigating Alzheimer's by eliminating the formation of misfolding protein bodies.

The systems, compositions, and methods disclosed herein can be used to co-localize or assemble a compartment at a target polynucleotide or gene locus. By repositioning or forming the compartment, the location of the target polynucleotide or gene locus is maintained within the cell, and thus allowing for the compartment to impact the target polynucleotide or gene locus without more broadly impacting the position of the chromatin and the functions associated with the positioning of the chromatin. The systems and methods can be used enhance or facilitate polynucleotide repair mechanisms, such as HDR, recombination, or NHEJ, by assembling compartments comprising proteins that facilitate the polynucleotide repair mechanism around a target polynucleotide.

The systems, compositions, and methods disclosed herein are broadly applicable in all kingdoms of life, including plants, bacteria, archaea, yeast, fishes, insects, birds, mammals, mice, pigs, and humans. The systems and methods can be used in living whole organisms or in tissue or cells.

The systems, compositions, and methods disclosed herein can be used to form compartments around a target polynucleotide that can be used for regulating expression of (e.g., increasing or decreasing), introducing epigenetic modifications to, producing three-dimensional structures, a topologically associating domains, or genomic boundaries comprising the target polynucleotide or an additional polynucleotide (e.g., distal or proximal gene from the target polynucleotide).

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A system for controlling the spatial and temporal positioning of a target polynucleotide in a compartment of a cell, the system comprising: (a) a compartment-specific protein linked to a first dimerization domain; and (b) an actuator moiety that targets the target polynucleotide, wherein the actuator moiety is linked to a second dimerization domain that is capable of assembling into a dimer with the first dimerization domain. 2. The system of embodiment 1, wherein the target polynucleotide comprises genomic DNA. 3. The system of embodiments 1, wherein the target polynucleotide comprises RNA. 4. The system of any one of embodiments 1-3, wherein the actuator moiety comprises a Cas protein, and wherein the system further comprises: (c) a guide RNA that complexes with the actuator moiety and hybridizes to the target polynucleotide. 5. The system of any one of embodiments 1-3, wherein the actuator moiety comprises an RNA binding protein complexed with a guide RNA that hybridizes to the target polynucleotide, and wherein the system further comprises: (c) a Cas protein that complexes with the guide RNA. 6. The system of embodiment 4 or 5, wherein the Cas protein substantially lacks DNA cleavage activity. 7. The system of any one of embodiments 4-6, wherein the Cas protein is a Cas9 protein, a Cas12 protein, a Cas13 protein, a CasX protein, or a CasY protein. 8. The system of embodiment 7, wherein the Cas12 protein is selected from the group consisting of Cas12a, Cas12b, Cas12c, Cas12d, and Cas12e. 9. The system of any one of embodiments 1-3, wherein the actuator moiety comprises a binding protein that hybridizes to the target polynucleotide, wherein the binding protein is a zinc finger nuclease or a TALE nuclease. 10. The system of any one of embodiments 1-3, wherein the actuator moiety comprises an Argonaute protein complexed with a guide polynucleotide, wherein the guide polynucleotide is a guide RNA or a guide DNA, and wherein the guide polynucleotide hybridizes to the target polynucleotide. 11. The system of any one of embodiments 1-10, wherein the compartment is a nuclear compartment. 12. The system of embodiment 11, wherein the nuclear compartment comprises an inner nuclear membrane. 13. The system of embodiment 12, wherein the compartment-specific protein comprises Emerin, Lap2beta, Lamin B, or a combination thereof 14. The system of embodiment 11, wherein the nuclear compartment comprises a Cajal body. 15. The system of embodiment 14, wherein the compartment-specific protein comprises coilin, SMN, Gemin 3, SmD1, SmE, or a combination thereof 16. The system of embodiment 11, wherein the nuclear compartment comprises a nuclear speckle. 17. The system of embodiment 16, wherein the compartment-specific protein comprises SC35. 18. The system of embodiment 11, wherein the nuclear compartment comprises a PML body. 19. The system of embodiment 18, wherein the compartment-specific protein comprises PML, SP100, or a combination thereof 20. The system of embodiment 11, wherein the nuclear compartment comprises a nuclear core complex. 21. The system of embodiment 20, wherein the compartment-specific protein comprises Nup50, Nup98, Nup53, Nup153, Nup62, or a combination thereof. 22. The system of embodiment 11, wherein the nuclear compartment comprises a nucleolus. 23. The system of embodiment 22, wherein the compartment-specific protein comprises nucleolar protein B23. 24. The system of any one of embodiments 1-10, wherein the compartment is a cytosolic compartment. 25. The system of any one of embodiments 1-24, wherein the compartment-specific protein is further linked to a fluorescent protein. 26. The system of any one of embodiments 1-25, wherein the actuator moiety is further linked to a fluorescent protein. 27. The system of any one of embodiments 1-27, wherein the first dimerization domain and the second dimerization domain assemble to form a dimer only in the presence of a ligand. 28. The system of embodiment 27, wherein the first dimerization domain and the second dimerization domain each bind to the ligand in the presence of the ligand. 29. The system of embodiment 27 or 28, wherein the ligand is a chemical inducer. 30. A method of controlling the spatial and temporal positioning of a target polynucleotide in a compartment of a cell, the method comprising: (a) providing a compartment-specific protein linked to a first dimerization domain; (b) providing an actuator moiety linked to a second dimerization domain; (c) forming a complex comprising the actuator moiety and the target polynucleotide; and (d) assembling a dimer comprising the first dimerization domain and the second dimerization domain, thereby positioning the target polynucleotide in the compartment. 31. The method of embodiment 30, wherein the target polynucleotide is not endogenous to the compartment. 32. The method of embodiment 30 or 31, wherein the positioning of the target polynucleotide comprises regulating the expression of the target polynucleotide. 33. The method of embodiment 32, wherein the regulating comprises decreasing the expression of the target polynucleotide. 34. The method of embodiment 32, wherein the regulating comprises increasing the expression of the target polynucleotide. 35. The method of any one of embodiments 30-34, wherein the positioning of the target polynucleotide further comprises regulating the expression of one or more additional polynucleotides endogenous to the compartment. 36. The method of any one of embodiments 30-35, wherein the positioning of the target polynucleotide further comprises creating one or more additional compartments within the cell. 37. The method of any one of embodiments 30-36, wherein the positioning of the target polynucleotide further comprises repairing a DNA break. 38. The method of embodiment 41, wherein the repairing comprises introducing exogenous DNA. 39. The method of embodiment 42, wherein the introducing comprises recombination, non-homologous end-joining, or homology-directed repair. 40. The method of any one of embodiments 30-39, wherein the positioning of the target polynucleotide further comprises creating an artificial aggregate, wherein the artificial aggregate comprises protein, RNA, DNA, or a combination thereof 41. The method of any one of embodiments 30-40, wherein the target polynucleotide comprises genomic DNA. 42. The method of any one of embodiments 30-40, wherein the target polynucleotide comprises RNA. 43. The method of any one of embodiments 30-42, wherein the actuator moiety comprises a Cas protein, and wherein the system further comprises: (c) a guide RNA that complexes with the actuator moiety and hybridizes to the target polynucleotide. 44. The method of any one of embodiments 30-42, wherein the actuator moiety comprises an RNA binding protein complexed with a guide RNA that hybridizes to the target polynucleotide, and wherein the system further comprises: (c) a Cas protein that complexes with the guide RNA. 45. The method of embodiment 43 or 44, wherein the Cas protein substantially lacks DNA cleavage activity. 46. The method of any one of embodiments 43-45, wherein the Cas protein is a Cas9 protein, a Cas12 protein, a Cas13 protein, a CasX protein, or a CasY protein. 47. The method of embodiment 46, wherein the Cas12 protein is selected from the group consisting of Cas12a, Cas12b, Cas12c, Cas12d, and Cas12e. 48. The method of any one of embodiments 30-42, wherein the actuator moiety comprises a binding protein that hybridizes to the target polynucleotide, wherein the binding protein is a zinc finger nuclease or a TALE nuclease. 49. The method of any one of embodiments 30-42, wherein the actuator moiety comprises an Argonaute protein complexed with a guide polynucleotide, wherein the guide polynucleotide is a guide RNA or a guide DNA, and wherein the guide polynucleotide hybridizes to the target polynucleotide. 50. The method of any one of embodiments 30-49, wherein the compartment is a nuclear compartment. 51. The method of embodiment 50, wherein the nuclear compartment comprises an inner nuclear membrane. 52. The method of embodiment 51, wherein the compartment-specific protein comprises Emerin, Lap2beta, Lamin B, or a combination thereof 53. The method of embodiment 50, wherein the nuclear compartment comprises a Cajal body. 54. The method of embodiment 53, wherein the compartment-specific protein comprises coilin, SMN, Gemin 3, SmD1, SmE, or a combination thereof 55. The method of embodiment 50, wherein the nuclear compartment comprises a nuclear speckle. 56. The method of embodiment 55, wherein the compartment-specific protein comprises SC35. 57. The method of embodiment 50, wherein the nuclear compartment comprises a PML body. 58. The method of embodiment 57, wherein the compartment-specific protein comprises PML, SP100, or a combination thereof 59. The method of embodiment 50, wherein the nuclear compartment comprises a nuclear core complex. 60. The method of embodiment 59, wherein the compartment-specific protein comprises Nup50, Nup98, Nup53, Nup153, Nup62, or a combination thereof 61. The method of embodiment 50, wherein the nuclear compartment comprises a nucleolus. 62. The method of embodiment 61, wherein the compartment-specific protein comprises nucleolar protein B23. 63. The method of any one of embodiments 30-49, wherein the compartment is a cytosolic compartment. 64. The method of any one of embodiments 30-63, wherein the compartment-specific protein is further linked to a fluorescent protein. 65. The method of any one of embodiments 30-64, wherein the actuator moiety is further linked to a fluorescent protein. 66. The method of any one of embodiments 30-65, wherein the assembling occurs only in the presence of a ligand. 67. The method of embodiment 66, wherein the first dimerization domain and the second dimerization domain each bind to the ligand in the presence of the ligand. 68. The method of embodiment 66 or 67, wherein the ligand is a chemical inducer. 69. A method of controlling the spatial and temporal positioning of a compartment of a cell to a target polynucleotide, the method comprising: (a) providing a compartment-constituent protein linked to a first dimerization domain; (b) providing an actuator moiety linked to a second dimerization domain; (c) forming a complex comprising the actuator moiety and the target polynucleotide; and (d) assembling a dimer comprising the first dimerization domain and the second dimerization domain, thereby positioning the compartment around the target polynucleotide. 70. The method of embodiment 69, wherein the target polynucleotide is not endogenous to the compartment. 71. The method of embodiment 69 or 70, wherein the positioning of the compartment comprises regulating the expression of the target polynucleotide. 72. The method of embodiment 71, wherein the regulating comprises decreasing the expression of the target polynucleotide. 73. The method of embodiment 71, wherein the regulating comprises increasing the expression of the target polynucleotide. 74. The method of any one of embodiments 69-75, wherein the positioning of the compartment further comprises regulating the expression of one or more additional polynucleotides endogenous to the compartment. 75. The method of any one of embodiments 69-74, wherein the positioning of the compartment further comprises repairing a DNA break. 76. The method of any one of embodiment 75, wherein the repairing comprises introducing exogenous DNA. 77. The method of embodiment 76, wherein the introducing comprises recombination, non-homologous end-joining, or homology-directed repair. 78. The method of any one of embodiments 69-77, wherein the positioning of the target polynucleotide further comprises creating an artificial aggregate, wherein the artificial aggregate comprises protein, RNA, DNA, or a combination thereof 79. The method of any one of embodiments 69-78, wherein the target polynucleotide comprises genomic DNA. 80. The method of any one of embodiments 69-78, wherein the target polynucleotide comprises RNA. 81. The method of any one of embodiments 69-80, wherein the actuator moiety comprises a Cas protein, and wherein the system further comprises: (c) a guide RNA that complexes with the actuator moiety and hybridizes to the target polynucleotide. 82. The method of any one of embodiments 69-80, wherein the actuator moiety comprises an RNA binding protein complexed with a guide RNA that hybridizes to the target polynucleotide, and wherein the system further comprises: (c) a Cas protein that complexes with the guide RNA. 83. The method of embodiment 81 or 82, wherein the Cas protein substantially lacks DNA cleavage activity. 84. The method of any one of embodiments 81-83, wherein the Cas protein is a Cas9 protein, a Cas12 protein, a Cas13 protein, a CasX protein, or a CasY protein. 85. The method of embodiment 84, wherein the Cas12 protein is selected from the group consisting of Cas12a, Cas12b, Cas12c, Cas12d, and Cas12e. 86. The method of any one of embodiments 69-81, wherein the actuator moiety comprises a binding protein that hybridizes to the target polynucleotide, wherein the binding protein is a zinc finger nuclease or a TALE nuclease. 87. The method of any one of embodiments 69-81, wherein the actuator moiety comprises an Argonaute protein complexed with a guide polynucleotide, wherein the guide polynucleotide is a guide RNA or a guide DNA, and wherein the guide polynucleotide hybridizes to the target polynucleotide. 88. The method of any one of embodiments 69-87, wherein the compartment comprises a Cajal body. 89. The method of embodiment 88, wherein the compartment-constituent protein comprises coilin, SMN, Gemin 3, SmD1, SmE, or a combination thereof 90. The method of any one of embodiments 69-87, wherein the compartment comprises a nuclear speckle. 91. The method of embodiment 90, wherein the compartment-constituent protein comprises SC35. 92. The method of any one of embodiments 69-87, wherein the compartment comprises a PML body. 93. The method of embodiment 93, wherein the compartment-constituent protein comprises PML, SP100, or a combination thereof 94. The method of any one of embodiment 69-87, wherein the compartment is a cytosolic compartment. 95. The method of any one of embodiments 69-87, wherein the compartment is a synthetic cellular phase. 96. The method of embodiment 95, wherein the synthetic cellular phase facilitates homology directed repair. 97. The method of embodiment 96, wherein the compartment-constituent protein comprises Rad51, Rad52, RPA, MRN complex, MRX complex, CO, Sae2, BLM, Sgs1, BRCA2, exonucleases such as Exo1 or OsExo1, ATM, BRCA2, RAD54, or MDC1. 98. The method of any one of embodiments 69-97, wherein the compartment-constituent protein is further linked to a fluorescent protein. 99. The method of any one of embodiments 69-98, wherein the actuator moiety is further linked to a fluorescent protein. 100. The method of any one of embodiments 69-99, wherein the assembling occurs only in the presence of a ligand. 101. The method of embodiment 100, wherein the first dimerization domain and the second dimerization domain each bind to the ligand in the presence of the ligand. 102. The method of embodiment 100 or 101, wherein the ligand is a chemical inducer.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure.

### Example 1. Development of a Chemical-Inducible CRISPR-GO Platform for Target-Specific Genomic Repositioning

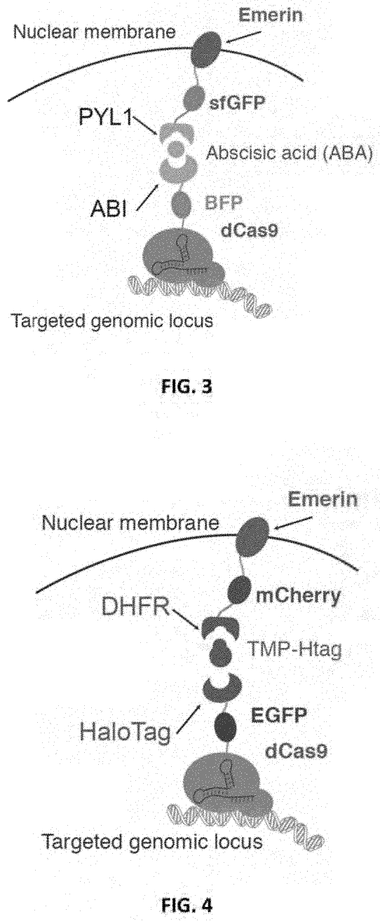

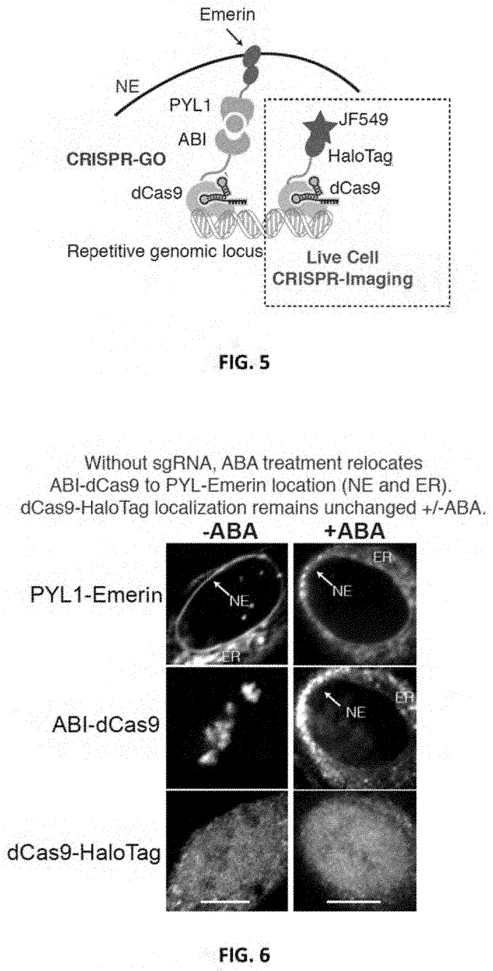

To implement an inducible CRISPR-mediated chromatin repositioning system, two chemical-inducible heterodimerization systems were tested. The first was an abscisic acid (ABA) inducible ABI/PYL1 system, and the second was a TMP-Htag (Trimethoprim-Haloligand) inducible DHFR/HaloTag system. For both systems, the Streptococcus pyogenes dCas9 (D10A & H840A) protein was fused to one heterodimer, and an inner nuclear envelope (NE) protein, Emerin, was fused to the cognate heterodimer (FIGS. 2-4). Emerin, encoded by the EMD gene, is among a group of LEM (LAP2, Emerin, MAN1)-domain proteins that mediate chromatin organization at the nuclear inner membrane. Emerin is synthesized in the cytoplasm, inserted into endoplasmic reticulum (ER), and then translocated to NE through diffusion within the contiguous ER/NE membranes (Berk et al., 2013). U2OS human bone osteosarcoma epithelial cell lines were created using lentiviral transduction that stably expressed each dimerization system. In these cell lines, addition of ABA caused spatial re-localization of ABI-BFP-dCas9 protein from within the nuclear interior to the NE and ER, due to its dimerization with PYL1-GFP-Emerin (FIGS. 5 and 6). In contrast, the TMP-Htag inducible system showed no evident effects on the co-localization of dCas9-EGFP-HaloTag and DHFR-mCherry-Emerin with the ligand. Therefore, the ABA-inducible ABI/PYL1 heterodimerization system was used for later experiments.

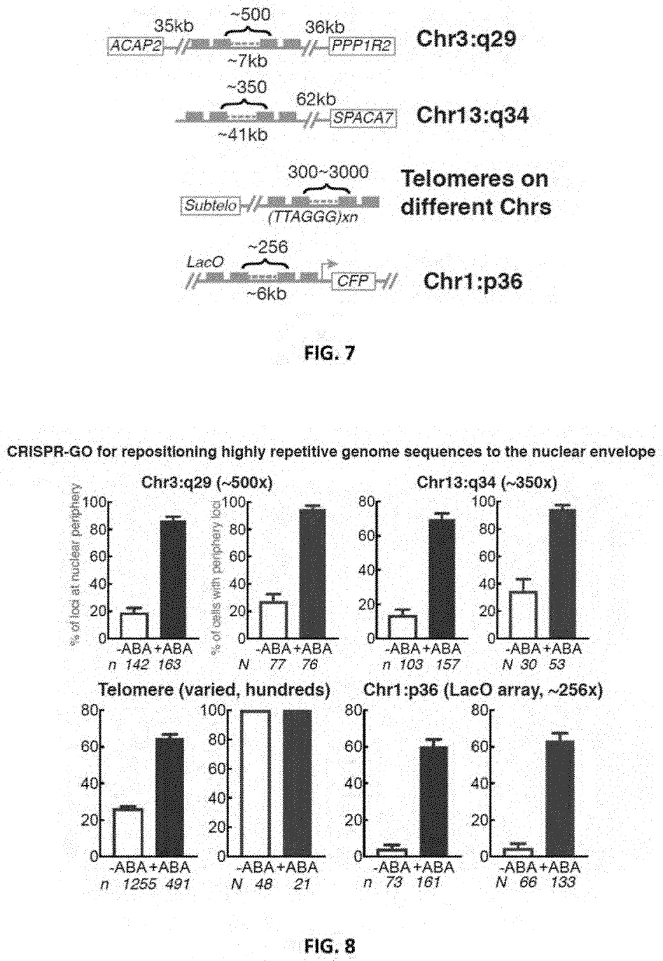

To test if the ABA-inducible CRISPR-GO system was able to alter the position of chromosomes, an endogenous locus on Chromosome 3 (Chr3) was targeted. An sgRNA targeting a highly repetitive (˜500×) region within Chromosome 3 (3q29) was lentivirally transduced into the U2OS cell line that stably expresses ABI-BFP-dCas9 and PYL1-GFP-Emerin (FIGS. 2 and 7). Given that AB1-BFP-dCas9 was mostly recruited to PYL-GFP-Emerin localization (NE and ER) after ABA treatment, another independent CRISPR-Cas9 imaging component, a dCas9-HaloTag fusion protein, was added to visualize the position of the targeted Chr3 genomic locus (FIGS. 5 and 6). In the presence of the sgChr3, the JF549-HaloTag dye was added to the culture medium to bind to dCas9-HaloTag and enable visualization of the targeted Chr3:q29 locus in living cells. The sgChr3 mediates both CRISPR-Cas9 imaging (via dCas9-HaloTag) and CRISPR-GO genomic re-localization (via AB1-dCas9) by targeting multiple repeats within the same Chr3:q29 genomic region. It was also confirmed that, in the absence of sgRNA, the dCas9-HaloTag localization was unaffected by the ABA-mediated heterodimerization between AB1-BFP-dCas9 and PYL-GFP-Emerin (FIG. 6).

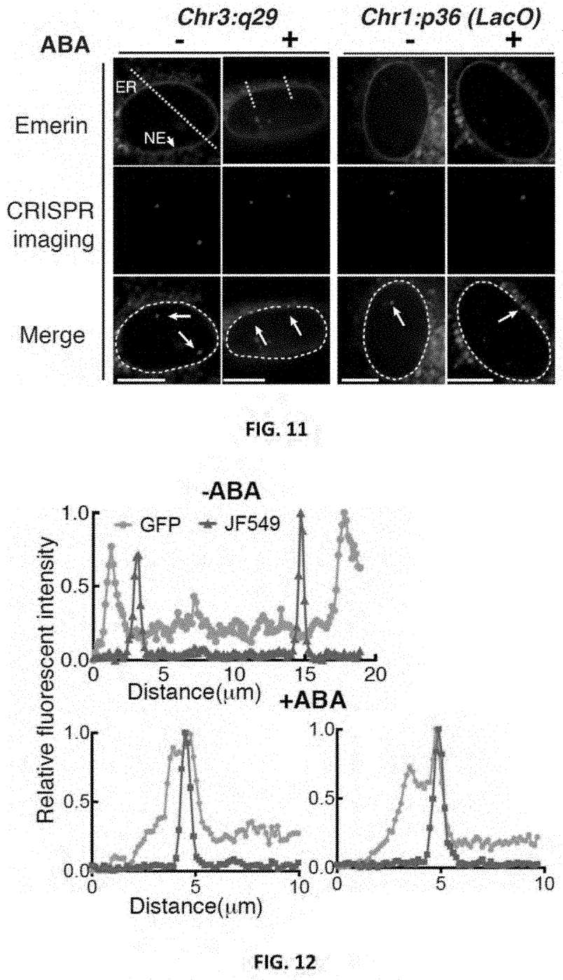

After 2 days of ABA treatment, significantly increased tethering of targeted Chr3 locus to the nuclear periphery was observed as compared to cells without ABA treatment (FIGS. 8 and 10-12). Without ABA treatment, 19% of dCas9-HaloTag labeled Chr3 loci were positioned at the nuclear periphery marked by PYL1-GFP-Emerin, while the majority of labeled loci remained within the nuclear interior (142 loci analyzed, FIG. 8). In contrast, 87% of labeled Chr3 loci repositioned to the nuclear periphery in ABA-treated cells (163 loci, FIG. 8). ABA treatment also increased the percentage of cells showing at least one Chr3 locus localized to the nuclear membrane from 27% (77 cells) to 95% (76 cells, FIG. 8). The significant increase of both repositioned genomic loci (p<0.0001) and cells (p<0.0001) with chemical treatment suggests that the systems disclosed herein are efficient in repositioning highly repetitive polynucleotides such as endogenous genomic loci in cells.

### Example 2. Efficient Repositioning of Repetitive Genomic Loci by the CRISPR-GO System

In addition to the Chr3:q29 locus, repositioning other highly repetitive endogenous genomic loci, including Chr13 locus and telomeres, to the nuclear periphery was tested. Using an sgRNA targeting repetitive region (˜350× repeats) on Chromosome 13q34 (Chr13) (FIG. 7), the percentage of tethered Chr13 loci increased from 13% (n=103) to 69% (n=157, p<0.0001), and the percentage of cells containing at least one periphery-localized locus increased from 34% (n=30) to 94% (n=53, FIG. 8, p<0.0001). Similarly, CRISPR-GO-containing cells with a telomere-targeting sgRNA were transduced to test whether telomeres could also be repositioned with our system. TRF1-mCherry, a telomere marker, was also co-expressed to visualize telomeres. In this case, the percentage of periphery-localized telomere loci increased from 26% (n=1255) to 65% (n=491, FIG. 8, p<0.0001).

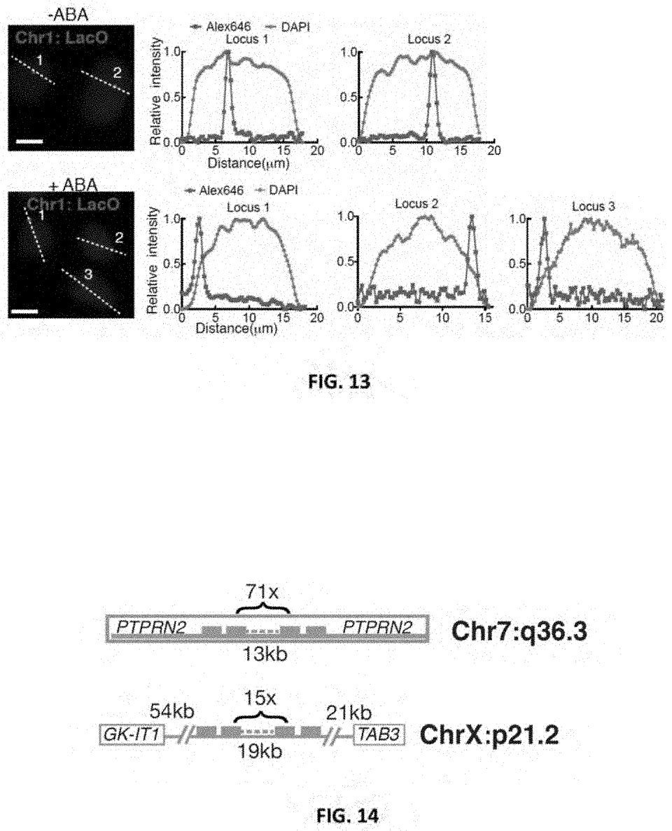

A synthetically integrated LacO array located at Chromosome 1p36 was also targeted in a U2OS 2-6-3 reporter cell line previously used for studying chromosome repositioning (FIG. 7). Using an sgRNA targeting the LacO sequence, CRISPR-Cas9 imaging in living cells revealed that the percentage of nuclear periphery-tethered targeted genomic loci increased from 4% (n=73) to 60% (n=161, p<0.0001), and the percentage of cells containing at least one periphery-localized locus increased from 4.5% (n=66) to 65% (n=133) (FIG. 8, 1G, p<0.0001). Fluorescent in situ hybridization (FISH) staining in fixed cells with DAPI further confirmed that the majority of LacO loci localized at the nuclear periphery after ABA treatment (FIG. 13).

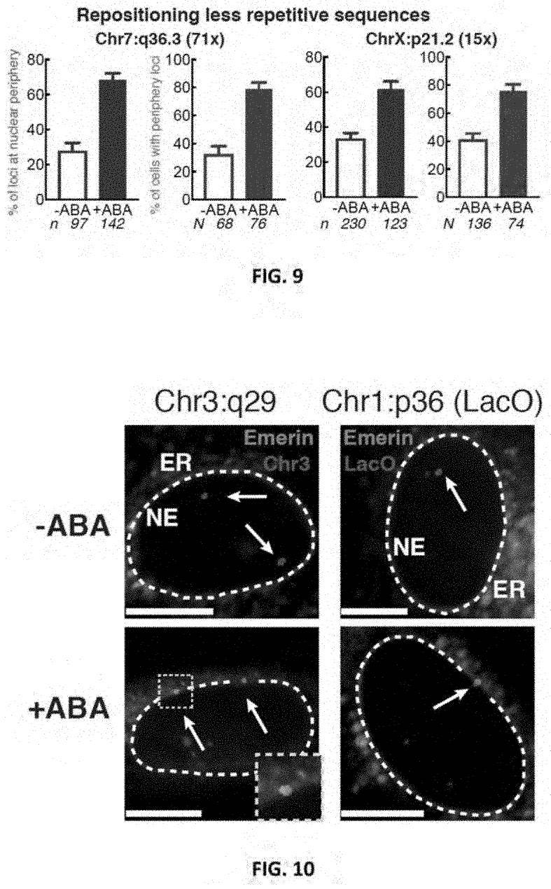

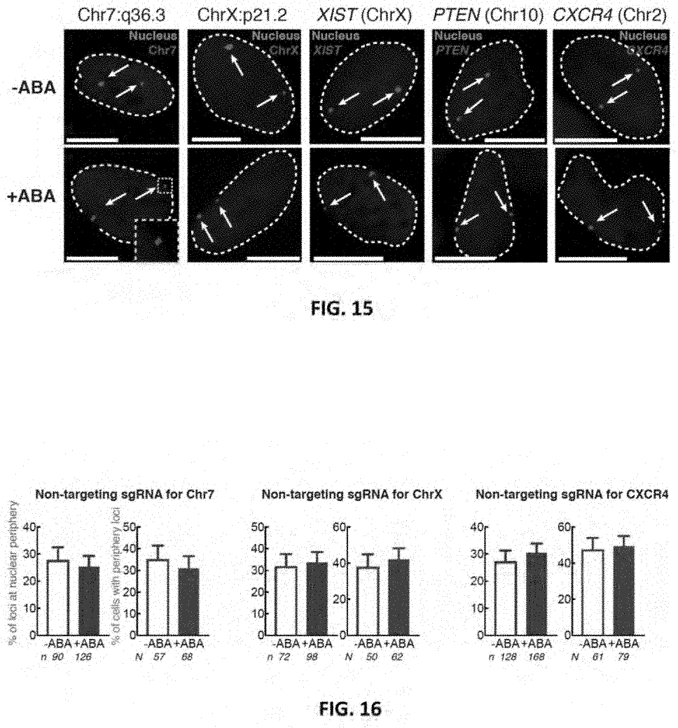

The efficiency of the CRISPR-GO system in repositioning less repetitive (<100 repeats) sequences was next tested. A genomic region on Chr7 q36.3 containing ˜71 sgRNA-targetable repeats and a genomic region on ChrX p21.2 containing ˜15 sgRNA-targetable repeats were chosen as targets (FIG. 14). We visualized the position of the targeted genomic loci was visualized using 3D-FISH and the nucleus was stained by DAPI (FIG. 15). After 3 days of ABA treatment, the percentage of periphery-localized loci increased from 28% (n=97) to 68% (n=142, p<0.0001) for Chr7 and from 33% (n=230) to 62% (n=123, p<0.0001) for ChrX. The percentage of cells containing at least one periphery-adjacent locus increased from 32% (n=68) to 79% (n=76, p<0.0001) for Chr7 and from 41% (n=136) to 76% (n=74, p<0.0001) for ChrX (FIG. 9). When using a non-targeting sgRNA as a control, the percentages of Chr7 and ChrX at the nuclear periphery were similar to those seen with non-ABA treated samples and remained unchanged after ABA treatment (FIG. 16). Together, these results suggest that the chemical inducible systems provided herein are efficient in repositioning highly repetitive and less repetitive sequences to cellular compartments such as the nuclear periphery.

### Example 3. Efficient Repositioning of Non-Repetitive Genomic Loci by the CRISPR-GO System

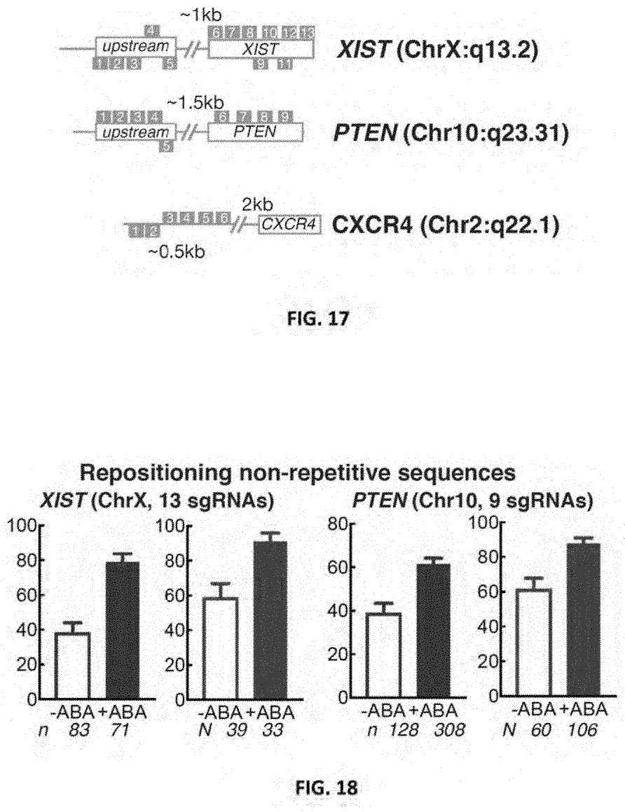

Though repetitive sequences are abundantly present in human genome, it is of further interest if the CRISPR-GO system enabled repositioning non-repetitive genomic loci. The non-repetitive gene XIST located at ChrX q13.2, was first targeted and 13 sgRNAs tiling the XIST genomic region were designed (FIG. 17). All constructs were lentivirally transduced into U2OS cells that stably expressed the CRISPR-GO system. With ABA treatment, the percentage of periphery-localized XIST loci increased from 39% (n=83) to 79% (n=71, p<0.0001), and the percentage of cells containing periphery-localized loci increased from 59% (n=39) to 90% (n=33) (FIG. 18, 1H, p=0.0028). Using a pool of 9 sgRNAs targeting regions adjacent to and within the gene PTEN at Chr10 (FIG. 17), the CRISPR-GO system increased the percentage of periphery localized PTEN loci from 39% (n=128) to 61% (n=308, p<0.0001) and the percentage of cells containing at least one periphery-adjacent locus increased from 62% (n=60) to 88% (n=106, p=0.0002) (FIGS. 15 and 18).

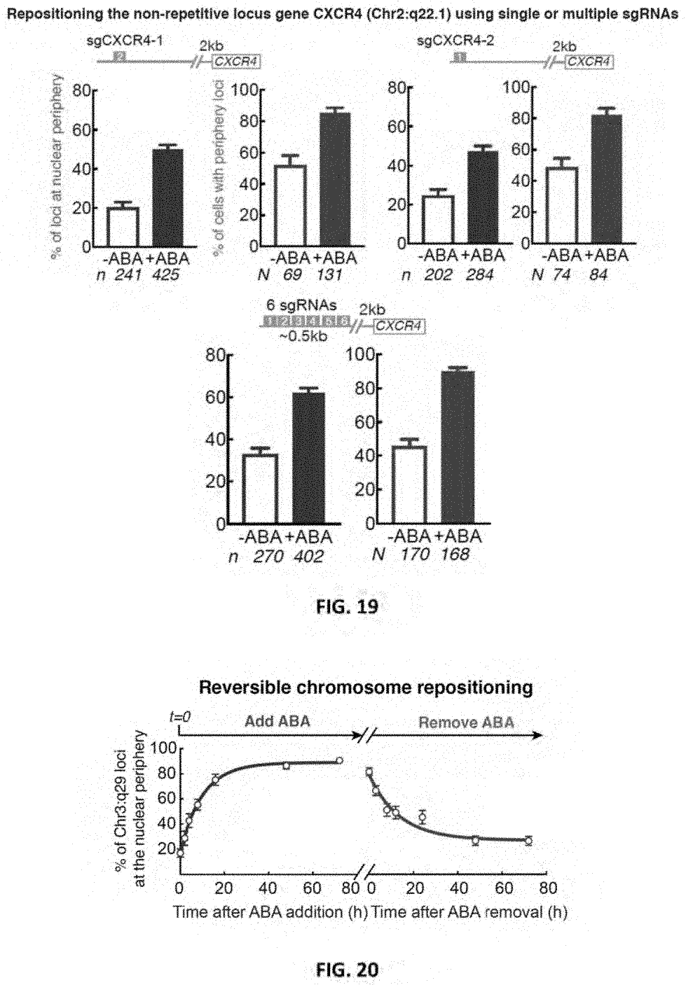

Whether a single sgRNA targeting a non-repetitive region is sufficient to re-reposition a genomic locus was also tested. Using a single sgRNA (sgCXCR4-1) targeting the CXCR4 locus at Chr2, the percentage of periphery localized CXCR4 loci increased from 20% (n=241) to 50% (n=425, p<0.0001), and the percentage of cells containing periphery-localized loci increased from 52% (n=69) to 85% (n=131, p<0.0001) (FIG. 19). Similarly, another single sgRNA 25 (sgCXCR4-2) increased localized CXCR4 loci from 25% (n=202) to 47% (n=284), and cells from 49% (n=74) to 82% (n=84, p<0.0001) (FIG. 19). Efficiency when targeting the CXCR4 locus using single sgRNA and a pool of 6 sgRNAs was compared. When using multiple sgRNAs, the CRISPR-GO system increased the percentage of localized CXCR4 loci from 32% (n=270) to 62% (n=402, p<0.0001) and the percentage of cells from 46% (n=170) to 90% (n=168, p<0.0001), respectively (FIGS. 15 and 19). When using a non-targeting sgRNA as a control, the percentage of CXCR4 loci at the nuclear periphery was similar to that seen with non-ABA treated samples and remained unchanged after ABA treatment (FIG. 16). These results together confirmed that the systems disclosed herein can mediate efficient re-localization of non-repetitive loci to the cellular compartments such as the nuclear periphery.

### Example 4. Chemically Inducible and Reversible CRISPR-GO-Mediated Genomic Repositioning

One advantage of the provided systems and methods is the ability to easily switch on or off polynucleotide re-positioning by adding or removing a chemical inducer to the culture medium. Chemical induction and removal experiments were performed to study the dynamics and reversibility of the ABA-inducible CRISPR-GO system (FIG. 20). To study chemical induction, U2OS cells containing the CRISPR-GO system targeting Chr3 loci were treated with ABA and examined at different time points. For chemical reversal, U2OS cells containing the CRISPR-GO system targeting Chr3 loci were first treated with ABA for 2 days, and then switched to medium without ABA. ABA-induced genomic re-localization occurred relatively quickly as the percentage of Chr3 loci tethered to nuclear membrane increased from 19% (n=142) to 75% (n=93, p<0.0001) within 16 hours of ABA addition, and reached 91% (n=160) after 72 hours of ABA addition. After ABA removal, the percentage Chr3 loci tethered to nuclear membrane decreased from 82% (n=163) to 45% (n=84, p<0.0001) after 24 hours, and further decreased to 27% (n=146) and 26% (n=159) after 48 hours and 72 hours, respectively. After 48 hours and 72 hours of ABA removal, the percentage of Chr3 at the nuclear periphery was indistinguishable from a control sample with no ABA treatment (25%, n=106, p=0.77) imaged at the same time, suggesting that ABA removal fully reversed the genomic repositioning effects mediated by the CRISPR-GO system within 48 hours (FIG. 20). These results suggest that nuclear repositioning mediated by the systems and methods disclosed herein can be easily switched on and off by, for example, adding or removing a chemical inducer such as ABA.

### Example 5. Mitosis-Dependent and Mitosis-Independent Mechanisms for CRISPR-GO System-Mediated Nuclear Periphery Repositioning

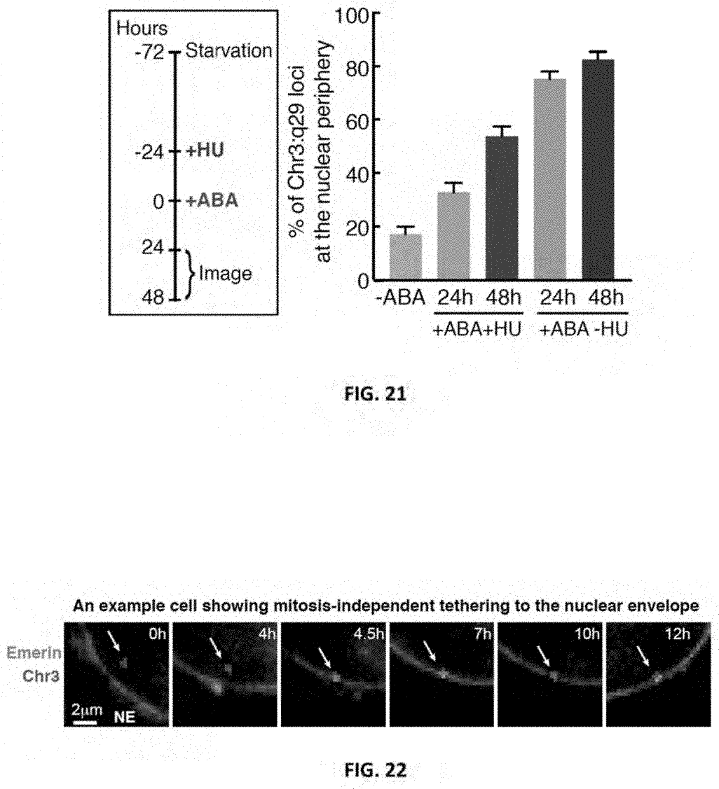

The CRISPR-GO system was used to target the endogenous Chr3 locus. CRISPR-GO cells containing Chr3-targeting sgRNAs were synchronized and arrested in the S phase by serum starvation and Hydroxyurea (HU) treatment and then treated with ABA for chemical induction (FIG. 21). Interestingly, after 24 hours of ABA treatment, the percentage of nuclear periphery tethered Chr3 loci increased from 17% (n=175) to 33% (n=177, p=0008) in HU treated S-phase arrested cells, which was significantly lower than the percentage in unsynchronized cells (75%, n=251, p=0.0001). After 48 hours of ABA treatment, the percentage of nuclear periphery tethered Chr3 loci increased to 54% (n=177, p<0.0001) in HU treated S-phase arrested cells, which is also lower than in unsynchronized cells (83%, n=178, p<0.0001). Thus, in HU treated S-phase arrested cells, nuclear periphery repositioning was still observed, but to a less extent compared to cells that underwent mitosis. These results suggest that repositioning of endogenous loci to the cellular compartments such as the nuclear periphery using the systems and methods disclosed herein may happen via both mitosis-dependent and mitosis-independent mechanisms.

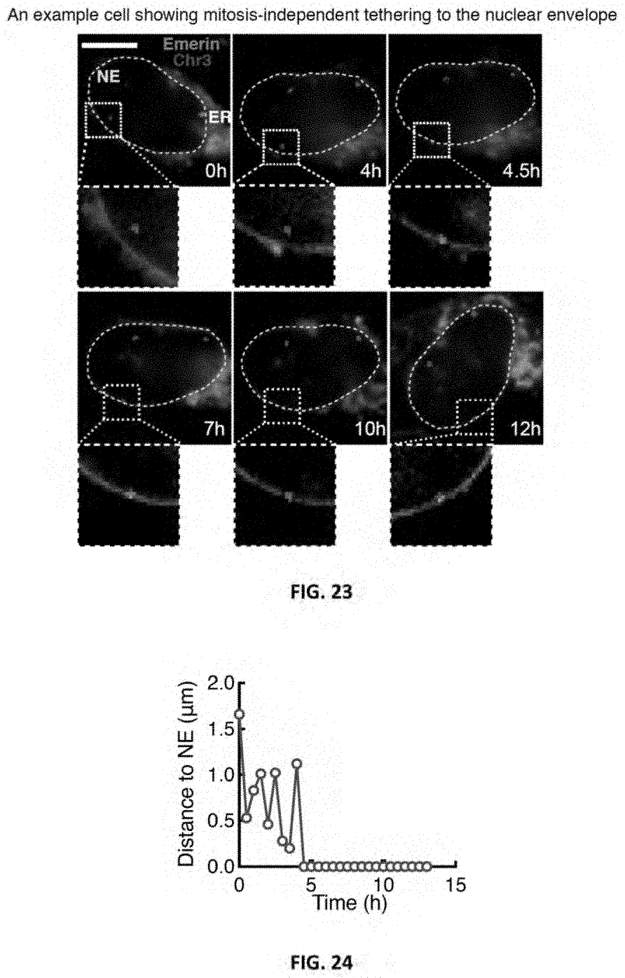

Mitosis-independent periphery repositioning has not yet been reported to our knowledge. To probe whether a genomic locus can be tethered to the nuclear periphery during interphase, live-cell CRISPR-Cas9 imaging was used to track the dynamics of nuclear periphery tethering. The dynamic process of endogenous Chr3 loci becoming tethered to nuclear periphery during interphase was detected. In the representative example shown in FIGS. 22 and 23, a Chr3:q29 locus (arrow) started off separate from the nuclear periphery (GFP-Emerin) during the first 4 hours of recording, became tethered to the nuclear periphery at 4.5 hours and then stayed tethered for the remaining 8 hours of recording, even while the nucleus underwent a rotation between 10 hours and 12 hours. We quantified the distance between this Chr3 locus and the nearest nuclear periphery over time, and found that the distance stayed as 0 after tethering occurred at 4.5 hours (FIG. 24). These results suggest that, despite the relatively stable organization of chromatin structure during the interphase, a genomic locus located close to the nuclear periphery is able to be synthetically tethered to the nuclear periphery in a mitosis-independent manner using the systems and methods disclosed herein.

### Example 6. Suppression of Movements of Endogenous Genomic Loci by Tethering at the Nuclear Periphery

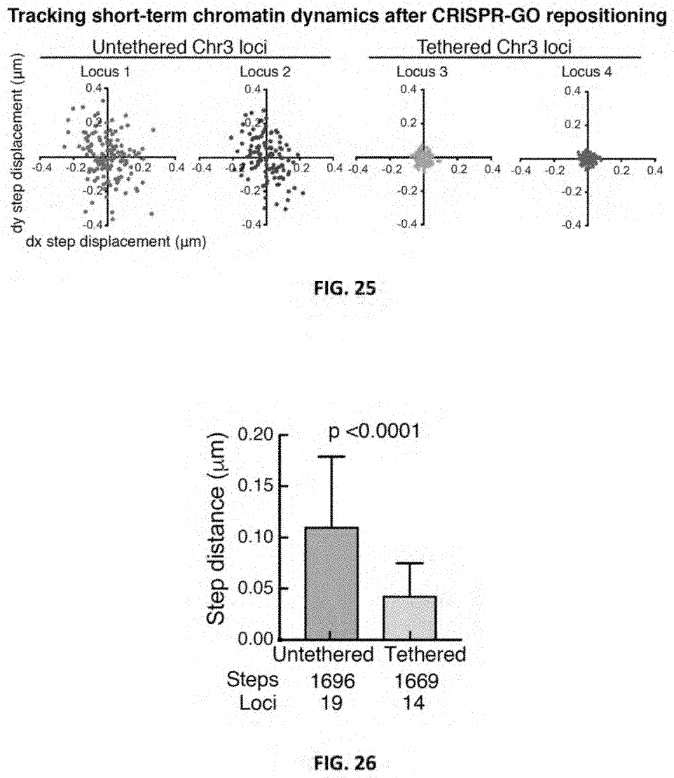

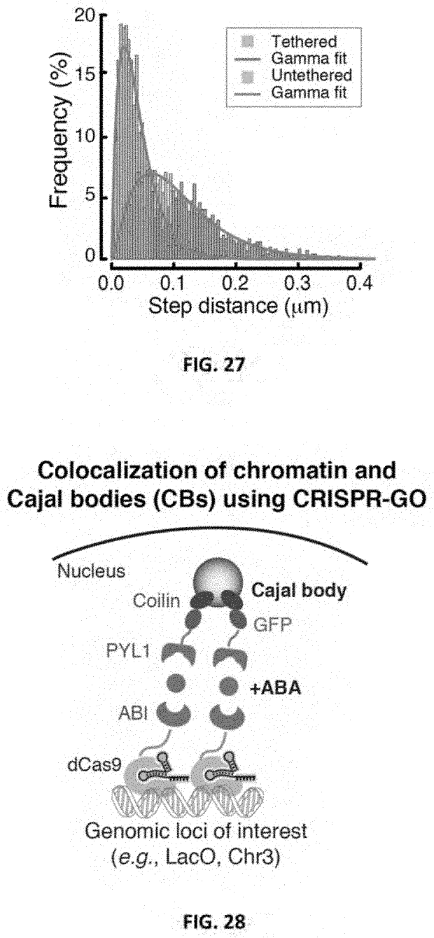

The short-time movement kinetics of genomic loci after genomic tethering was studied by combining the CRISPR-GO system with CRISPR-Cas9 imaging in living cells. The short-term dynamics of Chr3 loci tethered at the nuclear periphery were examined as compared to 30 untethered loci (FIG. 25). Images were taken every 4-6 s under a confocal microscope. We observed that the untethered Chr3 loci were more mobile than the tethered Chr3 loci (FIG. 25). We characterized the displacement between the consecutive movement steps of each locus in a 2-dimensional space (dx^{t}=x^{t}−x^{t-1} & dy^{t}=y^{t}−y^{t-1}, where (x^{t},y^{t}), is the coordinate of a locus at time t), and plotted their distribution as a measure of movement amplitude (FIG. 25). The untethered Chr3 loci displayed a broader distribution of step displacement with higher amplitude compared to the periphery-tethered loci. The observation that the tethered genomic loci exhibited more confined movement confirms the physical tethering of these loci to the nuclear envelope. We further quantified the average Euclidean step distance (√(x^{t}−x^{t-1})²+(y^{t}−y^{t-1})²). We found that untethered Chr3 loci showed a step distance of 0.11±0.07 μm (1696 steps for 19 loci), and the periphery-tethered Chr3 loci presented a much lower step distance of 0.04±0.03 μm (1669 steps for 14 loci) (p<0.0001, FIG. 26). The step distances of the untethered and tethered loci can both be well approximated by distinct gamma distributions (FIG. 27). These results suggest that cellular compartment tethering mediated by the systems and methods disclosed herein can suppresses the mobility and dynamics of targeted polynucleotides.

### Example 7. Colocalization of Genomic Loci with Membraneless Nuclear Bodies Including Cajal Bodies and PML Bodies

Whether the CRISPR-GO system can mediate colocalization of chromatin loci with membraneless nuclear bodies was next tested. Genomic loci were chosen to recruit to Cajal 20 bodies (CBs). To do this, a Cajal body-targeting CRISPR-GO system was designed by fusing PYL1 with Coilin, a marker of Cajal bodies. PYL1-GFP-Coilin and ABI-dCas9 were introduced into U2OS cells via lentiviral transduction (FIG. 28). We tested the recruitment efficiency in the U2OS 2-6-3 cells containing a LacO repeat array inserted in Chr1:p36.

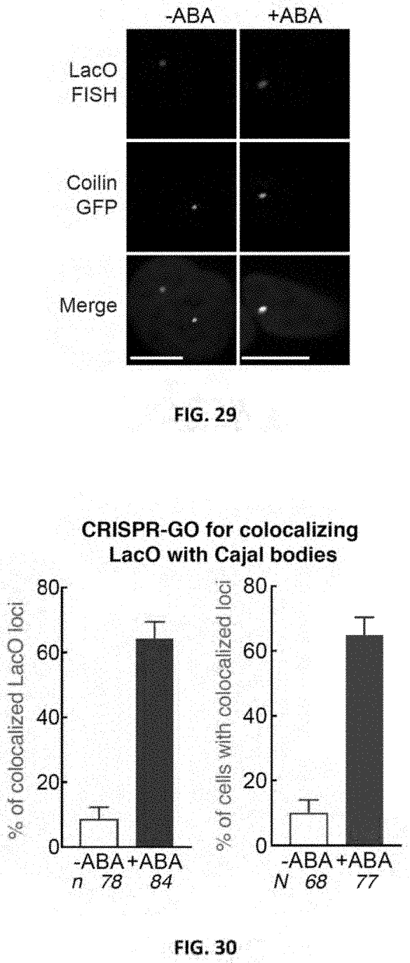

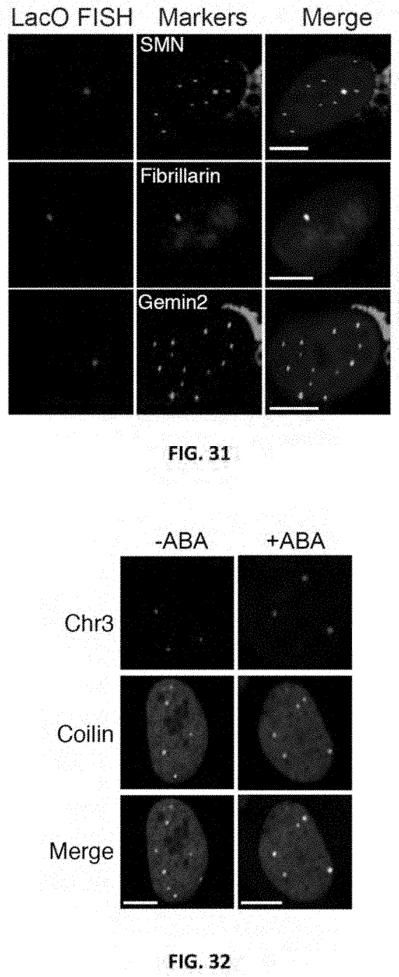

Using an sgRNA targeting the LacO sequence, we visualized the spatial positioning of the LacO array using 3D-FISH and the location of CBs by GFP-Coilin after 2 days of ABA treatment (FIG. 29). The percentage of LacO loci that colocalized with GFP-Coilin-labeled CBs increased from 9% (n=78) without ABA treatment to 64% (n=84) after ABA treatment, and the percentage of cells containing at least one CB colocalized with a LacO locus increased from 10% (n=68) to 65% (n=77) (FIG. 30, p<0.0001). Combined immunostaining with FISH showed that other CB components (SMN, Fibrillarin, and Gemin2) also colocalized with LacO loci after 20 hours of ABA treatment (FIG. 31), confirming CRISPR-GO-mediated colocalization of the targeted genomic loci with CBs.

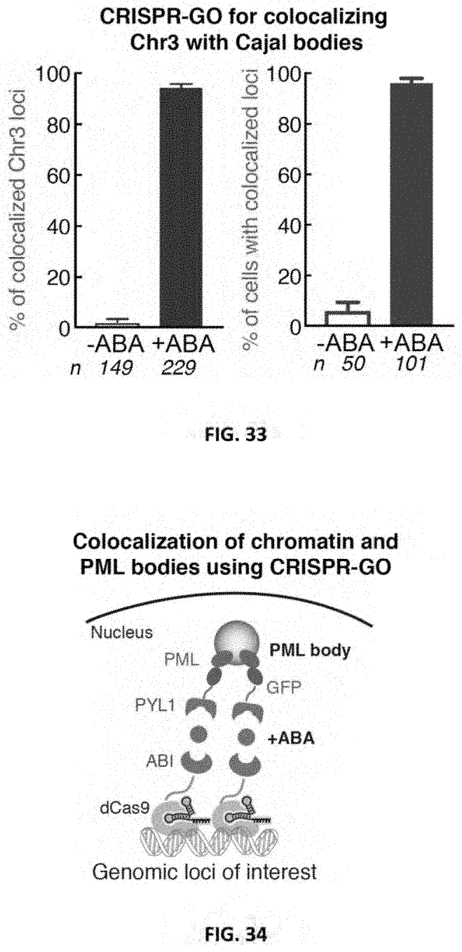

To target endogenous genomic loci to CBs, the Chr3:q29-targeting sgRNA was introduced into U2OS cells expressing the Cajal body-targeting CRISPR-GO system. Significant colocalization was observed between the Chr3 loci (visualized with CRISPR-Cas9 imaging) and CBs (visualized with GFP-Coilin) 24 hours after ABA treatment (FIG. 32). The percentage of Chr3 loci that colocalized with CBs increased from 2% (n=149) before ABA treatment to 94% (n=229, p<0.0001) after ABA treatment, and the percentage of cells containing at least one CB colocalized with a Chr3 locus increased from 6% (n=50) to 96% (n=101, p<0.0001) (FIG. 33).

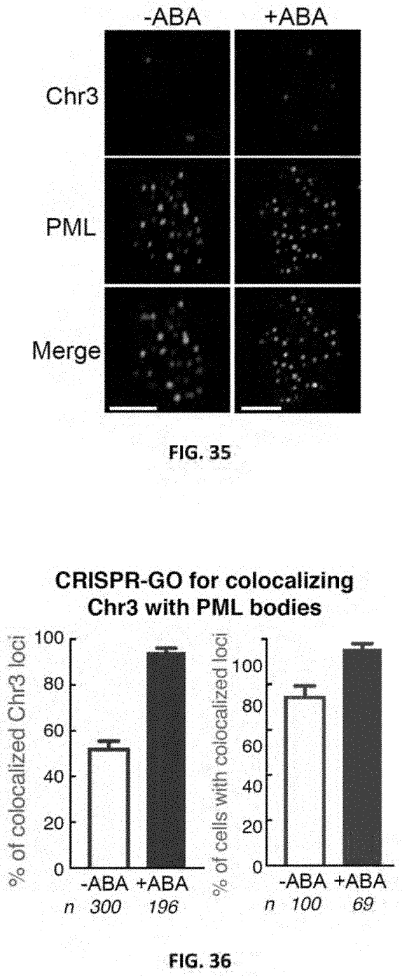

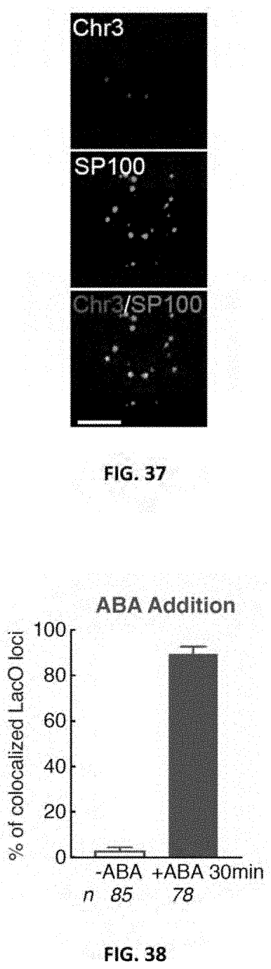

Whether CRISPR-GO could mediate colocalization of chromatin loci with PML nuclear bodies was also tested. To do this, a PML body-targeting CRISPR-GO system was designed by fusing PYL1 with the PML gene, the scaffold protein of PML bodies. To target endogenous genomic loci to PML bodies, the Chr3:q29-targeting sgRNA was introduced into cells expressing both PYL1-GFP-PML and ABI-dCas9, the positioning of Chr3 loci was visualized by CRISPR-Cas9 imaging and the position of PML bodies was visualized by GFP-PML (FIGS. 34 and 35). Interestingly, a high percentage (52.6%, n=300) of Chr3:q29 loci colocalized with the PML bodies without ABA treatment, which may suggest natural Chr3:q29-PML body colocalization (FIGS. 35 and 36). After ABA treatment, the percentage of target Chr3 loci that colocalized with PML bodies increased to 94% (n=196, p<0.0001), and the percentage of cells containing at least one PML body colocalized with a Chr3 locus increased from 75% (n=100) to 96% (n=69, p=0.0003) (FIGS. 35 and 36). Immunostaining also confirmed that Chr3 loci colocalized with SP100, another PML body marker (FIG. 37).

### Example 8. Rapid, Inducible, and Reversible CRISPR-GO Mediated Association Between Target Genomic Loci and Cajal Bodies

Chemical induction and removal experiments were performed to study the dynamics and reversibility of the CRISPR-GO mediated chromatin colocalization with CBs. Using the LacO locus inserted at Chr1:p36 in U2OS 2-6-3 cells as an example, we observed that the association between LacO loci and GFP-Coilin-marked CBs occurred rapidly: within 30 minutes after ABA addition, the percentage of LacO loci that colocalized with CBs increased from 2.6% (n=78) to 89% (n=85, p<0.0001) (FIG. 38).

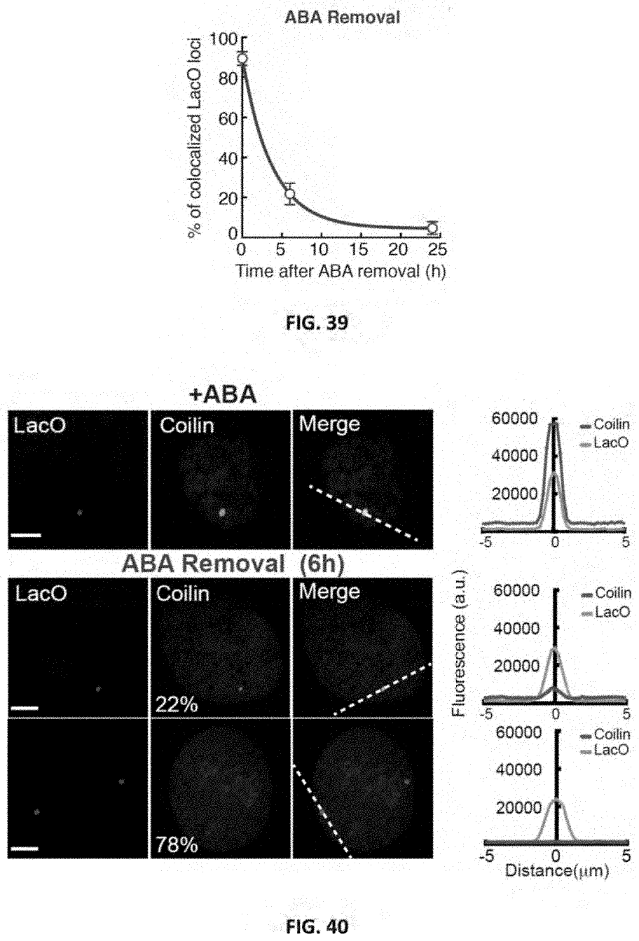

In cells pretreated with ABA for 1 day, ABA removal was observed to lead to dissociation of CBs from LacO loci. After ABA removal, the percentage of the targeted LacO loci that colocalized with CBs decreased from 89% (n=85) to 22% (n=60, p<0.0001) after 6 hours and further decreased to 4.6% (n=45, p<0.0001) after 24 hours (FIG. 39). At 6 hours after ABA removal, among the cell population (22%) that still possessed LacO-colocalized GFP-Coilin, the remaining GFP-Coilin intensity was much dimmer than that in cells undergoing sustained ABA treatment (FIG. 40), which may suggest a gradual disassembly process of CBs after ABA removal.

### Example 9. De Novo CB Formation and Repositioning of Existing CBs at Targeted Chromatin Loci

To further characterize the dynamics of CRISPR-GO-mediated association of Cajal bodies with targeted genomic loci, time-lapse microscopic imaging of individual cells was performed before and after ABA treatment. Theoretically, colocalization between a genomic locus and nuclear bodies could occur through de novo formation of a nuclear body at the genomic locus, or through repositioning the genomic locus to an existing nuclear body. Previous reports using the LacO-Lacl tethering system suggest that Cajal bodies form de novo at the targeted DNA site.

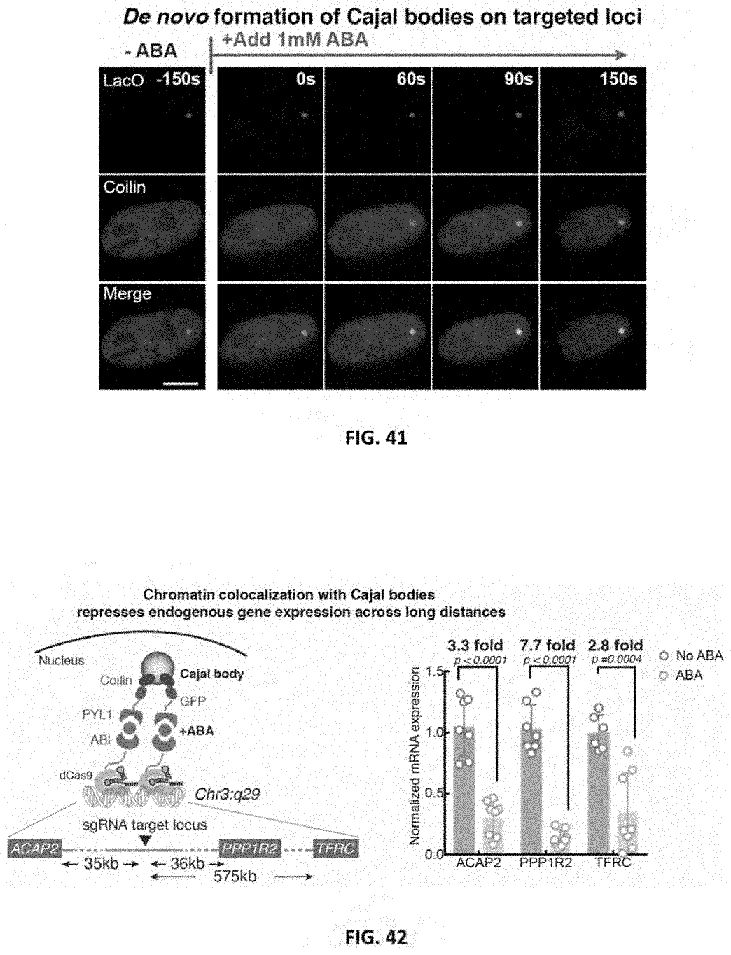

Using the CRISPR-GO system to target LacO loci to Cajal bodies, rapid (within 20 minutes) de novo CBs formation was observed at the LacO locus in most analyzed cells after addition of ABA (FIG. 41). For example, in a cell with no initial GFP-Coilin accumulation at a LacO locus without ABA (FIG. 41, −150 s), ABA treatment (added between −150 s and 0 s, FIG. 42) rapidly recruited GFP-Coilin to the LacO locus (FIG. 41, 150s), leading to de novo formation of Cajal bodies. The GFP-Coilin fluorescence intensity at the LacO loci approached saturation within 10 minutes after ABA addition (FIG. 44).

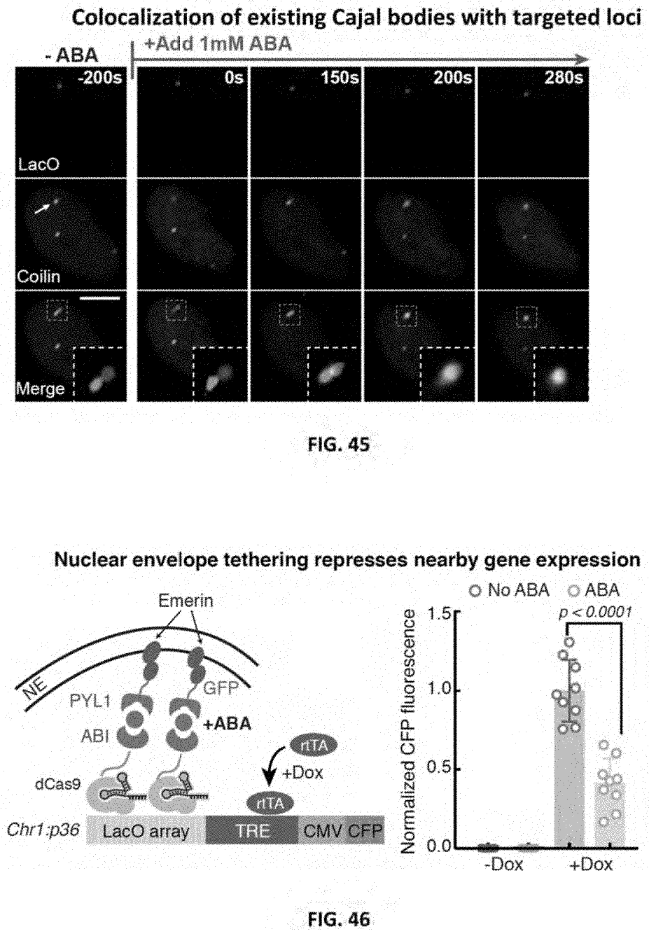

Interestingly, dynamic repositioning of the targeted chromatin locus with an existing CB was observed if the two were initially spatially close to each other. For example, in a cell where an existing CB was adjacent to a LacO locus without ABA treatment (FIG. 45, −200 s), ABA treatment (added between −200 s and Os, FIG. 45) led to rapid colocalization of the existing CB and the LacO locus, suggesting that the systems disclosed herein can also mediate direct association between polynucleotides (e.g., genomic loci) and cellular compartments (e.g., existing nuclear bodies), a phenomenon that has not yet been reported before.

### Example 10. Reduced Reporter Gene Expression Resulting from CRISPR-GO-Mediated Relocalizing of Genomic Loci to the Nuclear Periphery

Previous studies offer different evidence about the effect that genomic relocalization to the nuclear periphery has on gene expression. Some studies showed that tethering LacO repeats to the nuclear periphery using LacI-Emerin or LacI-Lap2β caused repression of adjacent gene. Other studies showed re-localization of the LacO array to the nuclear periphery using a LacI-Lamin B1 fusion protein in the U2OS 2-6-3 cells, but observed no obvious changes in adjacent gene expression. CRISPR-GO offers another way to study this question, since it is much easier to test the effects of recruiting different chromosome loci to the nuclear periphery.

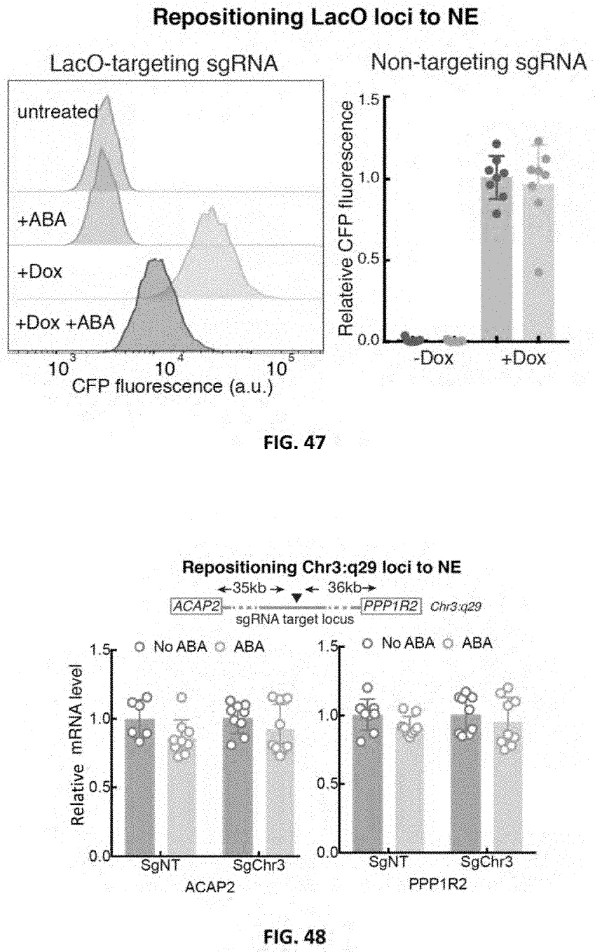

Whether CRISPR-GO-mediated repositioning of the LacO array to the nuclear periphery could influence gene expression was examined. The LacO locus in the U2OS 2-6-3 cells is located upstream of a Doxycycline (Dox)-inducible TRE (Tetracycline responsive element)-CMV promoter that drives expression of a CFP reporter (FIG. 46). The adjacent CFP reporter expression in both ABA-treated and untreated cells were measured by flow cytometry, and ABA-treated cells were observed to show consistently decreased reporter gene expression compared to untreated cells (a reduction of 59%, FIGS. 46 and 47). This gene repression effect was similar to repositioning the LacO locus to the nuclear periphery using a Lacl-Emerin fusion protein. As a control to confirm that gene repression was target-specific, we also tested a non-targeting sgRNA, and observed no decrease of the reporter gene expression (FIG. 47).

Whether repositioning endogenous genomic loci to the nuclear periphery could alter gene expression was next tested. The Chr3, XIST, and CXCR4 loci were repositioned to the nuclear periphery individually, and RT-qPCR was performed to detect changes in adjacent gene expression (Chr3: ACAP2 & PPP1R2; CXCR4; XIST). Surprisingly, no evidence of gene expression change was seen for these genes (e.g., ACAP2 & PPP1R2 in FIG. 48). Thus, it raises questions whether repositioning a gene adjacent to the nuclear periphery is sufficient to cause endogenous gene expression changes, and it remains possible that repositioning-induced gene expression changes are locus-dependent.

### Example 11. Reduced Reporter Gene Expression Resulting from CRISPR-GO-Mediated Relocalizing of Genomic Loci to CBs

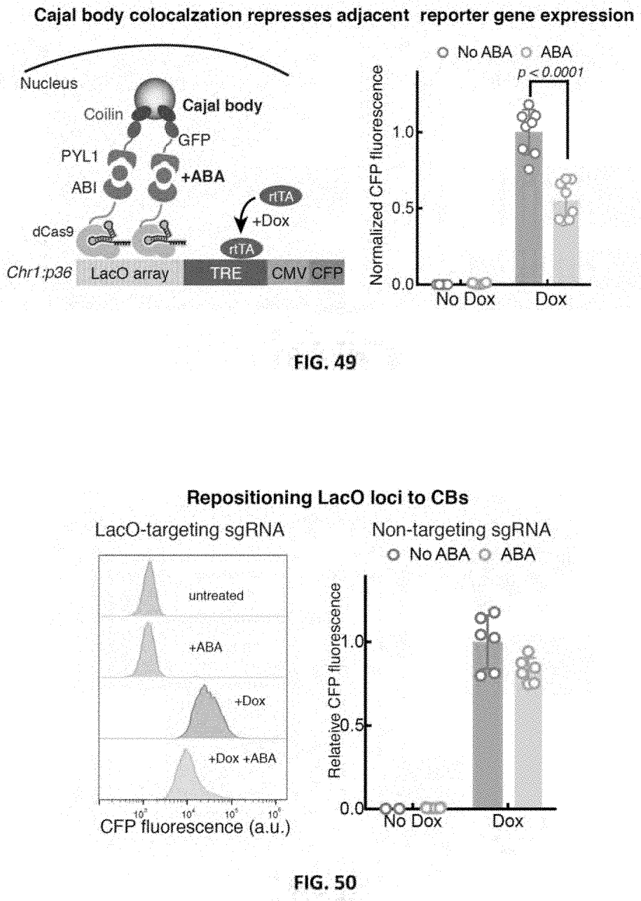

Whether colocalization of LacO loci to CBs using the CRISPR-GO system in the U2OS 2-6-3 cell line was sufficient to influence adjacent gene expression was next tested (FIG. 49). Cells were treated with ABA for 2 days, induced with Dox for 1 day, and measured the CFP expression by flow cytometry. Consistently decreased reporter gene expression was observed in ABA-treated cells compared to untreated cells (an average reduction of 45%, FIGS. 49 and 50, p<0.0001). To confirm this gene repression effect is target-specific, a non-targeting sgRNA was tested, and a slight but not significant decrease (p>0.05) of the reporter gene expression was observed (FIG. 50).

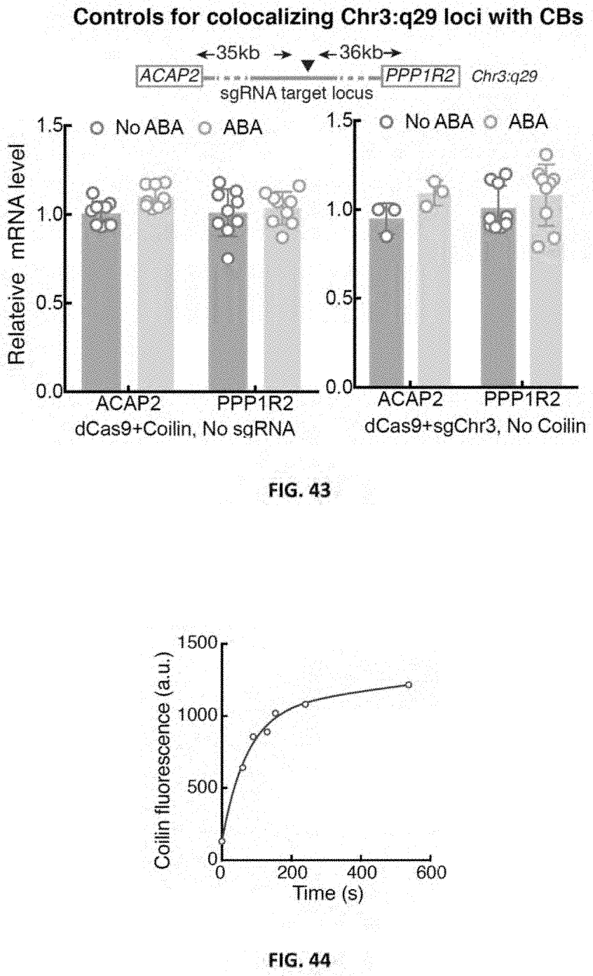

Whether colocalizing an endogenous genomic locus to CBs could alter adjacent gene expression was next tested. The CRISPR-GO system was used to induce colocalization of Chr3:q29 with CBs (FIGS. 32 and 33) and then RT-qPCR was performed to detect changes in adjacent gene expression (Chr3: ACAP2 &PPP1R2) after 4 days of ABA treatment. Surprisingly, significant repression of both adjacent genes compared to untreated cells was observed. ACAP2, located about 35 kb upstream of the CB-targeting loci on Chr3, exhibited 3.3 fold of repression after ABA treatment (p<0.0001, FIG. 42), and PPP1R2, located about 36 kb downstream of the CB-targeting loci on Chr3, exhibited 7.7 fold of repression (p<0.0001, FIG. 42). Cells without sgRNAs or without the PYL1-GFP-Coillin component were confirmed as showing no changes in ACAP2 and PPP1R2 gene expression (FIG. 43). Together, these results show that targeted colocalization of a given polynucleotide (e.g., genomic locus) with a cellular compartment (e.g., CBs) is able to repress adjacent polynucleotide expression. The long-distance efficacy of, for example, gene expression perturbation mediation using the systems and methods disclosed herein stands in contrast to CRISPRi or CRISPRa, which only cause perturbations in gene expression a relatively short distance away from the dCas9 binding site. The observation that the provided methods and systems are able to mediate long-distance polynucleotide expression perturbations may provide a useful new means of, for example, gene regulation.

### Example 12. Altered Cellular Phenotypes Resulting from CRISPR-GO-Mediated Telomere Repositioning

The CRISPR-GO system was used to investigate how telomere reorganization to nuclear compartments affected cellular phenotype. Among all genomic loci tested, the dynamics of telomeres are the best studied and are shown to be associated with the nuclear periphery and CBs at certain stages of the cell cycle. Given the important role of telomeres for genome integrity, their interactions with nuclear compartments may have functional implications. For example, during the cell cycle, telomeres are dynamically tethered to the nuclear envelope when the nuclear membrane reassembles in post-mitotic cells, and then relocate to the interior of the nucleus during the G1 phase, where they remain for the rest of cell cycle. The cycle of telomere tethering and untethering to the nuclear envelope may be important for chromatin organization and the cell cycle/viability.

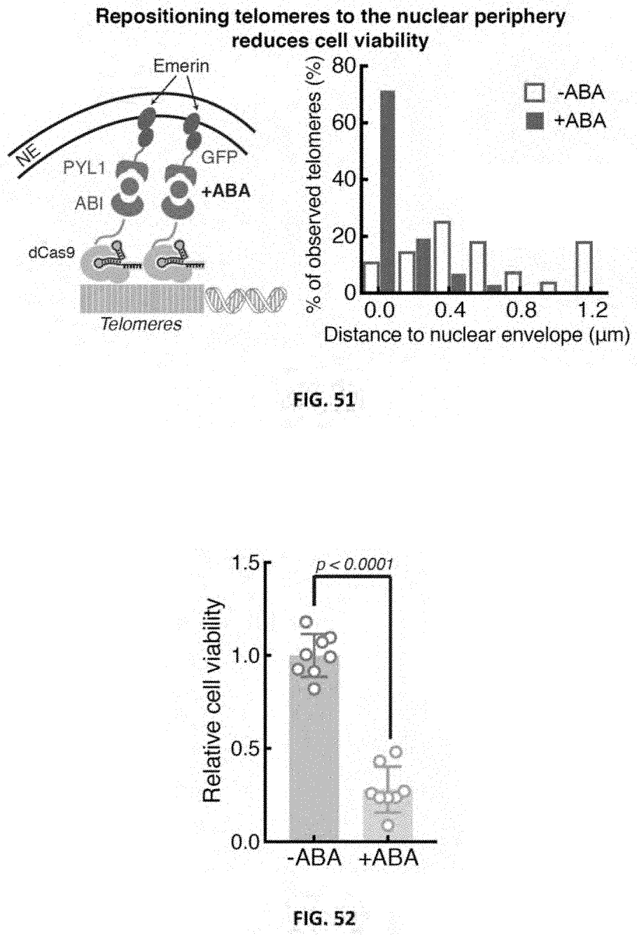

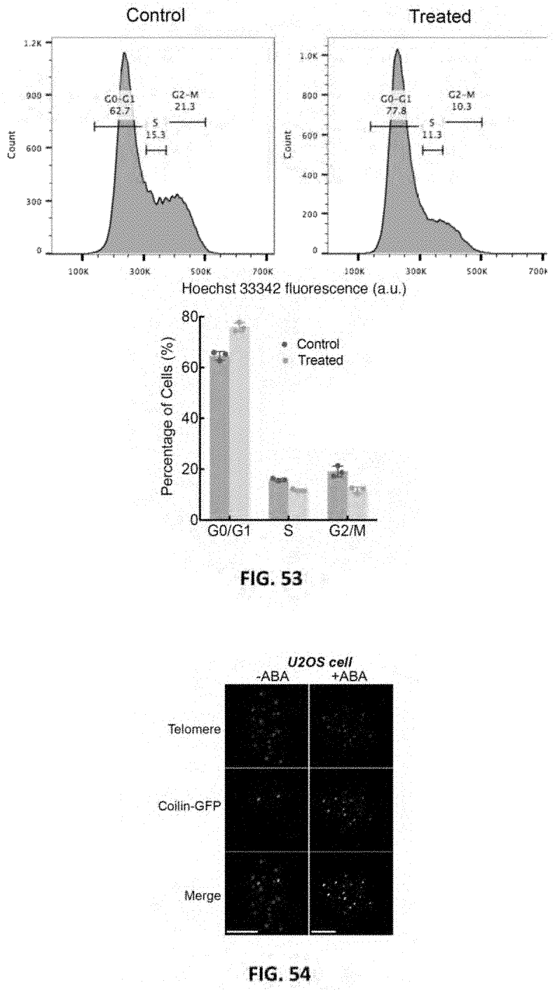

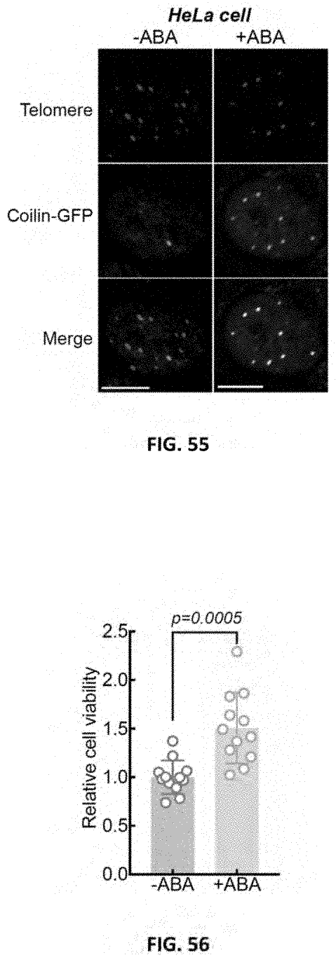

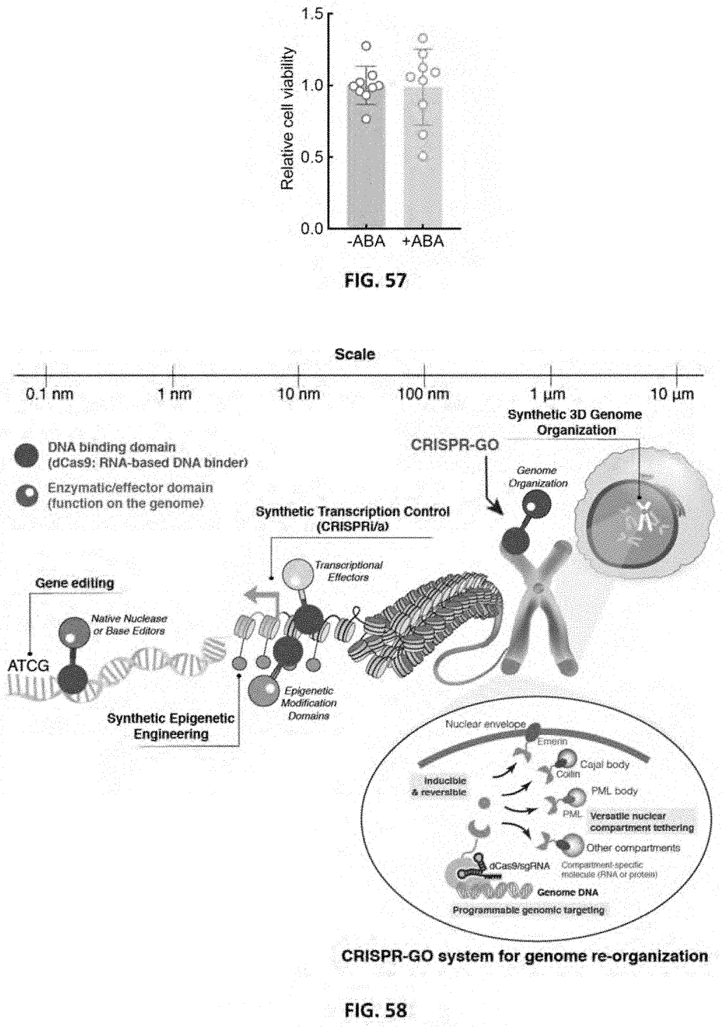

To test this, the CRISPR-GO system was used to disrupt the telomere untethering process during the cell cycle and retain telomeres to the nuclear compartments during interphase (FIG. 51). Using an Alamar blue cell viability assay, which quantifies cell proliferation by measuring metabolic activity of cells, the maintenance of telomeres at the nuclear periphery by CRISPR-GO was found to lead to a significant decrease in cell viability after 6 days of ABA treatment, when compared to untreated cells (FIG. 52, average 72% of reduction, p<0.0001). After 3 days of ABA treatment, cell cycle analysis showed that tethering telomeres to the nuclear periphery increased the percentage of cells in the GO/G1 phase and reduced the percentage of cells in the S phase and the G2/M phase, likely suggesting a GO/G1 phase arrest (FIG. 53). The effect of colocalizing telomeres with CBs was also examined, confirming that the CRISPR-GO system was able to induce colocalization of telomeres and CBs, and finding that the colocalization increased cell viability when comparing cells treated with ABA for 2 days to untreated cells (average 50% increase, FIGS. 54-56). As a control, ABA treatment alone has no effect on cell viability in U2OS cells (FIG. 57). Altogether, these observations suggest that the spatial organization of polynucleotides (e.g., telomeres) relative to various cellular compartments (e.g., nuclear compartments) plays an important role in cellular function.

### Example 13. Plasmids Collections and Constructions

pHR-SFFV-PYL1-sfGFP-Emerin was cloned by replacing scFv sequence in pHR-SFFV-scFv-sfGFP plasmid (Tanenbaum et al., 2014) with PYL1 and inserting Emerin after sfGFP. Emerin (encoded by the EMD gene) was cloned from Emerin pEGFP-C1 (637), a gift from Eric Schirmer (Zuleger et al., 2011) (Addgene plasmid 61993). pHR-SFFV-PYL1-sfGFP-Coilin was cloned by replacing Emerin in pHR-SFFV-PYL1-sfGFP-Emerin plasmid with Coilin. Coilin was cloned from pEGFP-Coilin (Addgene plasmid 36906), a gift from Dr. Greg Matera. pHR-PGK-PYL1-sfGFP-Coilin was cloned by replacing SFFV promoter in pHR-SFFV-PYL1-sfGFP-Coilin plasmid with PGK promoter. pHR-TRE3G-PYL1-sfGFP-PML or pHR-TRE3G-10 PYL1-sfGFP-HP1α was cloned by replacing PGK promoter with TRE3G promoter, and replacing Coilin with PML or HP1α in the pHR-PGK-PYL1-sfGFP-Coilin plasmid. PML was cloned from pLPC-Flag-PML-IV (addgene plasmid 62804), a gift from Gerardo Ferbeyre (Vernier et al., 2011). HP1α was cloned from GFP-HP1α (Addgene plasmid 17652), a gift from Tom Misteli (Cheutin et al., 2003).

pHR-SFFV-ABI-tagBFP-dCas9 was described before (Gao et al., 2016). pHR-SFFV-ABI-tagBFP-dCas9 was cloned by replacing SFFV promotor with PGK promoter pHR-SFFV-ABI-tagBFP-dCas9. pHR-PGK-ABI-dCas9-P2A-Cherry, or pHR-PGK-ABI-dCas9-P2A-Puro was cloned by replacing SFFV with PGK promoter, deleting tagBFP and adding P2A-mCherry or P2A-Puro in dCas9 pHR-SFFV-ABI-tagBFP-dCas9. ABI and PYL1 were cloned from Addgene plasmid 38247 (Liang et al., 2011), a gift from Dr. J. Crabtree, Stanford.

pHR-TRE3G-dCas9-HaloTag was cloned by replacing SunTag10-P2A-mCherry with HaloTag in the plasmid pHR-TRE3G-dCas9-HA-SunTag10-P2A-mCherry (Tanenbaum et al., 2014). pHR-TRE3G-dCas9-EGFP-HaloTag was cloned by inserting HaloTag after EGFP in pHR-TRE3G-dCas9-EGFP (Chen et al., 2013). pHR-SFFV-DHFR-mCherry-Emerin was cloned by replacing PYL1-sfGFP sequence in pHR-SFFV-PYL1-sfGFP-Emerin with mCherry-DHFR. HaloTag and mCherry-DHFR was cloned from pERB221, gift from David Chenoweth & Michael Lampson (Ballister et al., 2014) (Addgene plasmid 61502).

All sgRNAs were cloned into pHR-U6-sgTel-CMV-puro-P2A-mCherry vector after removing P2A-mCherry (Chen et al., 2013). TRF1-mCherry was cloned into pHR-U6-sgTel-CMV-puro-P2A-mCherry vector in place of mCherry. TRF1 was cloned from pLPC-NFLAG TRF1, a gift from Dr. Titia de Lange (Smogorzewska and de Lange, 2002) (Addgene plasmid #16058).

### Example 14. Cell Culture and Generation of Stable Cell Lines

The U2OS (human bone osteosarcoma epithelial, female) cells and Hela cells (female) were cultured in DMEM with GlutaMAX (Life Technologies) in 10% Tet-system-approved FBS (Life Technologies). U2OS 2-6-3 cell line was a gift from Dr. David L. Spector in Cold Spring Harbor Laboratory and were cultured in the same condition (Kumaran and Spector, 2008). All cells were cultured at 37° C. and 5% CO2 in a humidified incubator.

To create stable CRISPR-GO cell lines targeting endogenous loci to nuclear compartments, U2OS cells were plated into 24-well plates 1 day ahead to reach 50% confluency, and then transduced by lentivirus mixture. Cells transduced by lentivirus expressing PYL1-sfGFP-Emerin, PYL1-sfGFP-Coilin, PYL1-sfGFP-PML, or PYL1-sfGFP-HP1α and ABI-tagBFP-dCas9 were sorted by fluorescence activated cell sorting (FACS) at Stanford shared FACS facility for cells that are BFP and GFP positive to create stable cell lines. For nuclear periphery tethering, cells of high BFP and GFP expression level was selected. For other nuclear compartment tethering, cells of high BFP and GFP expression level was selected. After transducing CRISPR-GO cell lines with lentivirus expressing targeting sgRNAs, sgRNA-positive cells were selected with puromycin at 2 μg/ml.

To target LacO loci in the U2OS 2-6-3 cell lines (Kumaran and Spector, 2008), cells were transduced by lentivirus mixture containing PYL1-sfGFP-Emerin or PYL1-sfGFP-Coilin and ABI-dCas9-P2A-mCherry. Cells containing PYL1-sfGFP-Coilin and ABI-dCas9-P2A-mCherry were sorted for GFP and mCherry positive cells to created stable cell lines. SgRNAs positive cells were selected with puromycin at 2 μg/ml.

To quantify the efficacy of LacO nuclear periphery repositioning by CRISPR imaging, U2OS 2-6-3 cells were transduced with lentivirus coding ABI-dCas9-P2A-Puro instead of ABI-dCas9-P2A-mCherry, and were selected with puromycin at 2 μg/ml.

### Example 15. Genomic Loci Targeted by CRISPR-GO System

The efficacy of CRISPR-GO system targeting different chromosomal regions in U2OS cells was tested. Both repetitive regions and non-repetitive genes were tested (FIGS. 7, 14, and 17). The endogenous repetitive regions include Chr3.q29: 195478324-195506987; Ch13.q34: 5 112,277485-112,319169; Ch7:q36.3: 158,122,661-158,135,328; ChX p21.2: 30,806,671-30,824,818 and telomeres. A synthetic LacO repeat inserted in Chr1. p36 region in the U2OS 2-6-3 cells was also used for targeting. For repetitive regions, a single sgRNA design was used targeting multiple repeats within the targeted region (SEQ ID NOs. 1-8 in Table 1). Non-repetitive genes include CXCR4 located at Chr2.q22.1, XIST located at ChrX.q13.2, and PTEN located at Chr10.q23.31. To target each non-repetitive gene, multiple sgRNAs were designed targeting its gene body and upstream region (SEQ ID NOs. 9-36 in Table 2).

TABLE 1
sgRNAs targeting repetitive regions.

| Name | Sequences | Genomic regions |
|---|---|---|
| sgChr3 | TGATATCACAG | Hg38: Chr3: |
| | (SEQ ID NO: 1) | 195478324-195506987 |
| sgChr13 | ACCATTCCTTC | Hg38: CH13: |
| | (SEQ ID NO: 2) | 112277485-112319169 |
| sgChrX | GGCAAGGCAAGGCA | Hg38: ChX: |
| | AGGCACA | 30,788554-30,806701 |
| | (SEQ ID NO: 3) | |
| sgChr7 | GCTCTTATGGTGAG | Hg38: Ch7: |
| | AGTGT | 158,329,969-158342636 |
| | (SEQ ID NO: 4) | |
| sgTel | GTTAGGGTTAGGGT | Telomeres |
| | TAGG | |
| | (SEQ ID NO: 5) | |
| sgLacO-1 | GCTCACAATTCCAC | Chr1.p36 in U2OS |
| | ATG | 2-6-3 cells |
| | (SEQ ID NO: 6) | |
| sgLacO-2 | GCCACATGTGGAAT | Chr1.p36 in U2OS |
| | TGTGAG | 2-6-3 cells |
| | (SEQ ID NO: 7) | |
| sgNT | GTACGTTCTCTATC | Non-targeting |
| | ACTGATATGATATC | |
| | ACAG | |
| | (SEQ ID NO: 8) | |

TABLE 2
sgRNAs targeting non-repetitive regions.

| Name | Sequences | Genomic regions |
|---|---|---|
| PTEN | | |
| sgPTEN-1 | GATCAGCTCTCTCACGGTGAC (SEQ ID NO: 9) | Chr10: |
| sgPTEN-2 | GCACTTGGCTGAGTCCACAGT (SEQ ID NO: 10) | 87,863,113-87,971,930 |
| sgPTEN-3 | GACATCGGAGAATGCACGCTC (SEQ ID NO: 11) | forward strand |
| sgPTEN-4 | GAATAGGTCGATGTAGAGCA (SEQ ID NO: 12) | |
| sgPTEN-5 | GCCGCGTTCTGTAAGAATCGG (SEQ ID NO: 13) | |
| sgPTEN-6 | GTAAGTCCTATGACAGAAGC (SEQ ID NO: 14) | |
| sgPTEN-7 | GAGATATACTGTTAGCGCCTT (SEQ ID NO: 15) | |
| sgPTEN-8 | GGCTGTAGACATCAATGCTT (SEQ ID NO: 16) | |
| sgPTEN-9 | GATCATTGCAGGTAAGAAGTG (SEQ ID NO: 17) | |
| XIST | | |
| sgXIST-1 | GCTCCAACACTCTACCTTGTA (SEQ ID NO: 18) | Chr X: |
| sgXIST-2 | GGAAGTGCTTACGAGTCAAT (SEQ ID NO: 19) | 73,820,651-73,852,753 |
| sgXIST-3 | GCTCTGTCAGTAACTGATAAG (SEQ ID NO: 20) | reverse strand |
| sgXIST-4 | GCAAGCTTACCTGATAGATT (SEQ ID NO: 21) | |
| sgXIST-5 | GTTCCATCTTCTAAGTGTCCT (SEQ ID NO: 22) | |
| sgXIST-6 | GAACTGAGACTTGTGACACA (SEQ ID NO: 23) | |
| sgXIST-7 | GAGTTAGAAGTCTTAAGACC (SEQ ID NO: 24) | |
| sgXIST-8 | GTTCCATCCAACTCAGGCCTT (SEQ ID NO: 25) | |
| sgXIST-9 | GCCTGAGGCTTCTATCTATCT (SEQ ID NO: 26) | |
| sgXIST-10 | GGAAGGCTCATATGGATAGA (SEQ ID NO: 27) | |
| sgXIST-11 | GATCATCTCACATGGCAGCC (SEQ ID NO: 28) | |
| sgXIST-12 | GGCTATCAGAGCAAGCATTG (SEQ ID NO: 29) | |
| sgXIST-13 | GTGTGTGTCATGTGTGGCAG (SEQ ID NO: 30) | |
| CXCR4 | | |
| sgCXCR4-1 | GCGCATGCGCCGCTGGGGCG (SEQ ID NO: 31) | Chr2: |
| sgCXCR4-2 | GCAGACGCGAGGAAGGAGGGCGC (SEQ ID NO: 32) | 136,114,349-136,118,165 |
| sgCXCR4-3 | GGTAGCAAAGTGACGCCGA (SEQ ID NO: 33) | reverse strand. |
| sgCXCR4-4 | GCTCCAGTAGCCACCGCATC (SEQ ID NO: 34) | |
| sgCXCR4-5 | GCCTATATAGTGCGGGTGGG (SEQ ID NO: 35) | |
| sgCXCR4-6 | GTGAGTCGAGGAGAAACGAC (SEQ ID NO: 36) | |

### Example 16. Lentivirus Production

To produce lentivirus, HEK293T cells were transiently transfected with pHR constructs of interest, and packaging plasmids pCMV-dR8.91, and PMD2.G. Lentivirus was collected 72 hours after transfection by filtering supernatant through 0.45 μm filters. When necessary, virus supernatant can be concentrated using Lenti-X concentrator at 4° C. overnight, and centrifuged at 1500 g for 30 min at 4° C. to collect virus pellet. The pellets are suspended in cold culture medium, directly added into cells or frozen down in −80° C.

### Example 17. Genomic Loci Visualization Via CRISPR Imaging and FISH

CRISPR imaging was performed to visualize the localization of Chr3, Chr13 and LacO loci in living cells (FIG. 5). For live-cell CRISPR imaging, stable cell lines expressing CRISPR-GO components were transduced with lentivirus coding dCas9-HaloTag and targeting sgRNAs in ibidi 24-well microplate (Ibidi.inc). Targeted genomic loci are labeled by dCas9-HaloTag and stained by JF549-HaloTag ligand at 0.1-0.5 μM for 15 min at 37° C. in culture media. After staining, cells were washed with culture medium twice, and then incubated in phenol-red free culture medium during microscopy. JF549-HaloTag was a gift from Dr. Luke D Lavis in Janelia Research Campus (Grimm et al., 2015). Telomere loci are labeled in living cells by expression of TRF1-mCherry, a telomere binding protein.

Other genomic loci are labeled by DNA FISH in fixed cells. Cells were grown in ibidi chamber slides with a removable 12 well silicone chamber, and fixed with 4% PFA for 20 minutes. Lac 0, Chr7 and ChrX loci were labeled using synthesized fluorescent nucleotide probes (Integrated DNA Technologies, Redwood City, Calif.) according to a FISH protocol described (Takei et al., 2017). LacO loci were labeled with the Alexa Fluor 647 labeled FISH probe 5′-TTGTTATCCGCTCACAATTCCACATGTGGCCACAAA-3′ (SEQ ID NO: 40) at 10 nM concentration. Chr7 loci were labeled by Cy3 labeled FISH probe 5′-Cy3-CCCACACTCTCACCATAAGAGC-3′ (SEQ ID NO: 41) at 200 nM, and ChrX loci were labeled by 5-Cy3-TTGCCTTGTGCCTTGCCTTGC-3′ (SEQ ID NO: 42) at 200 nM. The CXCR4 FISH probe was purchased from Empire Genomics. The PTEN and XIST FISH probes were purchased from Cell Line Genetics. FISH was performed according merchandiser's protocols.

### Example 18. Immunostaining of Nuclear Body Markers

To detect co-localization between Cajal body markers and targeted LacO loci, U2OS 2-6-3 cells expressing a low level of PYL1-sfGFP-Coilin were transfected with lentivirus coding PGK-ABI-dCas9-P2A-Puro and sgLacO on day 0, treated with puromycin and 3 mM ABA on day 1, and fixed on day 2 after 20 hours of ABA treatment. FISH was performed in fixed samples to detect LacO loci using Alexa Fluor 647 labeled FISH probe, and then immunostaining was performed using mouse monoclonal anti-SMN, anti-Fibrillarin and anti-Gemin2 antibody, and Donkey anti-mouse Alex Fluor 594 secondary antibody.

To detect co-localization between PML body markers and targeted Chr3 loci, U2OS cells expressing PYL1-sfGFP-Coilin and PGK-ABI-dCas9 were transfected with lentivirus coding dCas9-HaloTag (for CRISPR imaging) and sgChr3 on day 0, treated with puromycin and 3 mM ABA on day 1, stained by JF549-HaloTag and fixed in 4% paraformaldehyde (PFA) in Day 3. Immunostaining was performed in fixed samples with rabbit polyclonal anti-SP100, and Donkey anti-rabbit Alex Fluor 647 secondary antibody.

For immunostaining, the fixed samples permeabilized in the permeabilization buffer (PBS, 1% Triton-X100) for 15 min, blocked in blocking buffer (PBS, 0.3% Triton-X 100, 5% Donkey normal Serum) for 1 hour, incubated with the primary antibody diluted in the blocking buffer overnight at 4° C., washed in PBS three times, then incubated with the secondary antibody at room temperature for 1-2 hours, and washed four times in PBS.

### Example 19. Chemical Induction and Reversal Experiments

For re-localization experiments, U2OS cells containing chemical-inducible re-localization systems and sgRNAs are treated by abscisic acid (ABA, Sigma-Aldrich, A1049) at 3 mM for 2 days before imaging or fixation.

For the time-course chemical induction experiment targeting Chr3 to nuclear periphery, U2OS cells containing CRISPR-GO and CRISPR imaging systems and sgRNAs targeting Chr3 were treated with or without 3 mM ABA, stained by JF549-HaloTag, and fixed at different time points. For the time-course reversal experiment, the Chr3-targeting U2OS cells were pre-treated with 3 mM ABA for 2 days, washed five times, and switched to medium without ABA. Cells were stained by JF549-HaloTag ligand for CRISPR imaging and fixed in 4% paraformaldehyde for 20 min at different time points.

For the time-course chemical induction experiment targeting LacO to Cajal body, U2OS 2-6-3 cells expressing a low level of PYL1-sfGFP-Coilin were transfected with lentivirus coding PGK-ABI-BFP-dCas9 and sgLacO on day 0, treated with puromycin on day 1, treated with or without 3 mM ABA on day 2 and fixed after 30 minutes of ABA treatments. For the time-course reversal experiment, cells were pre-treated with 3 mM ABA for 2 days, washed five times, and switched to medium without ABA. Cells were fixed in 4% paraformaldehyde for 20 min at different time points.

### Example 20. Cell Cycle Synchronization

To dissect mitosis-dependence effect of genomic re-localization, U2OS cells containing CRISPR-GO and CRISPR imaging systems and sgRNAs targeting Chr3 were used for this experiment. On day −3, cells were starved in 0.5% FBS in medium for 2 days. On day −1, cells were switched to normal growth medium with 10% FBS and treated with 2 mM hydroxyurea (HU) for G1/S phase blockage for 1 day. On day 0, while keeping the HU treatment, cells were treated with or without ABA. Control cells were treated in the same way but without HU. Cells were stained by JF549-HaloTag for CRISPR imaging and fixed in 4% paraformaldehyde 24 h or 48 h after ABA treatment.

### Example 21. Microscopy

With the exception of FIG. 22, all microscopy was performed on a Nikon TiE inverted confocal microscope equipped with the 100×PLAN APO oil objective (NA=1.49), 60×PLAN APO oil objective (NA=1.40) or the 60×PLAN APO IR water objective (NA=1.27), an Andor iXon Ultra-897 EM-CCD camera and 405-nm, 488-nm, 561-nm and 642-nm lasers. Images were taken using NIS Elements version 4.60 software by time-lapse microscopy with Z stacks at 0.2-[tm or 0.4-[tm steps. For live cell imaging, cells were kept at 37° C. and 5% CO2 in a humidified chamber.

For long-term live cell imaging shown in FIG. 22, microscopy was performed in Leica DMI8 inverted microscope equipped with the 63×HC PLAN APO oil objective (NA=1.40), a Leica DFC9000 CT camera and a Lumoncor SOLA SM II 405 light source. Images were taken using LAS X Software by time-lapse microscopy every 30 minutes for 20 hours, using GFP and TXR filter cubes. During imaging, cells were kept at 37° C. and 5% CO2 in a humidified chamber (Okolab Cage incubation system).

To visualize the dynamics of chromatin-Cajal body association in individual cells (FIGS. 38-41, 44, and 45), U2OS 2-6-3 cells expressing a lower level of PYL1-sfGFP-Coilin was transfected with lentivirus coding PGK-ABI-BFP-dCas9 and sgLacO on day 0, treated with puromycin on day 1 and seeded in ibidi 96 well u-plates. Each well was imaged under confocal microscope to focus on a ABI-BFP-dCas9 labeled LacO locus in a chosen cell. Images were captured before ABA treatment for comparison. Without moving the sample under the confocal microscope, 10-fold ABA-containing culture medium was added into the imaging well to reach a final concentration of 1 mM ABA, and then the same cell containing the previously focused LacO locus was immediately imaged after adding the ABA. The first image taken after ABA addition was given t=0, and all other images were aligned by the capture time accordingly.

### Example 22. Imaging Processing and Data Analysis

Image processing was performed in Fiji (image J) (Schindelin et al., 2012) or MetaMorph (Molecular devices, CA). A single microscope plane showing maximum fluorescence of labeled genomic loci, or the average of two/three adjacent Z planes showing maximum loci fluorescence are shown in the drawings herein. Some images were processed using the “smooth” function in Fiji to reduce noises for visualization only.

Line scan was performed using the “Analyze/Plot Profile” function in Fiji, analyzed in Excel and plotted in GraphPad Prism (Version 7.00 for Mac OS, GraphPad Software, La Jolla Calif. USA, www.graphpad.com). Fluorescence intensity at each point along the line were normalized relative to the maximum (=1) and the minimum (=0) fluorescence intensity along the line.

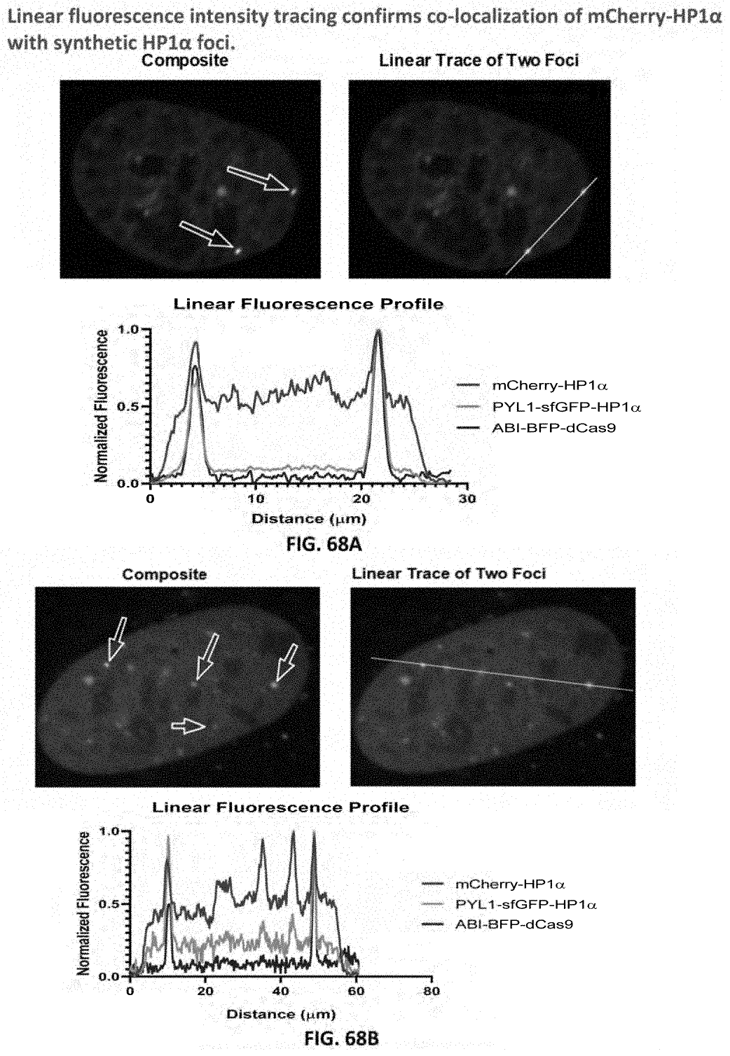

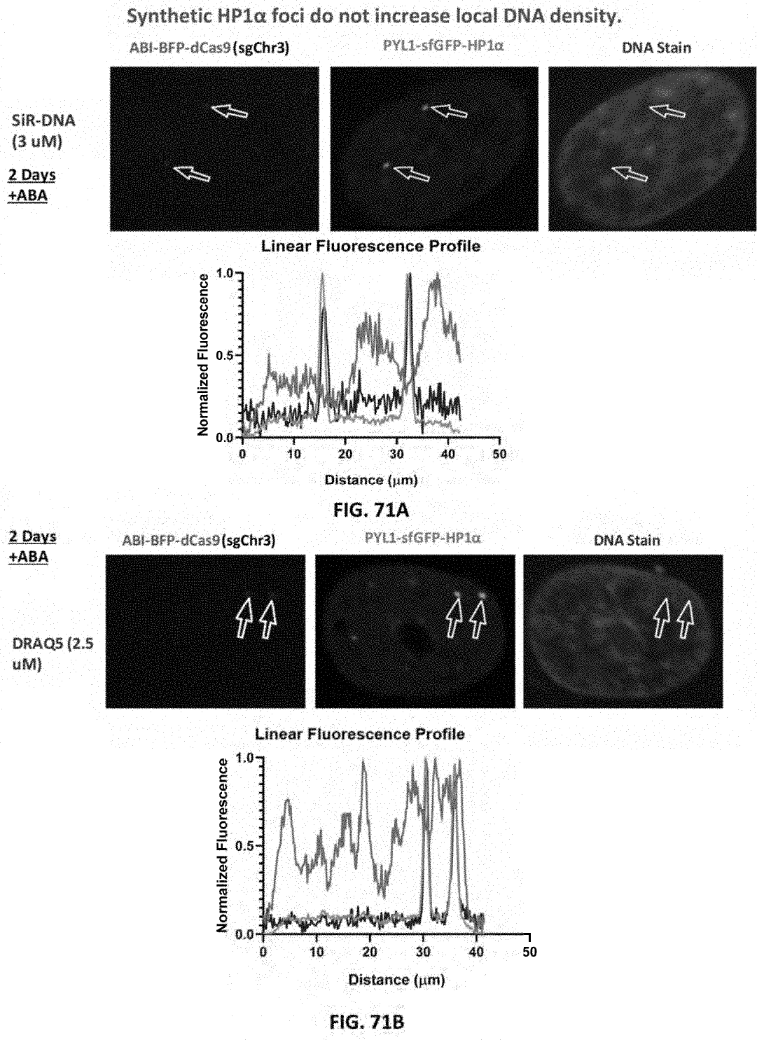

Linear tracing to evaluate co-localization of target proteins on cells (FIGS. 68, 70-73, 75-80, 85, 89 and 92) was performed using FIJI/ImageJ (version 1.52) software on the presented images, which represent single microscope planes at the maximum fluorescence of labeled genomic loci. A line was drawn between two synthetic foci using the “Straight Line” function in FIJI, with endpoints extended outside of the cell nucleus. If more than two synthetic foci were visible, two were arbitrarily chosen. If only a single synthetic focus was visible, the line extends in an arbitrary direction. The raw fluorescence intensities for all channels along the line are plotted using the “Analyze/Plot Profile” function in FIJI. The raw intensity at each point along the line was normalized to the maximum (1) or the minimum (0) fluorescence intensity measured along the line.

### Example 23. Quantification of Genomic Repositioning Events

To determine the peripheral recruitment efficacy in living U2OS cells, Chr3, Chr13 and Chr1/Lac0 loci are labeled by CRISPR imaging and telomeres are labeled by TRF1-mCherry, while the nuclear membrane is labeled by PYL1-sfGFP-Emerin. After scanning Z-stacks of confocal planes, the position of each labeled locus is viewed in slice viewer (NIS element viewer) to determine its position in XY, XZ and YZ planes. Without double counting any loci, the loci were categorized into three categories: loci located directly in the nucleus periphery that co-localize with PYL1-GFP-Emerin in XY, YZ and YZ planes, loci that do not co-localize with PYL1-GFP-Emerin, and loci that co-localize with internal PYL1-GFP-Emerin not at nuclear periphery (in rare cases). The number of loci in each category was recorded for each individual cell. Only loci of the first category that co-localize with PYL1-GFP-Emerin at the nuclear envelope were counted as nuclear periphery positioned loci. Cells containing at least one nuclear periphery positioned loci were quantified.

To determine peripheral recruitment efficacy in fixed U2OS cells (e.g., Chr7, ChrX, PTEN, CXCR4, XIST), targeted genomic loci are labeled by FISH and the nucleus are stained by DAPI. After scanning Z-stacks of confocal planes, the position of each labeled locus is viewed in 3D space to determine its position in XY, XZ and YZ planes. A genomic locus that located at the edge of nucleus (DAPI) in 3D space is categorized as a periphery-located locus. Otherwise it is considered as an internal-located locus. The number of loci in each category was recorded for each individual cell. Cells containing at least one nuclear periphery positioned loci were also quantified.

To detrmine the Cajal body co-localizing efficacy in fixed U2OS 2-6-3 cells, targeted LacO loci were labeled by FISH, nuclei were stained by Hoechst 33342, and Cajal bodies were labeled by PYL1-GFP-Coilin. After scanning Z-stacks of confocal planes, we identified the position of each LacO locus in 3D space. Without double counting, the loci were categorized two categories: loci that co-localize with PYL1-GFP-Coilin, and loci that do not co-localize with PYL1-GFP-Coilin. The number of loci in each category was recorded for each individual cell. Cells containing at least one Cajal body-co-localized loci were also quantified.

### Example 24. Fluorescence Assays Using Flow Cytometry Analysis

For quantification of CFP-SKL expression, U2OS 2-6-3 cells containing ABI-dCas9-P2A-mCherry and PYL1-sfGFP-Emerin or PYL1-sfGFP-Coilin were transduced with sgRNA targeting lacO loci or non-targeting sgRNAs, treated with ABA at 3 mM for 2 days and then induced with doxycycline at 50 ng/ml for 40 hours (nuclear periphery tethering) or 24 hours (Cajal body tethering). After the treatment, U2OS 2-6-3 cells were dissociated using 0.25% Trypsin EDTA (Life Technologies) and analyzed by flow cytometry on CytoFlex S (Beckman Coulter Life Sciences) using 405-nm, 488-nm and 561-nm lasers. At least 8,000 cells were analyzed for each sample. Cells were gated for positive dCas9 (mCherry) and Emerin (GFP) expression. CFP-SKL fluorescence was detected using the 405 nm laser and 450/45 filter. To quantify relative fluorescence, the average total fluorescence of untreated (without Dox and ABA) cells is set to 0, while the average total fluorescence of doxycycline induced cells (with Dox only) is set to 1. Technical replicates in 3 independent experiments are reported.

### Example 25. Real-Time RT-PCR for Endogenous Gene Expression

Real-time RT-PCR were performed to determine the expression change in PPP1R2 TFRC and ACAP2 gene adjacent to targeted Chr3 loci after genomic re-organization. For each sample, total RNAs were isolated using RNeasy Plus Mini Kit (Qiagen Cat 74134) and cDNAs were synthesized using the iScript cDNA Synthesis Kit (BioRad, Cat 1708890), according to manufacturer's protocols. Quantitative PCR was performed using the PrimePCR assay with the SYBR Green Master Mix (BioRad), and run on Biorad CFX384 real-time system (C1000 Touch Thermal Cycler), according to manufacturer's instructions. Cq values was used to quantify gene expression. The relative expression of the PPP1R2, TFRC, and ACAP2 genes was normalized to GAPDH control. To calculate the relative mRNA expression level, the relative expression of each treatment was normalized by setting the average value in non-ABA treated samples as 1. Replicates in 3 experiments are reported.

### Example 26. Cell Viability Assay

Cell viability assay was performed using Alamar blue cell viability reagents (ThermoFisher Scientific), which measures the metabolic activity of the cells. For each condition, 100 μl cells treated with and without ABA were seeded at equal concentration (500-1000 cells/well) in the same 96-well plate. At the time of detection, 10 μl of Alamar blue reagents were added to each well and the plates were incubated at 37° C. for 1 hour. After that, the fluorescent intensity was measured in the Synergy H1 microplate reader (Biotek Inc.) using the excitation wavelength at 540 nm and the emission wavelength at 585 nm. Average fluorescent intensity of wells containing only 100 μl culture medium (with and without ABA) was used as blanks. For each well, the relative fluorescent intensity is calculated by subtracting background (average intensity of blank wells) from its raw fluorescent intensity. To calculate the relative cell viability, the relative florescent intensity in each well was normalized by setting the average value in non-ABA treated wells as 1. Replicates in 3 experiments are reported.

### Example 27. Cell Cycle Cytometry Analysis

To quantify how telomere nuclear periphery tethering affect cell cycle progression, U2OS cells containing nuclear periphery tethering system were treated with lentivirus mixtures coding sgTelomere and TRF1-mcherry, or lentivirus coding a non-targeting sgRNA. Telomere tethering was confirmed by microscopy after 2 days of ABA treatment. After 3 day of ABA treatment, control and treated cells were dissociated using 0.25% Trypsin EDTA, with stained Hoechst 33342 at 1:1000 dilution for 1 h, and analyzed by flow cytometry on CytoFlex S (Beckman Coulter Life Sciences) using 405-nm lasers. At least 20,000 cells were analyzed for each sample. Cell cycle analysis was performed using FlowJO.

### Example 28. Identification of Human Repetitive Sequence Clusters

The software Tandem Repeats Finder (Benson, 1999) was used to identify all tandem repeats of 14-nucleotides or longer sequences from the human genome (hg38). Regions that contain ten or more identical tandem repeats were defined a “repetitive sequence cluster.” These repetitive sequence clusters were to each human chromosome. Distances between the repetitive sequence clusters and genes were calculated using the BEDTools suite.

### Example 29. Short-Term Dynamic Tracking of Endogenous Loci

Genomic loci tracking was performed using the TrackMate plugin (Tinevez et al., 2017) in Fiji. For tracking genomic loci, the estimated blob diameter was set between 0.5-1. Linking max distance was set to 2 and gap closing distance was set to 3 μm and gap closing max frame was set to 2. Position of each locus (x^{t}, y^{t}) at different time point (t) were measured, analyzed in Excel and plotted in GraphPad Prism 7. The movement step (dx, dy) was calculated by subtracting the position of a previous time point from the new position: dx^{t}=x^{t}−x^{t-1} & dy^{t}=y^{t}−y^{t-1}, where (x^{t},y^{t}) is position of a locus at time t, while (x^{t-1}, y^{t-1}) is the position of the locus at the previous time point (t−1). Step distance=√((x^{t}−x^{t-1})²+(y^{t}−yₜ₋₁)²) is calculated as how far a locus move away from its position at the previous time point.

To compare step distances, 1696 step distances of 19 interior-localized Chr3 loci and 1669 step distances of 14 periphery-localized Chr3 loci were analyzed. The two-side t-test with unequal variance was performed. Histogram were analyzed using Histogram function in Excel and plotted in in GraphPad Prism 7.

### Example 30. Statistics Analysis

For quantification of re-localization efficacy (FIGS. 8, 9, 16, 18-21, 30, 33, 36, 38, and 39), p value was calculated using Fisher's exact test in GraphPad, and error bars show standard error of the mean (SEMs) calculated according to Bernoulli distributions. The numbers of counted loci/cells are listed at the bottom of each figure. For FIGS. 26, 42, 43, 46-51, 53, 56, and 57, p value was calculated using two-sided t-test with unequal variance in Excel and error bars show standard deviations. Duplicates in 3 experiments were analyzed. For FIG. 27, fit Gamma distributions were fit by maximum likelihood using the R package fitdistrplus and tested the goodness of fit using the Kolmogorov-Smirnov test (p=0.06 for periphery loci & p=0.77 for interior loci).

Although the foregoing invention has been described in some detail by way of illustration and example for purpose of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### Example 31. Homology Directed Repair

To implement an inducible CRISPR-mediated chromatin repositioning system to facilitate homology directed repair (HDR), a chemical-inducible heterodimerization system is tested. This system is an abscisic acid (ABA) inducible ABI/PYL1 system. The Streptococcus pyogenes dCas9 (D10A & H840A) protein is fused to one heterodimer, and various proteins that facilitate HDR, such as Rad51, Rad52, and the MRN complex, are fused to the cognate heterodimer. A template polynucleotide to be used for HDR is also fused to the cognate heterodimer. U2OS human bone osteosarcoma epithelial cell lines are created using lentiviral transduction that stably expressed the dimerization system. In these cell lines, spatial re-localization of HDR fusion proteins to the ABI-BFP-dCas9 protein is caused by addition of ABA, due to its dimerization with PYL1-GFP-HDR proteins. A CRISPR/Cas9 complex that generates a double stranded break in the target polynucleotide of the ABI-BFP-dCas9 protein is introduced into the cells, and a double stranded break in the target polynucleotide is generated. The double stranded break in the target polynucleotide is preferentially repaired by HDR.

### Example 32: Generation of Large-Scale Heterochromatin Domains at Targeted Genomic Loci

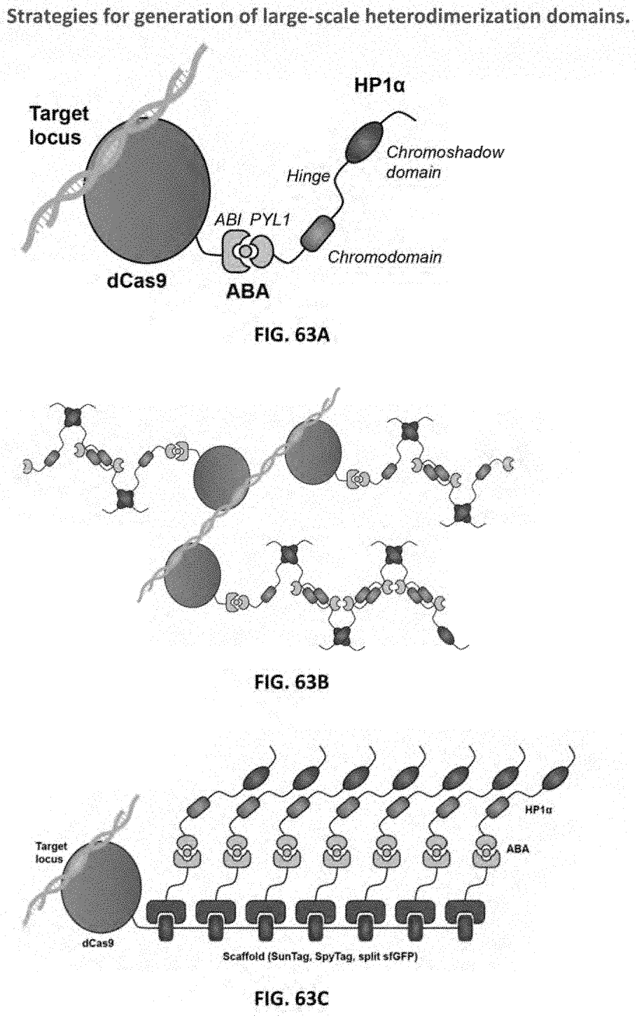

This example shows the generation of heterochromatin domains at a targeted genomic locus. An inducible dCas9 and a heterochromatin protein are fused to complementary pairs of heterodimerization domains, which assemble in the presence of the chemical inducer. FIG. 63 is a schematic illustration of a programmable, inducible, and versatile systems for generation of high concentrations of heterochromatin proteins at genomic loci by targeting tandem repeat regions.

In order to generate high concentrations of heterochromatin proteins at target sites, an inducible ABI-PYL1 heterodimer system is used. FIG. 63A shows that an inducible dCas9 system (d-Cas9 linked to ABI) recruits heterochromatin proteins such as HP1α (PYL1 linked to HP1α) to target sites via heterodimerization of the ABI and PYL1 domains upon addition of the plant hormone abscisic acid (ABA). The genomic target polynucleotide are specified by the sgRNA, which form a complex by binding to the dCas9 and then direct dCas9 to the genomic target polynucleotide by binding to the genomic target polynucleotide. FIG. 63B shows that a single sgRNA that targets a genomic polynucleotide that is a tandem repeat allows potential binding of multiple (e.g., tens, hundreds, etc.) of genomic sites in close proximity to one another. Furthermore, HP1α spontaneously forms homodimers via its chromoshadow domain and has been reported to oligomerize via N-terminus to hinge region interactions, further increasing the concentrations of heterochromatin proteins at target sites. These mechanisms enable a single HP1α to recruit additional HP1α to the genomic target polynucleotide. FIG. 63C shows the number of proteins recruited to a genomic target polynucleotide is further increased on a per actuator moiety basis by engineered protein scaffolds, such as by increasing the number of heterochromatin proteins bound to a dCas9 of the this system. Numerous copies of the heterochromatin proteins are recruited to a target location by linking the dCas9 to a scaffold, such as a peptide array or such as a SpyTag, SunTag split sf-GFP, MoonTag, SnoopTag, split sfCherry2, or mNeonGreen2 protein scaffolds, which can serve as protein multimerization domains.

### Example 33: Recruitment of Engineered Heterochromatin Protein HP1α to Chr3 Target Site

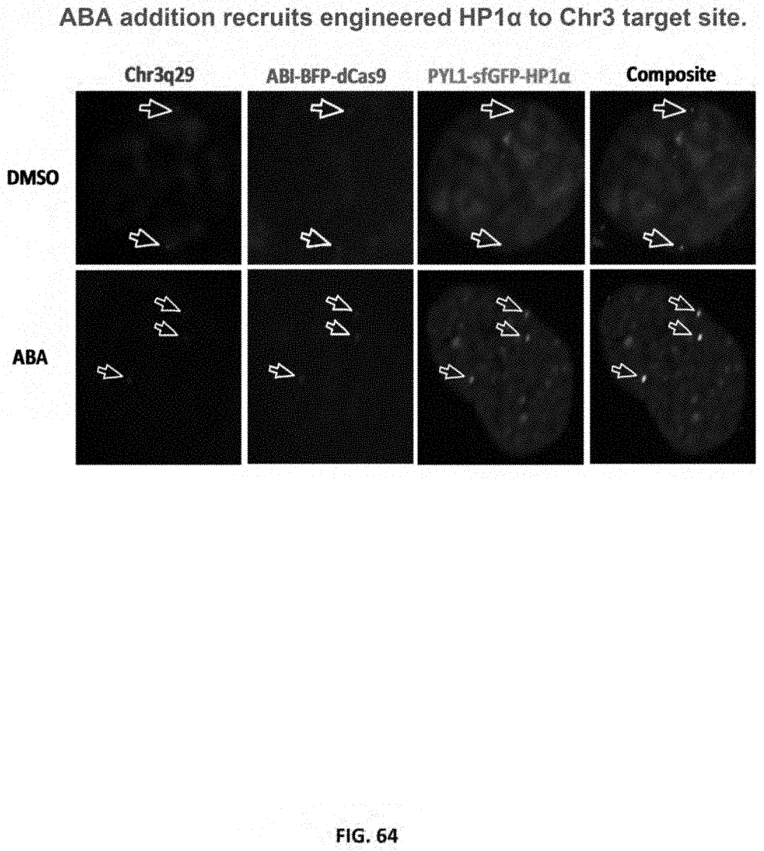

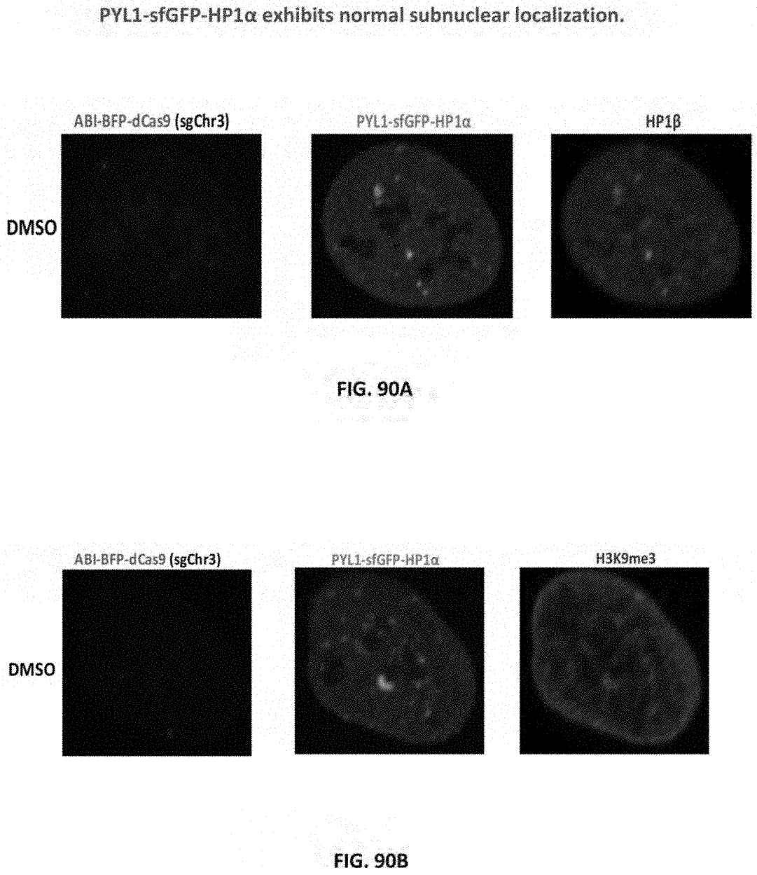

This example shows the dynamic recruitment of engineered heterochromatin protein HP1α to a target site on Chromosome 3. In order to generate heterochromatin protein HP1α to the target sites, an inducible ABI-PYL1 was used to effect dimerization of the protein complex upon addition of abscisic acid. dCas9-HaloTag, ABI-BFP-dCas9, PYL1-sfGFP-HP1α, and sgChr3q29 were stably integrated into U2OS human osteosarcoma cells by lentiviral transduction (sgChr3q29: SEQ ID NO: 1; target PAM sequence for sgChr3q29: TGG (SEQ ID NO: 37)). FIG. 64 shows representative microscopic images showing colocalization (arrows) of targeted Chr3:q29 loci (left panel by CRISPR-Cas9 imaging), ABI-BFP-dCas9 (middle panel), PYL1-sfGFP-HP1α labeled (third panel) and a composite image (right panel) with or without ABA. In the absence of abscisic acid, PYL1-sfGFP-HP1α localized to natural heterochromatin sites in the nucleus but not at Chr3q29. FIG. 90 shows that PYL1-sfGFP-HP1α exhibited normal subnuclear localization in the absence of ABA. U2OS cells stably expressing ABI-BFP-dCas9, PYL1-sfGFP-HP1α, and sgChr3q29 were imaged after 2 days of DMSO vehicle treatment. Cells were immunostained with HP1β or H3K9me3 primary and AlexaFluor647 secondary antibodies. In the absence of ABA inducer, PYL1-sfGFP-HP1α localized to regions of natural heterochromatin, defined by the presence of HP1β and H3K9me3 localization.

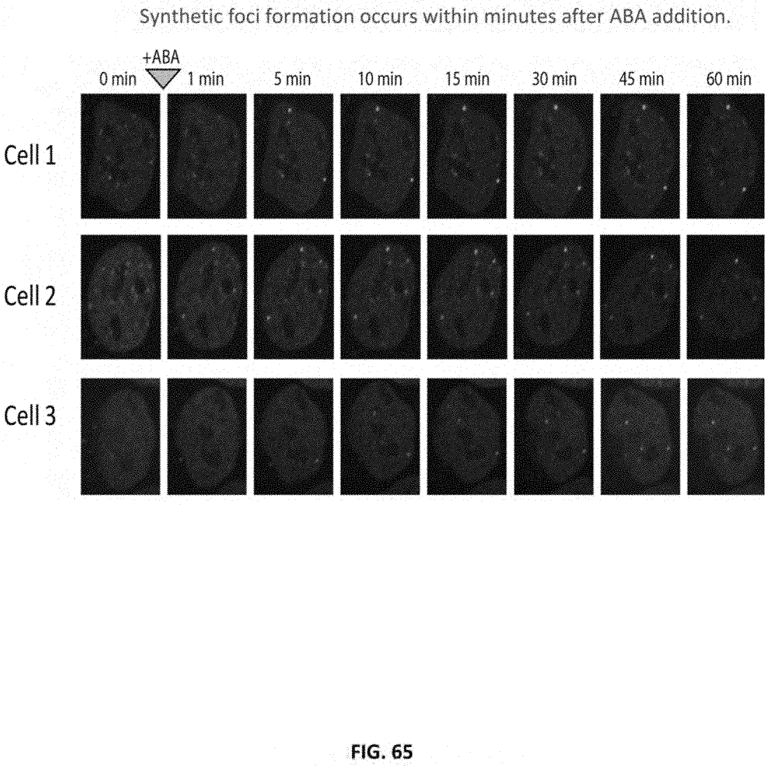

Upon treatment with ABA, PYL1-sfGFP-HP1α formed strongly fluorescent foci at the Chr3q29 locus. Time lapse confocal microscopy revealed that PYL1-sfGFP-HP1α recruitment and foci formation occurred as early as 1-10 min after ABA addition. FIG. 65 shows representative real-time microscopic images (0-60 minutes) showing the rapid formation of de novo heterochromatin loci of PYL1-sfGFP-HP1α (white loci) after ABA addition. The nuclear localization of PYL1-sfGFP-HP1α was tracked immediately before and for 60 min after addition of 100 μM ABA. The chosen cell was imaged first before ABA treatment (0 min). ABA was added to the culture medium between 0 min and 1 min, and 1 min represents the first image taken of the same cell immediately after ABA addition. The timing of foci formation for three representative cells are shown.

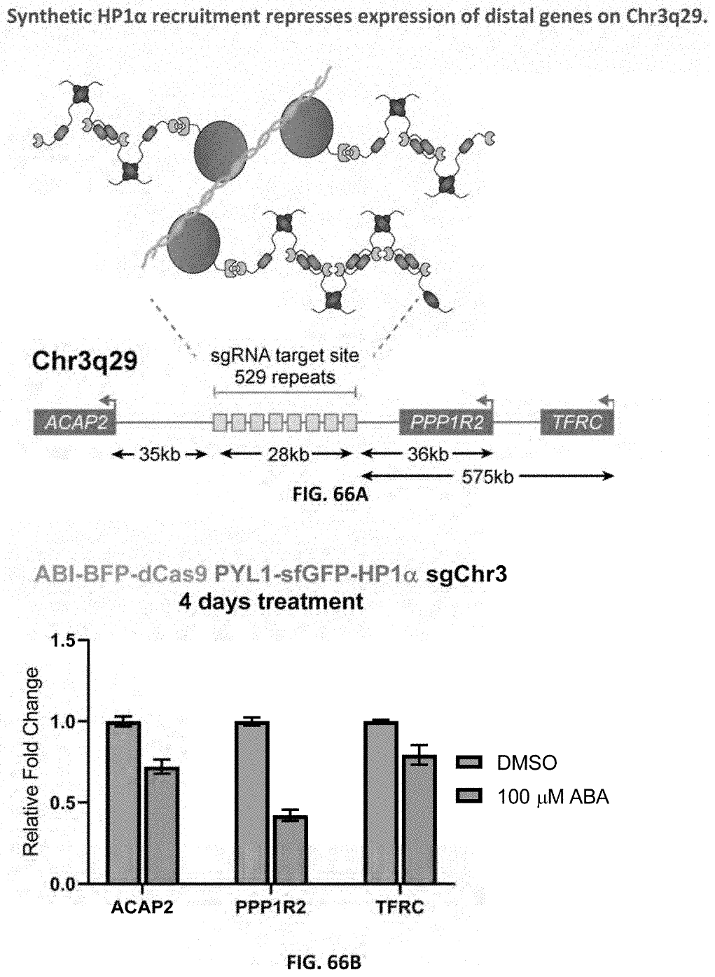

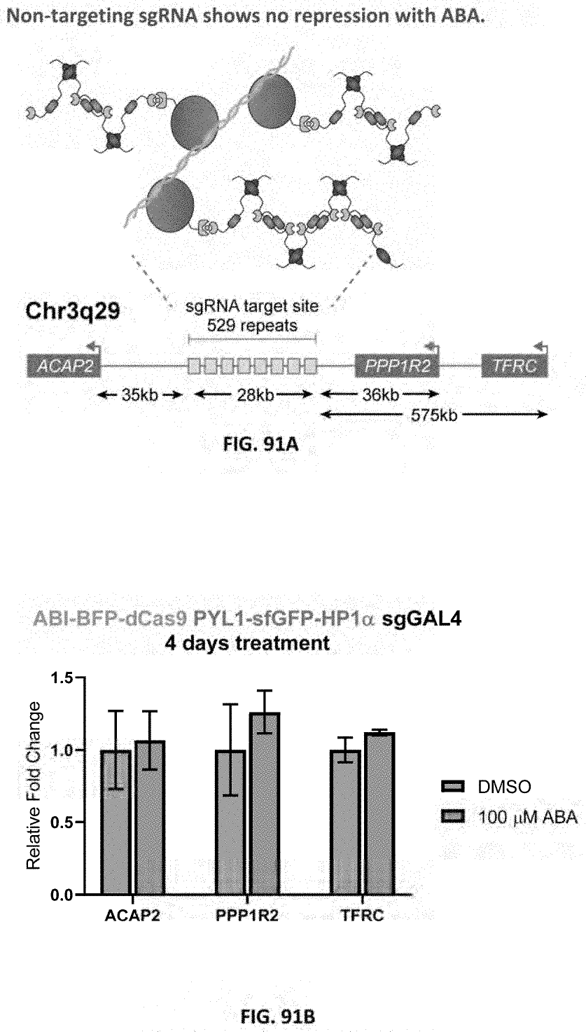

Furthermore, expression of distal genes on Chr3q29 was repressed by recruitment of PYL1-sfGFP-HP1α. Expression of three genes on Chr3q29 at between 35 kb and 575 kb flanking the tandem repeat target site were quantified by RT-qPCR after 4 days treatment with either 100 μM ABA or DMSO vehicle. FIG. 66 shows that recruitment of PYL1-sfGFP-HP1α significantly reduced expression of all three genes. FIG. 66A shows the schematic illustration of the CRISPR-GO system by recruitment of PYL1-sfGFP-HP1α to the Chr3:q29 locus in U2OS cells. ACAP2 is located ˜35 kb upstream of the sgRNA target site, and PPP1R2 is located ˜36 kb downstream of the sgRNA target site. TFRC is located about 575 kb downstream of the sgRNA target site. FIG. 66B shows the graph of comparison of ACAP2, PPP1R2, and TFRC gene expression (measured by RT-qPCR) using CRISPR-GO to colocalize PYL1-sfGFP-HP1α to the Chr3:q29 loci in +/− ABA conditions. Data are represented as mean±SD. FIG. 91 shows no repression of endogenous gene expression adjacent to targeted loci and across long distances by recruitment of a non-targeting sgRNA to Chr3q29 locus, and thus also in the absence of a PYL1-sfGFP-HP1α sgChr3. A control experiment showed that addition of 100 μM ABA with a non-targeting PYL1-sfGFP-HP1α sgGAL4 (sgGAL4: guide RNA that does not target the Chr3q29 tandem repeat site) did not induce repression of the Chr3q29 genes. Gene expression was quantified by RT-qPCR 4 days after 100 μM ABA or DMSO vehicle treatment.

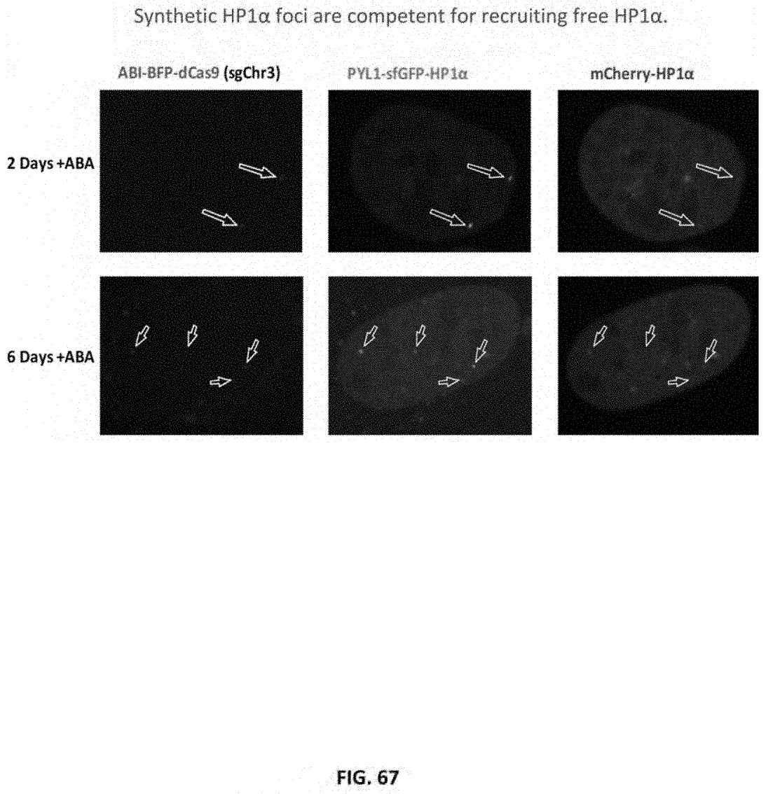

Furthermore, free-floating HP1α is recruited to synthetic HP1α foci. Natural heterochromatin recruits heterochromatin protein 1 (HP1) family proteins HP and HP1(3. FIG. 67 show that synthetic HP1α foci are competent for recruiting free HP1α. Free-floating mCherry-HP1α (right panel) was recruited to the synthetic heterochromatin foci (arrows) formed by PYL1-sfGFP-HP1α (middle panel) and ABI-BFP-dCas9 (left panel), after 2 days and 6 days of ABA treatment. At both 2 and 6 days after 100 μM ABA treatment, free-floating HP1α foci tagged with mCherry were observed to be enriched at the synthetic heterochromatin foci formed by PYL1-sfGFP-HP1α.

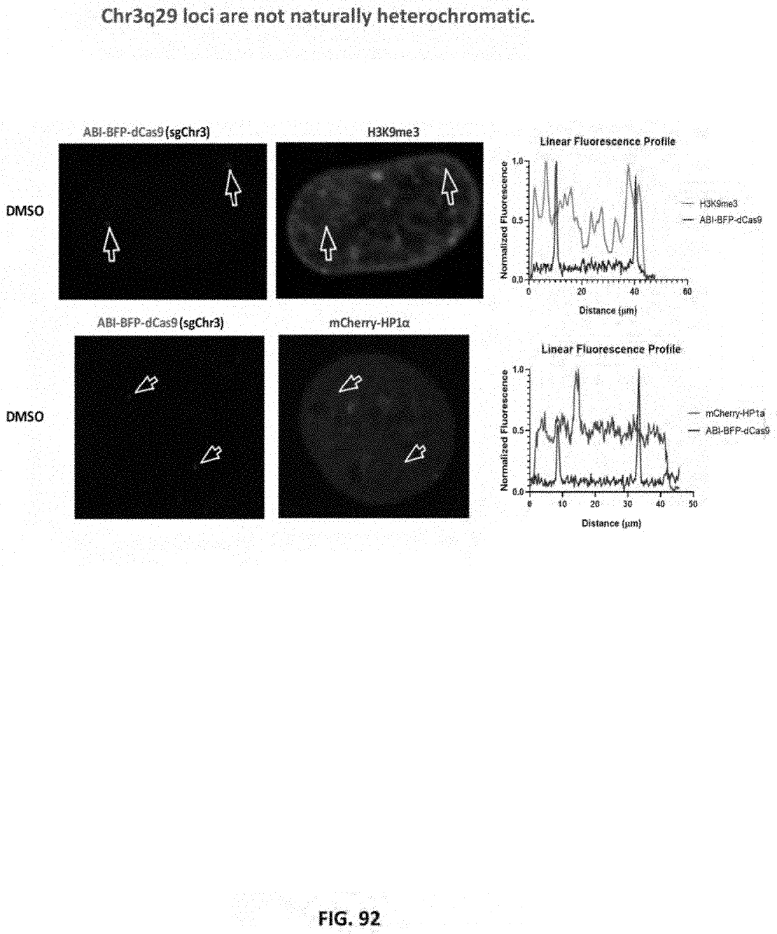

FIG. 92 shows that Chr3q29 loci are not naturally heterochromatic. FIG. 92 shows lack of H3K9me3 colocalization (middle panel, top) or ABI-BFP-dCas9 localization (sgChr3) (left panel, top and bottom) with mCherry-HP1α (middle panel, bottom) in the presence of DMSO, and thus also in the absence of the ABA inducer. Corresponding normalized linear fluorescence profile of the co-localization profile of HP1α with heterochromatin shows lack of selective enrichment of HP1α at peaks of H3K9me3 and ABI-BFP-dCas9 (graphs). For the top graph, the light gray higher line is for H3K9me3 and the dark lower line is for ABI-BFP-dCas9. For the bottom graph, the light gray higher line is for mCherry-HP1α and the dark lower line is for ABI-BFP-dCas9.

Linear fluorescence intensity tracing was used to confirm the co-localization of mCherry-HP1α with synthetic HP1α foci. FIG. 68A and FIG. 68B shows fluorescence images of composite images (left panel) showing co-localization of mCherry-HP1α loci and synthetic HP1α foci (indicated by arrows). The right panel shows the linear trace of two foci for each image with a line. Normalized linear fluorescence profile of the co-localization of the mCherry-HP1α with synthetic heterochromatin puncta show selective enrichment at peaks of PYL1-sfGFP-HP1α and ABI-BFP-dCas9 (graphs). For both graphs, the top dark line is mCherry-HP1α, the light gray line is PYL1-sfGFP-HP1α, and the bottom gray line is ABI-BFP-dCas9. Linear trace of the two foci (white lines in the top middle and bottom middle panels), were drawn to intersect two given synthetic heterochromatin puncta from nuclear edge to edge. For these figures, the fluorescence intensities for mCherry-HP1α, PYL1-sfGFP-HP1α, and ABI-BFP-dCas9 were plotted along a line drawn to intersect two given synthetic heterochromatin puncta from nuclear edge to edge. Linear fluorescence profile tracing shows that mCherry-HP1α colocalizes with synthetic heterochromatin puncta as shown by the selective enrichment of mCherry-HP1α peaks at peaks of PYL1-sfGFP-HP1α and ABI-BFP-dCas9 (top right and bottom right panels). Fluorescence values were normalized with 1 being the maximum fluorescence and 0 being the minimum fluorescence along the line.

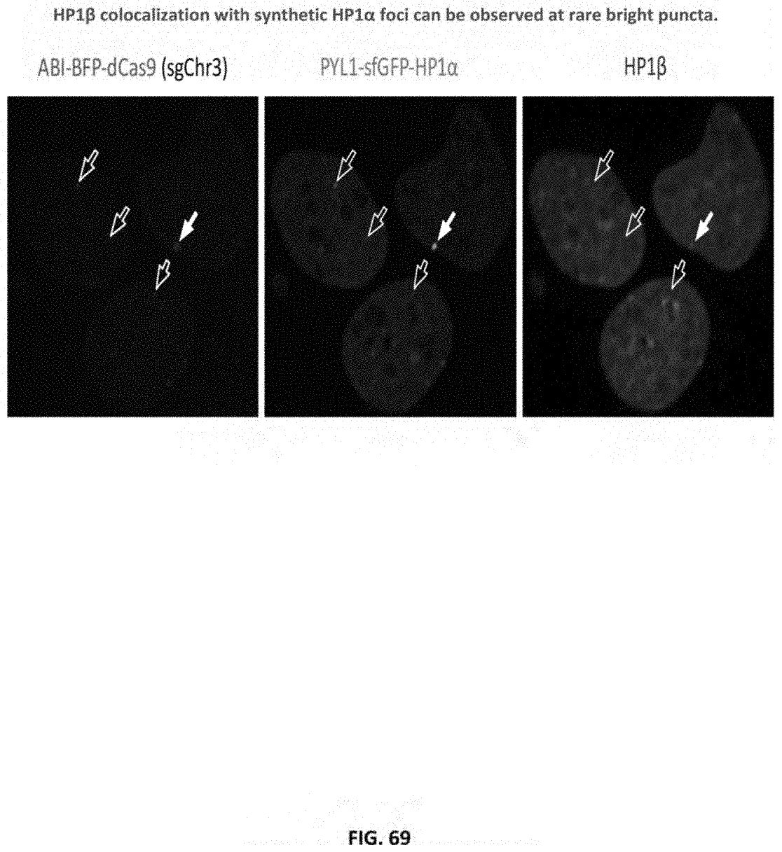

HP1β is an ortholog of HP1α and also was enriched within natural heterochromatin. The enrichment of HP1β was tested by immunofluorescence and fluorescence tracing. Cells were immunostained with HP1β primary and AlexaFluor647 secondary antibodies after 2 days of 100 μM ABA treatment. FIG. 69 show that HP1β colocalization with synthetic HP1α foci was observed at rare bright puncta. Cells that were immunostained with HP1β (right panel) localized to the synthetic heterochromatin foci formed by PYL1-sfGFP-HP1α (middle panel) and ABI-BFP-dCas9 (left panel) at bright puncta (solid arrow) but not other puncta (hollow arrows), after 2 days ABA treatment. While most synthetic heterochromatin foci did not show notable HP1β enrichment, rare bright foci did. This result suggests that synthetic heterochromatin foci were recruiting HP1β, but in most cases not in sufficient quantities to discern via imaging.

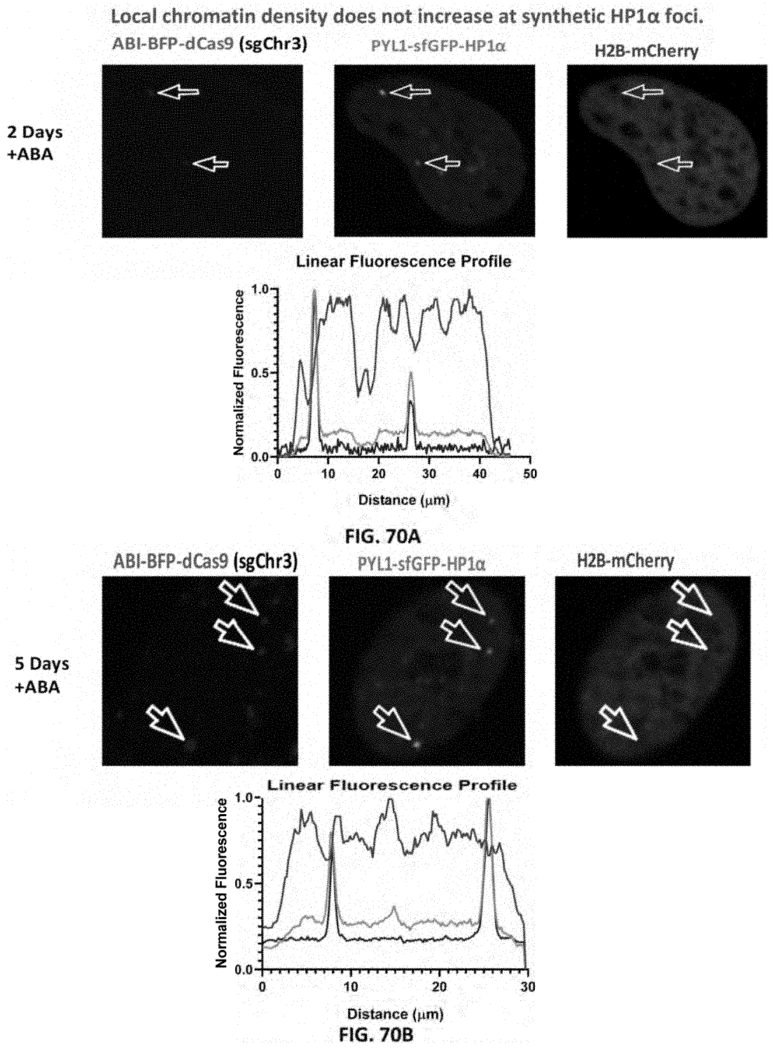

Local chromatin density does not increase at synthetic HP1α foci. FIG. 70A and FIG. 70B show representative fluorescence images taken 2 days and 5 days after ABA treatment respectively, representing histone density as measured by mCherry-H2B fluorescence signal (third panel) at the synthetic heterochromatin foci (arrows) formed by PYL1-sfGFP-HP1α (second panel) and ABI-BFP-dCas9 (first panel). Corresponding normalized linear fluorescence profile of the co-localization of the mCherry-H2B with synthetic heterochromatin puncta at peaks of PYL1-sfGFP-HP1α and ABI-BFP-dCas9 (right panel) was measured by selective enrichment of mCherry-H2B. The fluorescence intensity of H2B-mCherry was used as a proxy measurement of histone and therefore chromatin density within different regions of the nucleus. H2B-mCherry signal did not appear to be selectively enriched at synthetic heterochromatin sites after either 2 or 5 days of 100 μM ABA treatment. Line and puncta used for linear tracing were used to confirm these findings.

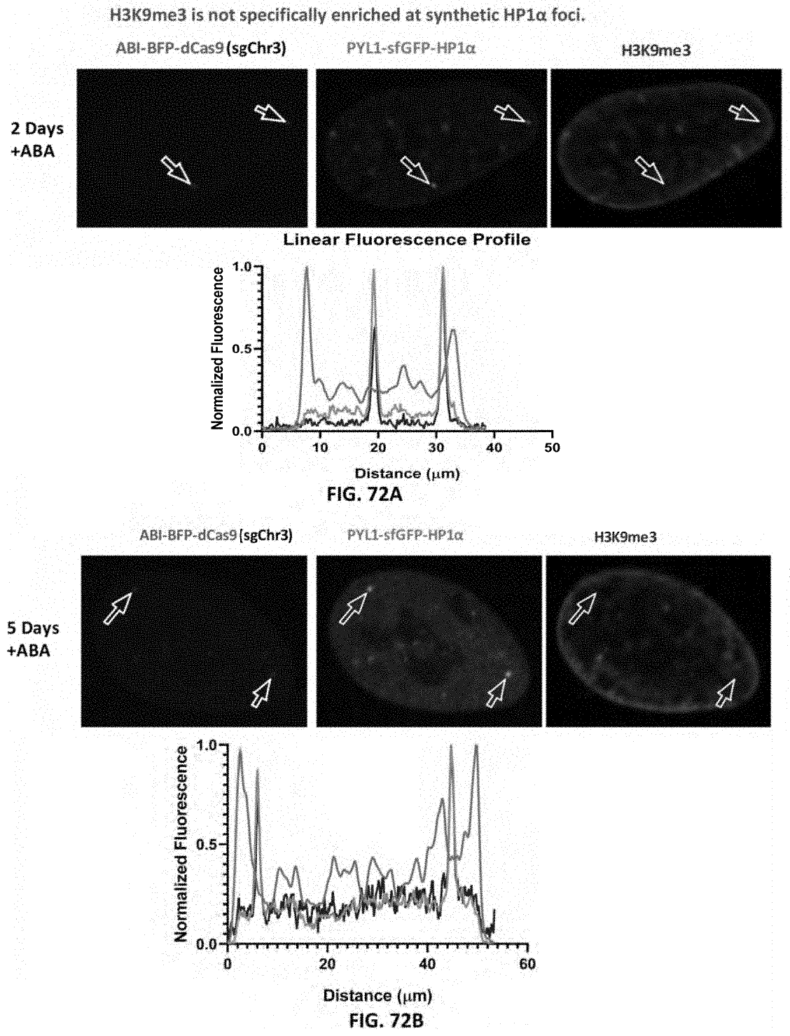

Another key marker of HP1α-associated natural heterochromatin is the post-translational trimethylation of lysine 9 on the tail of histone 3 (H3K9me3). Cells were immunostained with H3K9me3 primary and AlexaFluor647 secondary antibodies after 2 or 5 days of 100 uM ABA treatment. FIG. 72A and FIG. 72B show representative fluorescence images taken 2 days and 5 days after ABA treatment respectively. The synthetic heterochromatin foci formed by PYL1-sfGFP-HP1α (second panel) and ABI-BFP-dCas9 (first panel) are represented by arrow, and immunostaining with H3K9me3 is shown in the third panel. Corresponding normalized linear fluorescence profile of the co-localization of the H3K9me3 with synthetic heterochromatin puncta showing no selective enrichment of H3K9me3 at peaks of PYL1-sfGFP-HP1α and ABI-BFP-dCas9 is shown by linear tracing. For the both graphs, the line with the first peak is the H3K9me3 staining, and lines that are overlapping with two peaks are the PYL1-sfGFP-HP1α and ABI-BFP-dCas9.

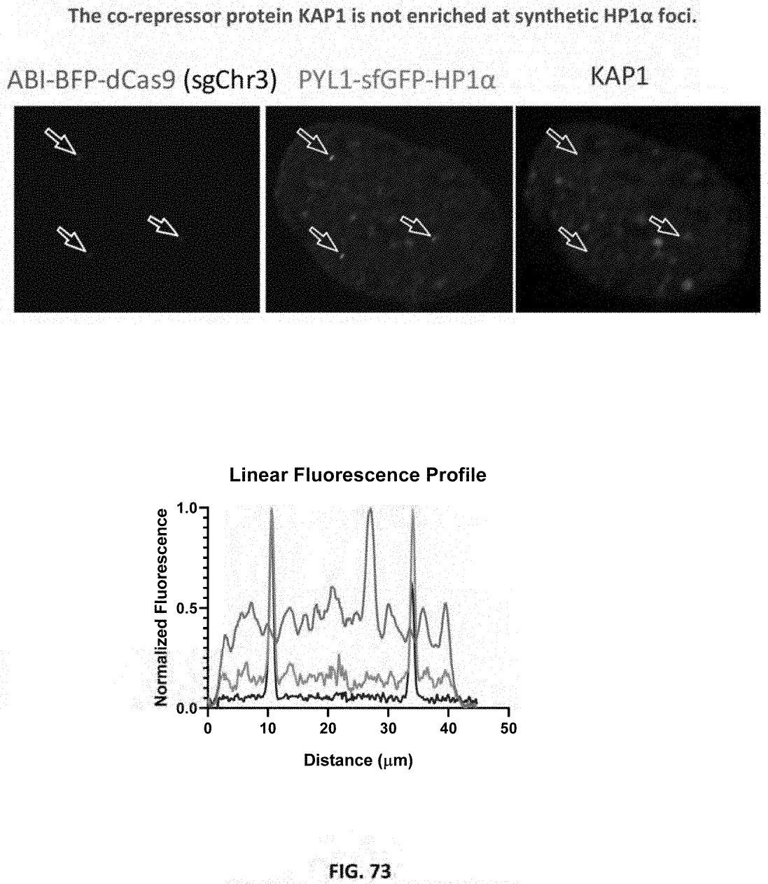

The presence of KAP1, a co-repressor scaffold protein that binds both HP1α and other chromatin modifying proteins, at synthetic HP1α foci was also assayed using immunostaining. Cells were immunostained with KAP1 primary and AlexaFluor647 secondary antibodies after 2 days of 100 μM ABA treatment FIG. 73 shows that the co-repressor protein KAP1 is not enriched at synthetic HP1α foci. Representative fluorescence images taken 2 days after ABA treatment, depicting cells immunostained with KAP1 (third panel) and synthetic heterochromatin foci (arrows) formed by PYL1-sfGFP-HP1α (second panel) and ABI-BFP-dCas9 (first panel) are shown. Corresponding normalized linear fluorescence profile of the co-localization of the KAP1 with synthetic heterochromatin puncta was shown no selective enrichment of KAP1 at peaks of PYL1-sfGFP-HP1α and ABI-BFP-dCas9 using linear tracing. For the graph, the dark higher line is the KAP1 staining, and lines that are overlapping with two peaks are the PYL1-sfGFP-HP1α and ABI-BFP-dCas9. KAP1 was not selectively enriched at synthetic HP1α foci despite co-localization with PYL1-sfGFP-HP1α at natural heterochromatin sites.

### Example 34: Mutational Analysis of HP1α for Synthetic Heterochromatin Generation

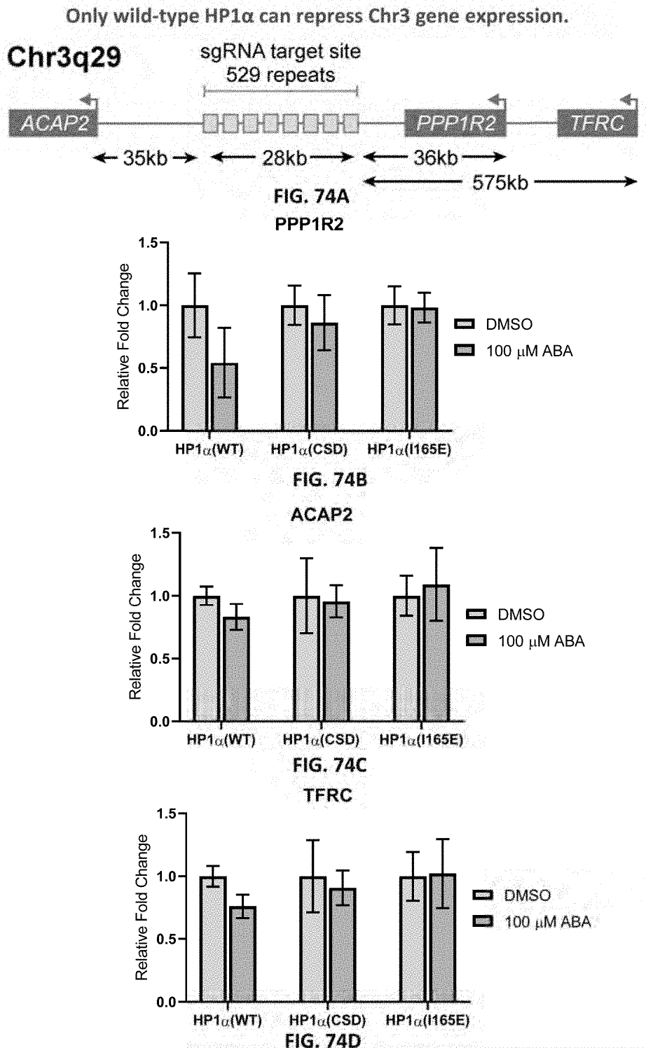

To determine whether the repression resulting from HP1α is due to the function of any particular domain within HP1α, mutant HP1α in which the chromodomain is removed, e.g. HP1α (CSD), and in which the chromoshadow domain is rendered incapable of homodimerizing, e.g. HP1α (I165E), were tested against full length wild-type HP1α for repression on Chr3q29. Only full length wild-type HP1α was capable of repressing the three tested distal genes as measured by RT-qPCR. FIG. 74A shows the schematic illustration of the CRISPR-GO system by recruitment of PYL1-sfGFP-HP1α to sgRNA target sites at the Chr3:q29 locus in U2OS cells. ACAP2 is located ˜35 kb upstream of the sgRNA target site, PPP1R2 is located ˜36 kb downstream of the sgRNA target site, and TFRC is located about 575 kb downstream of the sgRNA target site. FIG. 74B shows the graph of comparison of PPP1R2 gene expression (measured by RT-qPCR) in U2OS cells expressing wild-type HP1α (HP1α-WT), mutant HP1α (CSD) or mutant HP1α (I165E) with PYL1-sfGFP-HP1α to the Chr3:q29 loci in +/− ABA conditions. Only wild-type HP1α was able to repress PPP1R2 gene expression. FIG. 74C shows the graph of comparison of ACAP2 gene expression (measured by RT-qPCR) in U2OS cells expressing wild-type HP1α, mutant HP1α (CSD) or mutant HP1α (I165E) with PYL1-sfGFP-HP1α to the Chr3:q29 loci in +/− ABA conditions. Only wild-type HP1α was able to repress ACAP2 gene expression. FIG. 74D shows the graph of comparison of TFRC gene expression (measured by RT-qPCR) in U2OS cells expressing wild-type HP1α, mutant HP1α (CSD) or mutant HP1α (I165E) with PYL1-sfGFP-HP1α to the Chr3:q29 loci in +/− ABA conditions. Only wild-type HP1α was able to repress TFRC gene expression. Cells transfected with a non-targeting sgRNA (sgNT) were used as control. Data are represented as mean±SD. HP1α(CSD) has been reported to be capable of repressing proximal genes and locally depositing H3K9me3 marks. These results suggest that the repression observed on Chr3q29 by full-length HP1α utilize a distinct mechanism from the previously reported HP1α(CSD) mechanism.

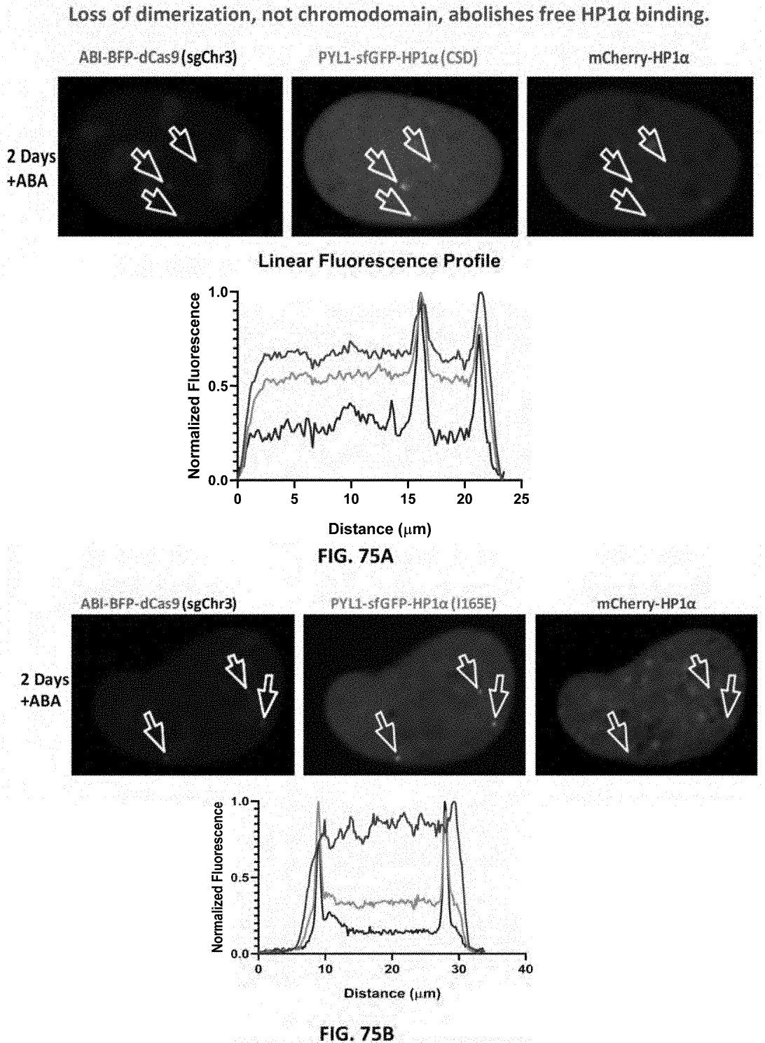

To understand how the heterochromatin properties of the HP1α(I165E) and HP1α(CSD) mutants differ from the full length wild-type protein, their abilities to recruit free-floating mCherry-HP1α were examined. While the HP1α(CSD) was still capable of recruiting mCherry-HP1α to synthetic HP1α foci, the HP1α(I165E) mutant lost this capacity. FIG. 75 shows that loss of dimerization, but not the chromodomain, abolishes free HP1α at synthetic HP1α foci (arrows). FIG. 75A shows representative fluorescence images taken 2 days after ABA treatment, showing recruitment of free-floating mCherry-HP1α (third panel) to the synthetic heterochromatin foci (arrows) formed by PYL1-sfGFP-HP1α (CSD) (second panel) and ABI-BFP-dCas9 (first panel). Line and puncta linear tracing was used to confirm the co-localization analysis shown in the corresponding normalized linear fluorescence profile of the co-localization of the mCherry-HP1α with synthetic heterochromatin puncta as seen by selective enrichment of mCherry-HP1α at peaks of PYL1-sfGFP-HP1α and ABI-BFP-dCas9 (graph). FIG. 75B shows representative fluorescence images taken 2 days after ABA treatment, representing no recruitment of free-floating mCherry-HP1α (third panel) to the synthetic heterochromatin foci (arrows) formed by PYL1-sfGFP-HP1α (I165E) (second panel) and ABI-BFP-dCas9 (first panel nd corresponding normalized linear fluorescence profile of the co-localization of the mCherry-HP1α with synthetic heterochromatin puncta showing no selective enrichment of mCherry-HP1α at peaks of PYL1-sfGFP-HP1α and ABI-BFP-dCas9 (graph). This suggested that the mechanism by which HP1α enacts distal gene repression relies on its homodimerization and/or subsequent interaction with PxVxL motif transcription factors at the dimerized chromoshadow interface.

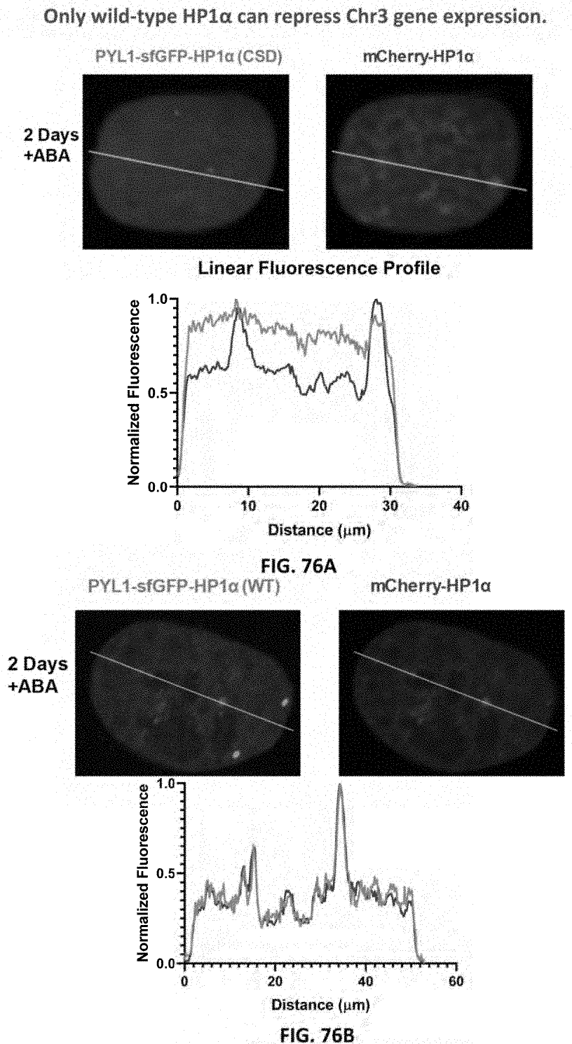

While the HP1α(CSD) mutant was still competent for recruiting free-floating HP1α to synthetic HP1α foci, the removal of the N-terminus and the chromodomain impairs its ability to localize to natural heterochromatin sites (using mCherry-HP1α subnuclear localization as a proxy). FIG. 76 shows that loss of chromodomain impairs normal HP1α nuclear distribution. FIG. 76A shows representative fluorescence images taken 2 days after ABA treatment, representing normal HP1α nuclear distribution as seen by mCherry-HP1α fluorescence (middle panel) impaired HP1α nuclear distribution as seen by PYL1-sfGFP-HP1α (CSD) (left panel), and the corresponding normalized linear fluorescence profile of the mCherry-HP1α and PYL1-sfGFP-HP1α (CSD) signals (graph) showing reduced dynamic range for PYL1-sfGFP-HP1α (CSD). FIG. 76B shows representative fluorescence images taken 2 days after ABA treatment, representing normal HP1α nuclear distribution as seen by mCherry-HP1α fluorescence (middle panel), normal HP1α nuclear distribution as seen by PYL1-sfGFP-HP1α (WT) fluorescence (left panel), and the corresponding normalized linear fluorescence profile of the mCherry-HP1α and PYL1-sfGFP-HP1α (WT) signals (graph) showing equivalent dynamic range for PYL1-sfGFP-HP1α (WT). These results suggest that intact N-terminus and/or chromodomain are important for the distal gene repression effect by full length HP1α.

### Example 35: KRAB as an Alternative Approach for Heterochromatin Formation

To determine whether the incomplete heterochromatin marks from synthetic HP1α was due to impaired HP1α function as a result of fusing additional protein domains to its N-terminus, the ability for indirectly recruiting unencumbered, endogenous HP1α was tested. In this alternative strategy, HP1α in the inducible system was replaced with the KRAB domain commonly used for CRISPR interference (CRISPRi). KRAB acts as a recruitment signal for the co-repressor scaffold protein KAP1, which in turn recruits endogenous HP1α (Ecco et. al., Development 2017).

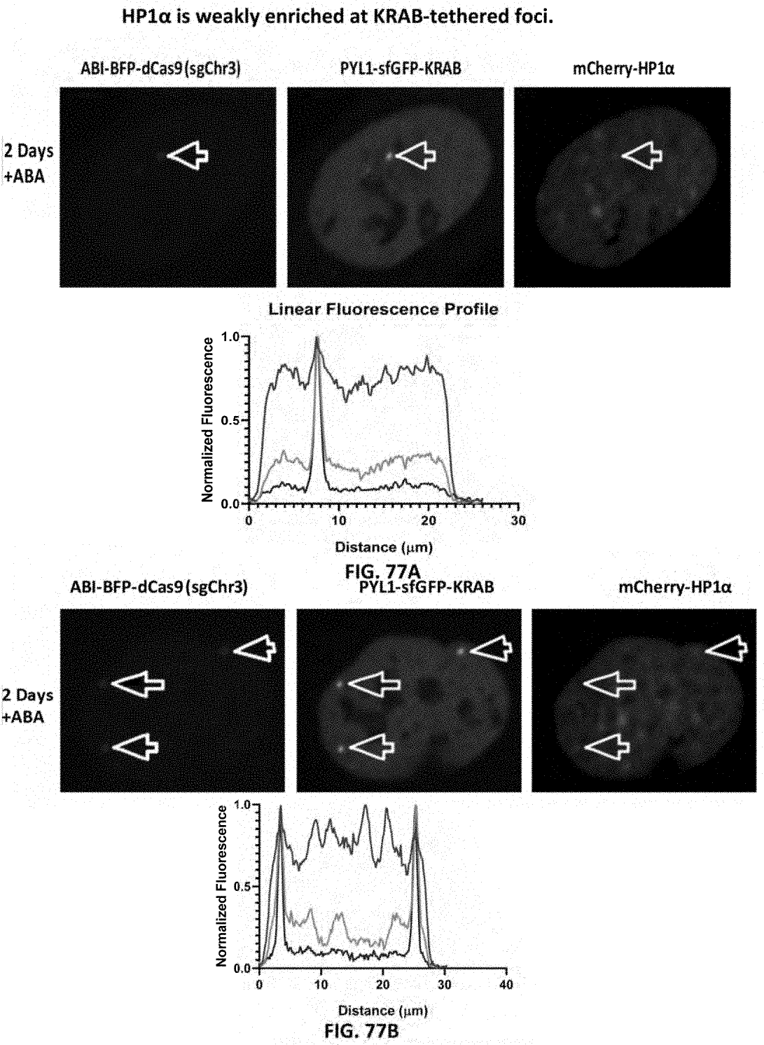

The alternative KRAB heterochromatin system also demonstrated the ability to recruit free-floating HP1α to synthetic heterochromatin foci, though at seemingly lower levels than for the original synthetic HP1α system. Immunofluorescence analysis showed that HP1α localization is weakly enriched at KRAB-tethered foci. FIG. 77 shows that HP1α localization is weakly enriched at KRAB-tethered foci. FIG. 77A shows representative fluorescence images taken 2 days after ABA treatment, showing recruitment of free-floating mCherry-HP1α (third panel, top) to the synthetic heterochromatin foci (arrows) shown by PYL1-sfGFP-KRAB (second panel, top) and ABI-BFP-dCas9 (first panel, top). Line and puncta used for linear trace was used in the corresponding normalized linear fluorescence profile of the co-localization of the mCherry-HP1α with synthetic heterochromatin puncta, which showed selective enrichment of mCherry-HP1α at peaks of PYL1-sfGFP-KRAB and ABI-BFP-dCas9. FIG. 77B shows representative fluorescence images taken 2 days after ABA treatment, showing recruitment of free-floating mCherry-HP1α (third panel, bottom) to the synthetic heterochromatin foci (arrows) shown by PYL1-sfGFP-KRAB (second panel, bottom) and ABI-BFP-dCas9 (first panel, bottom). Line and puncta used for linear trace was used in the corresponding normalized linear fluorescence profile of the co-localization of the mCherry-HP1α with synthetic heterochromatin puncta, which showed selective enrichment of mCherry-HP1α at peaks of PYL1-sfGFP-KRAB and ABI-BFP-dCas9 (graph).

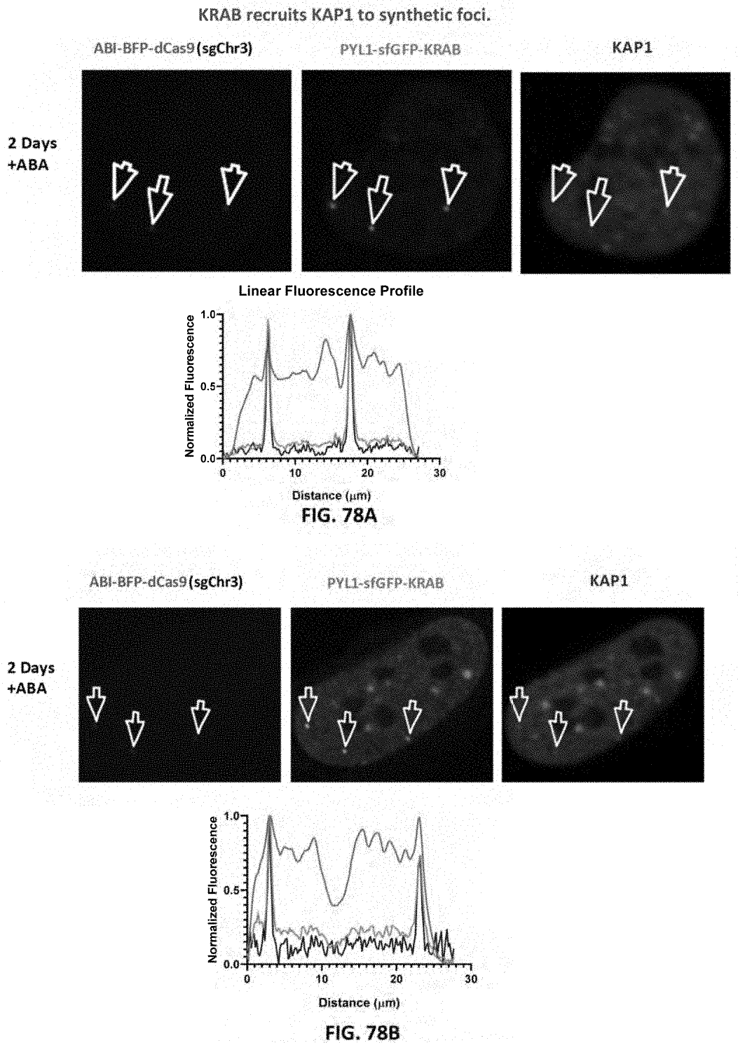

Unlike with the original synthetic HP1α system, the KRAB system was enriched for KAP1 at the synthetic heterochromatin foci. FIG. 78 shows that KRAB recruits KAP1 to synthetic foci. Cells were immunostained with KAP1 primary and AlexaFluor647 secondary antibodies after 2 days of 100 μM ABA treatment. FIG. 78A shows representative fluorescence images taken 2 days after ABA treatment, representing cells immunostained for KAP1 (third panel, top) and synthetic heterochromatin foci (arrows) shown by PYL1-sfGFP-KRAB (middle panel, top) and ABI-BFP-dCas9 (left panel, top). Line and puncta tracing was used for the corresponding normalized linear fluorescence profile of the co-localization of KAP1 with synthetic heterochromatin puncta, which showed selective enrichment of KAP1 at peaks of PYL1-sfGFP-KRAB and ABI-BFP-dCas9 (top graph). FIG. 78B shows representative fluorescence images taken 2 days after ABA treatment, showing cells immunostained with KAP1 (third panel, bottom) and the synthetic heterochromatin foci (arrows) shown by PYL1-sfGFP-KRAB (second panel, bottom) and ABI-BFP-dCas9 (first panel, bottom). Line and puncta tracing was used in the corresponding normalized linear fluorescence profile of the co-localization of the KAP1 with synthetic heterochromatin puncta, which showed selective enrichment of KAP1 at peaks of PYL1-sfGFP-KRAB and ABI-BFP-dCas9 (graph, bottom).

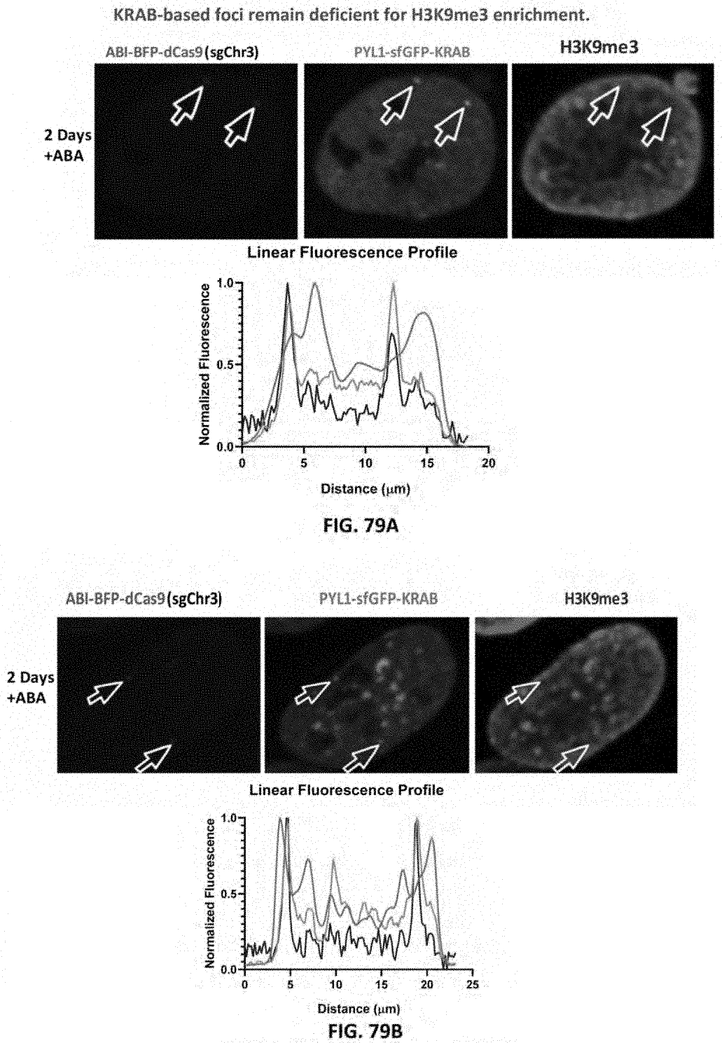

Despite recruiting both HP1α and KAP1 to synthetic foci, KRAB was still unable to increase H3K9me3 abundance at the Chr3q29 target site. FIG. 79 shows that KRAB-based foci remain deficient for H3K9me3 enrichment. Cells were immunostained with KAP1 primary and AlexaFluor647 secondary antibodies after 2 days of 100 μM ABA treatment. FIG. 79A shows representative fluorescence images taken 2 days after ABA treatment, showing cells immunostained for H3K9me3 (third panel, top), synthetic heterochromatin foci (arrows) shown by PYL1-sfGFP-KRAB (second panel, top) and ABI-BFP-dCas9 (right panel, top). Line and puncta tracing was used for corresponding normalized linear fluorescence profile of the co-localization of H3K9me3 with synthetic heterochromatin puncta, which showed no selective enrichment of H3K9me3 at peaks of PYL1-sfGFP-KRAB and ABI-BFP-dCas9 (graph, top). FIG. 79B shows representative fluorescence images taken 2 days after ABA treatment, representing cells immunostained with H3K9me3 (third panel, bottom) and synthetic heterochromatin foci (arrows) shown by PYL1-sfGFP-KRAB (second panel, bottom) and ABI-BFP-dCas9 (left panel, bottom). Line and puncta tracing was used for the corresponding normalized linear fluorescence profile of the co-localization of the H3K9me3 with synthetic heterochromatin puncta, which showed no selective enrichment of H3K9me3 at peaks of PYL1-sfGFP-KRAB and ABI-BFP-dCas9 (graph, bottom).

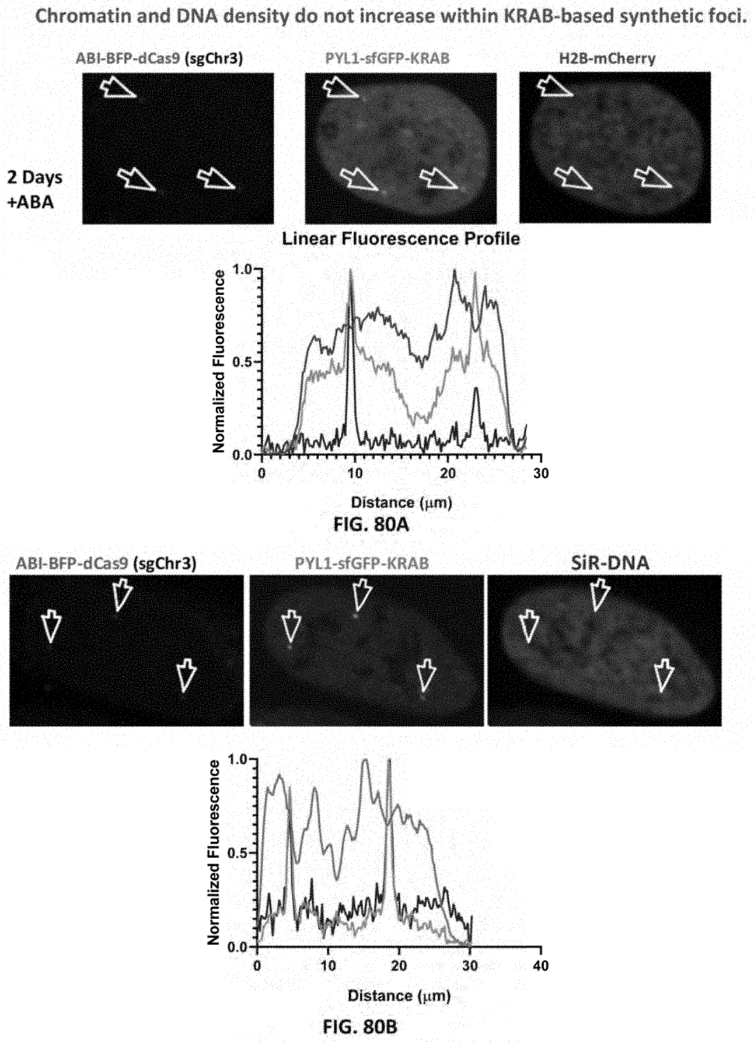

Similarly, local chromatin/histone density, as measured by H2B-mCherry fluorescence, nor DNA density, as measured by SiR-DNA staining intensity, was increased at KRAB-based synthetic foci. FIG. 80A shows representative fluorescence images taken 2 days after ABA treatment, showing histone density as measured by mCherry-H2B (third panel, top) fluorescence signal at the synthetic heterochromatin foci (arrows) shown by PYL1-sfGFP-KRAB (second panel, top) and ABI-BFP-dCas9 (first panel, top). Line and puncta tracing was used for the corresponding normalized linear fluorescence profile of the mCherry-H2B signal at synthetic heterochromatin puncta, which showed no selective enrichment of mCherry-H2B at peaks of PYL1-sfGFP-KRAB and ABI-BFP-dCas9 (graph, top). FIG. 80B shows representative fluorescence images taken 2 days after ABA treatment, representing DNA density as measured by SiR-DNA staining (third panel, bottom) at the synthetic heterochromatin foci (arrows) as shown by PYL1-sfGFP-KRAB (second panel, bottom) and ABI-BFP-dCas9 (left panel, bottom). Line and puncta tracing was used for the corresponding normalized linear fluorescence profile of the siR-DNA signal at synthetic heterochromatin puncta, which showed no selective enrichment of siR-DNA at peaks of PYL1-sfGFP-KRAB and ABI-BFP-dCas9 (graph, bottom).

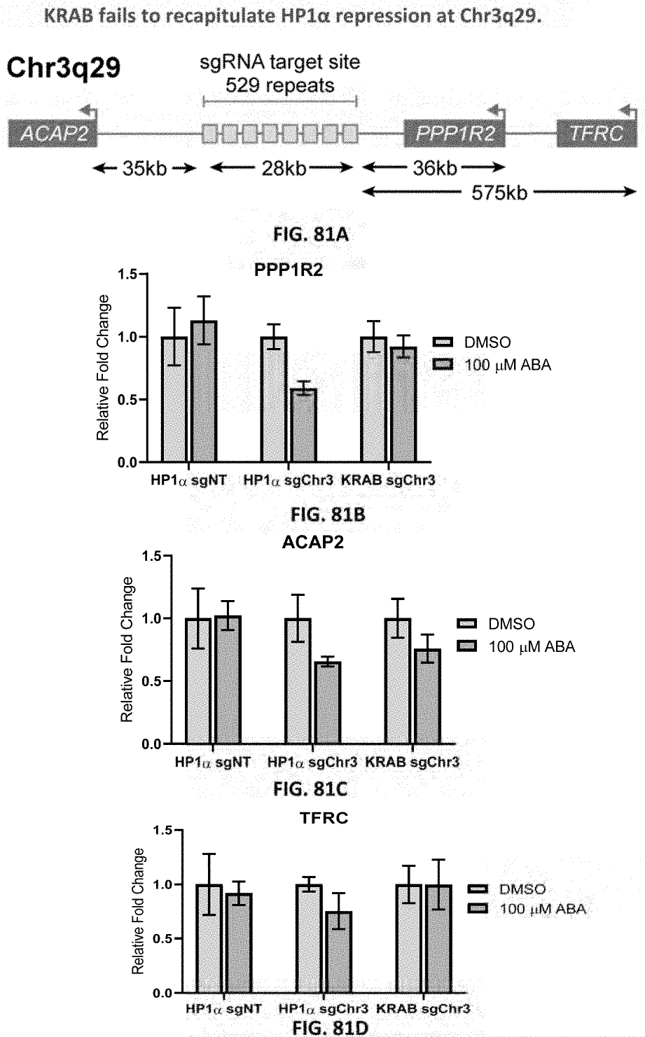

FIG. 81 show that KRAB fails to recapitulate HP1α repression at Chr3q29. RT-qPCR after 5 days of 100 μM ABA treatment showed that while KRAB repressed ACAP2, PPP1R2 and TFRC remained unaffected by its recruitment to the Chr3q29 tandem repeat target sites. FIG. 81A shows the schematic illustration of the CRISPR-GO system by recruitment of PYL1-sfGFP-HP1α or PYL1-sfGFP-KRAB to sgRNA target sites at the Chr3:q29 locus in U2OS cells. ACAP2 is located ˜35 kb upstream of the sgRNA target site, PPP1R2 is located ˜36 kb downstream of the sgRNA target site and TFRC is located about 575 kb downstream of the sgRNA target site. FIG. 81B shows the graph of comparison of PPP1R2 gene expression (measured by RT-qPCR) in U2OS cells expressing PYL1-sfGFP-HP1α or PYL1-sfGFP-KRAB to the Chr3:q29 loci in +/− ABA conditions. FIG. 81C shows the graph of comparison of ACAP2 gene expression (measured by RT-qPCR) in U2OS cells expressing PYL1-sfGFP-HP1α or PYL1-sfGFP-KRAB to the Chr3:q29 loci in +/− ABA conditions. FIG. 81D shows the graph of comparison of TFRC gene expression (measured by RT-qPCR) in U2OS cells expressing PYL1-sfGFP-HP1α or PYL1-sfGFP-KRAB to the Chr3:q29 loci in +/− ABA conditions. Cells transfected with a non-targeting sgRNA (sgNT) were used as control. Data are represented as mean±SD.

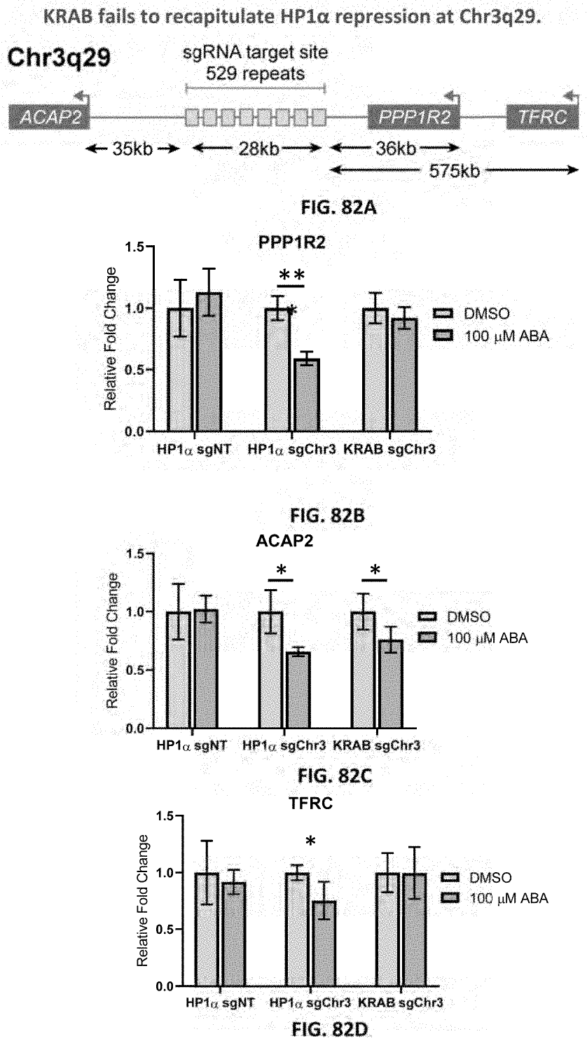

The KRAB and HP1α systems were also tested at an alternative genomic context on Chr1p36. This target site contains 36 copies of a tandem repeat region, with closer proximity to flanking genes. The transcriptional start site of the nearest gene, DVL1, lies 0.9 kb away from the tandem repeat site. In this site, RT-qPCR after 5 days ABA treatment showed KRAB was able to strongly repress DVL1 expression but exerted no notable repression of distal genes INTS11 and CPTP. On the other hand, HP1α completely failed to repress any of the three genes. FIG. 82 shows that KRAB but not HP1α represses proximally at Chr1p36 repeat. FIG. 82A shows the schematic illustration of the CRISPR-GO system by recruitment of PYL1-sfGFP-HP1α or PYL1-sfGFP-KRAB to sgRNA target sites at the Chr1:p36 locus in U2OS cells. CPTP is located ˜25 kb upstream of the sgRNA target site, INTS11 is located ˜26 kb upstream of the sgRNA target site, and DVL1 is located about 0.9 kb upstream of the sgRNA target site. FIG. 82B shows the graph of comparison of DVL1 gene expression (measured by RT-qPCR) in U2OS cells expressing PYL1-sfGFP-HP1α or PYL1-sfGFP-KRAB to the Chr1:p36 loci in +/−ABA conditions. FIG. 82C shows the graph of comparison of CPTP gene expression (measured by RT-qPCR) in U2OS cells expressing PYL1-sfGFP-HP1α or PYL1-sfGFP-KRAB to the Chr1:p36 loci in +/− ABA conditions. FIG. 82D shows the graph of comparison of INTS11 gene expression (measured by RT-qPCR) in U2OS cells expressing PYL1-sfGFP-HP1α or PYL1-sfGFP-KRAB to the Chr1:p36 loci in +/− ABA conditions. Cells transfected with a non-targeting sgRNA (sgNT) were used as control. Data are represented as mean±SD. This suggested that HP1α and KRAB tethering appeared to repress via distinct primary mechanisms.

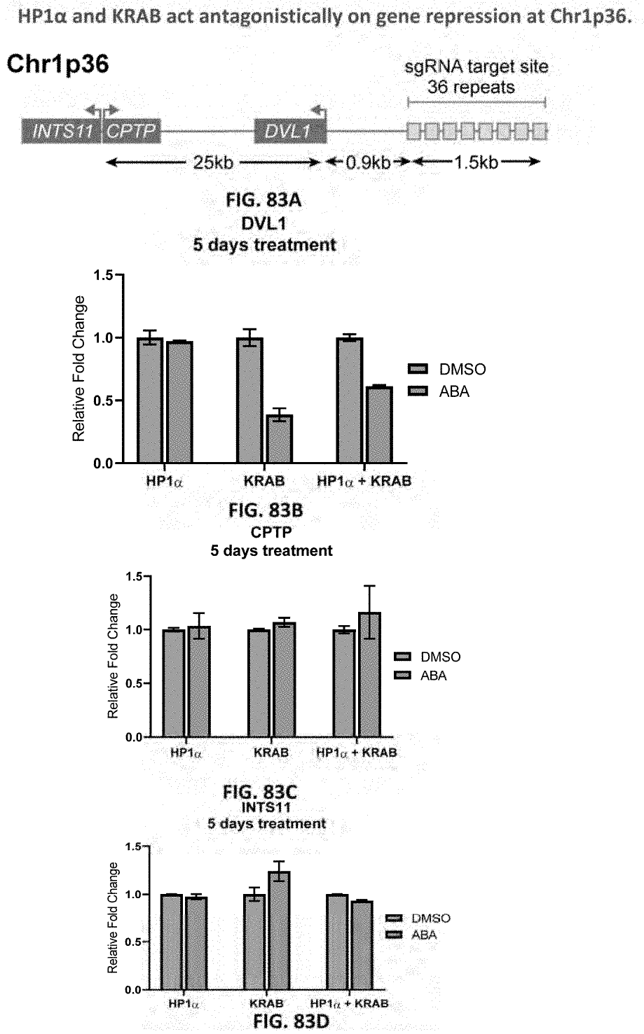

Gene expression at the Chr1p36 target site was affected when both KRAB and HP1α were recruited to the same site. FIG. 83 shows that HP1α and KRAB act antagonistically on gene repression at Chr1p36. For this experiment, U2OS cells expressing ABI-BFP-dCas9 and sgChr1p36, co-expressing PYL1-sfGFP-HP1α and PYL1-mCherry-KRAB alone or in combination, were treated with 100 μM ABA for 5 days, after which gene expression at Chr1p36 is quantified by RT-qPCR (sgChr1p36: GCGACGGGGGGAGTGAGGAG (SEQ ID NO: 38); target PAM sequence: GGG (SEQ ID NO: 39)). In further support that KRAB and HP1α repress via distinct mechanisms, when both KRAB and HP1α are recruited to the Chr1p36 tandem repeat site, the repressive effect of KRAB on DVL1 is diluted out, likely due to the fact that half of the tandem repeat sites are occupied by inert HP1α rather than KRAB. FIG. 83A shows the schematic illustration of the recruitment of PYL1-sfGFP-HP1α, PYL1-sfGFP-KRAB or both to sgRNA target sites at the Chr1:p36 locus in U2OS cells. CPTP is located ˜25 kb upstream of the sgRNA target site, INTS11 is located ˜26 kb upstream of the sgRNA target site and DVL1 is located about 0.9 kb upstream of the sgRNA target site. FIG. 83B shows the graph of comparison of DVL1 gene expression (measured by RT-qPCR) in U2OS cells expressing PYL1-sfGFP-HP1α, PYL1-sfGFP-KRAB or both to the Chr1:p36 locus in +/− ABA conditions. FIG. 83C shows the graph of comparison of CPTP gene expression (measured by RT-qPCR) in U2OS cells expressing PYL1-sfGFP-HP1α, PYL1-sfGFP-KRAB or both to the Chr1:p36 locus in +/− ABA conditions. FIG. 83D shows the graph of comparison of gene expression of INTS11 (measured by RT-qPCR) in U2OS cells expressing PYL1-sfGFP-HP1α, PYL1-sfGFP-KRAB or both to the Chr1:p36 locus in +/− ABA conditions. Data are represented as mean±SD.

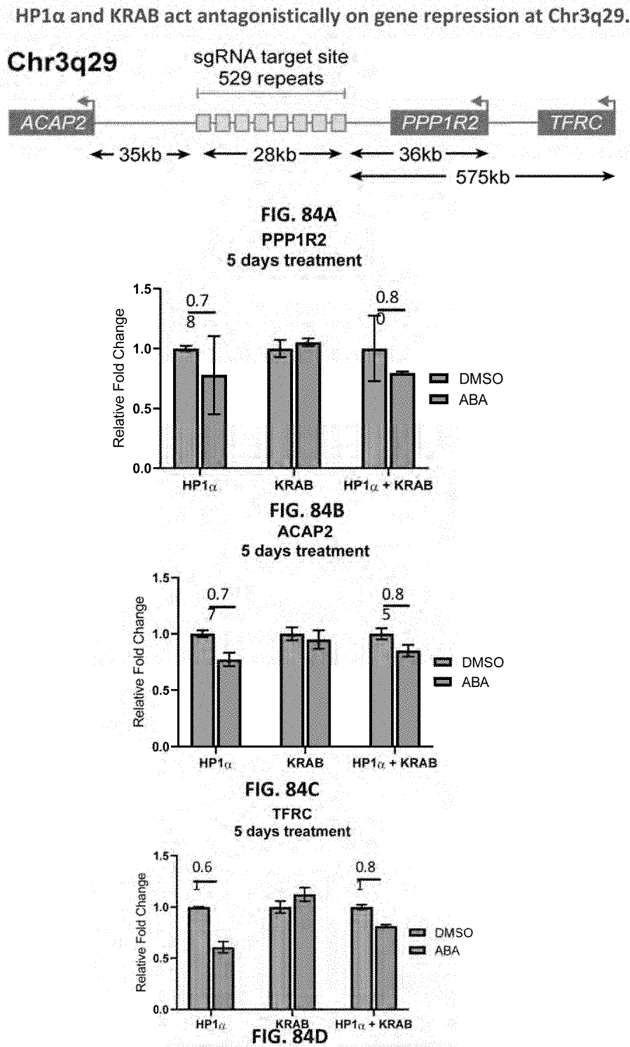

Likewise, when the same experiment was performed for the Chr3q29 test site, the distal gene repression effect observed for HP1α alone was diluted out when KRAB was co-recruited the tandem repeat site. FIG. 84 shows that HP1α and KRAB act antagonistically on gene repression at Chr3q29. FIG. 84A shows the schematic illustration of the CRISPR-GO system by recruitment of PYL1-sfGFP-HP1α, PYL1-sfGFP-KRAB or both to sgRNA target sites at the Chr3:q29 locus in U2OS cells. ACAP2 is located ˜35 kb upstream of the sgRNA target site, PPP1R2 is located ˜36 kb downstream of the sgRNA target site and TFRC is located about 575 kb downstream of the sgRNA target site. FIG. 84B shows the graph of comparison of PPP1R2 gene expression (measured by RT-qPCR) in U2OS cells expressing PYL1-sfGFP-HP1α, PYL1-sfGFP-KRAB or both to the Chr1:p36 locus in +/− ABA conditions. Only cells expressing PYL1-sfGFP-KRAB or both to the Chr1:p36 locus showed repression under ABA conditions. FIG. 84C shows the graph of comparison of ACAP2 gene expression (measured by RT-qPCR) in U2OS cells expressing PYL1-sfGFP-HP1α, PYL1-sfGFP-KRAB or both to the Chr1:p36 locus in +/− ABA conditions. FIG. 84D shows the graph of comparison of gene expression of TFRC (measured by RT-qPCR) in U2OS cells expressing PYL1-sfGFP-HP1α, PYL1-sfGFP-KRAB or both to the Chr1:p36 locus in +/− ABA conditions. Data are represented as mean±SD.

### Example 36: Direct Writing of H3K9me3 Marks by Histone Methyltransferases

To better understand why H3K9me3 enrichment was not observed with either synthetic HP1α or KRAB-based heterochromatin, the effector in the inducible system was replaced with SUV39H1, a human histone methyltransferase that enzymatically deposits H3K9me3 marks. If the locus was amenable to H3K9me3 and confocal microscopy had sufficient resolving power, enrichment of H3K9me3 would be expected to be observed when SUV39H1 is recruited to the Chr3q29 site.

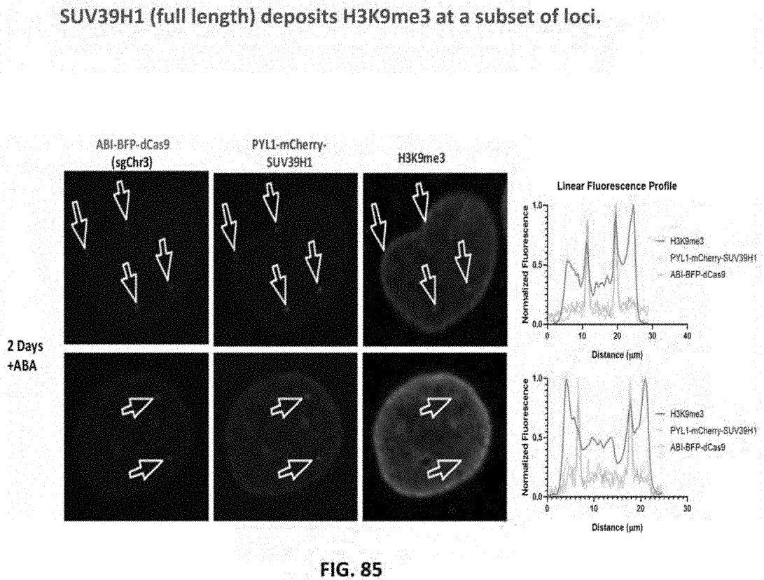

FIG. 85 shows that H3K9me3 is deposited at a subset of foci by SUV39H1 (full length). After two days of ABA treatment, U2OS cells in which SUV39H1 was recruited to the Chr3q29 site showed a subset of synthetic puncta in which H3K9me3 was selectively enriched. FIG. 85 shows representative fluorescence images taken 2 days after ABA treatment, depicting cells immunostained with H3K9me3 (third panel, top and bottom), to the synthetic heterochromatin foci formed by PYL1-mCherry-SUV39H1 (second panel, top and bottom) and ABI-BFP-dCas9 (left panel, top and bottom). These results confirm that the locus being tested was indeed capable of accepting H3K9me3 modifications and such changes can be observed via microscopy. Line and puncta tracing was used for corresponding normalized linear fluorescence profiles for the co-localization of the H3K9me3 with synthetic heterochromatin puncta, which showed selective enrichment of H3K9me3 at peaks of PYL1-sfGFP-KRAB and ABI-BFP-dCas9 (graphs, top and bottom). These results confirm that the locus being tested was indeed capable of accepting H3K9me3 modifications and that such changes could be observed via microscopy.

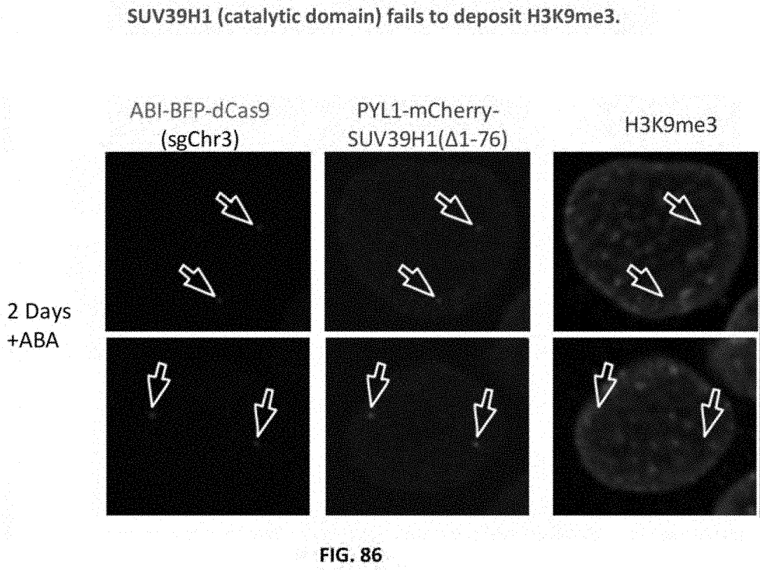

Additional histone methyltransferase variants were also tested. Unlike the full length SUV39H1, the catalytic domain variant of SUV3H1, in which the first 76 amino acids of the protein (containing the HP1-interacting domain and the chromodomain) was removed and failed to deposit H3K9me3 marks at the Chr3q29 target site. FIG. 86 shows that H3K9me3 is not deposited at foci by SUV39H1(Δ1-76). FIG. 86 shows that H3K9me3 was deposited at a subset of foci by SUV39H1 (Δ1-76). FIG. 86 shows representative fluorescence images taken 2 days after ABA treatment, depicting cells immunostained with H3K9me3 (right panel, top and bottom) and synthetic heterochromatin foci formed by PYL1-mCherry-SUV39H1 (41-76) (middle panel, top and bottom) and ABI-BFP-dCas9 (left panel, top and bottom). This result suggested that the methyltransferase activity of SUV39H1 was dependent on its interaction with HP1 family proteins.

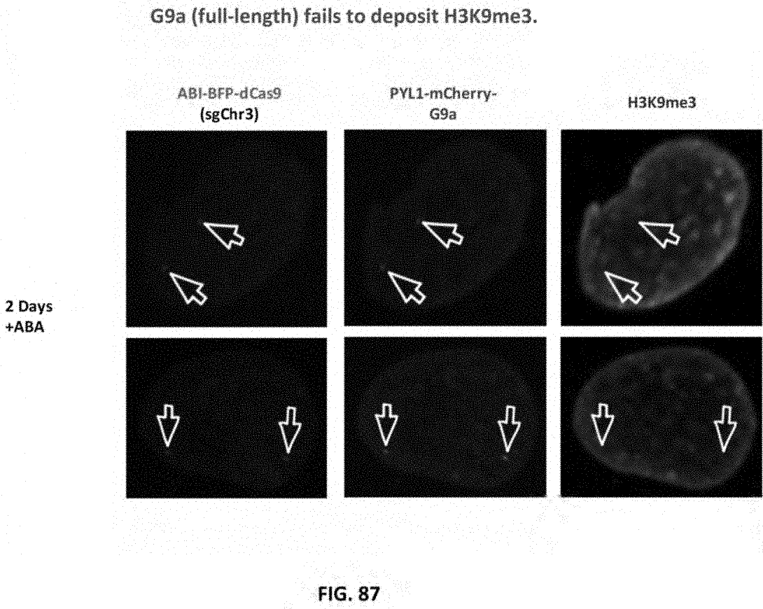

A second human histone methyltransferase, G9a was also tested. While G9a directly catalyzes H3K9me1 and H3K9me2 deposition, its presence at a locus is associated with increased H3K9me3, possibly via indirect mechanisms such as generation of H3K9me2 substrates to facilitate further modification to H3K9me3 or through complex formation with SUV39H1. No enrichment of H3K9me3 was observed when full-length G9a was fused to the inducible system and recruited to Chr3q29. FIG. 87 shows that H3K9me3 is not deposited at G9a (full length). FIG. 87 shows representative fluorescence images taken 2 days after ABA treatment, depicting cells immunostained with H3K9me3 (right panel, top and bottom) and synthetic heterochromatin foci formed by PYL1-mCherry-G9a (full length) (middle panel, top and bottom) and ABI-BFP-dCas9 (left panel, top and bottom).

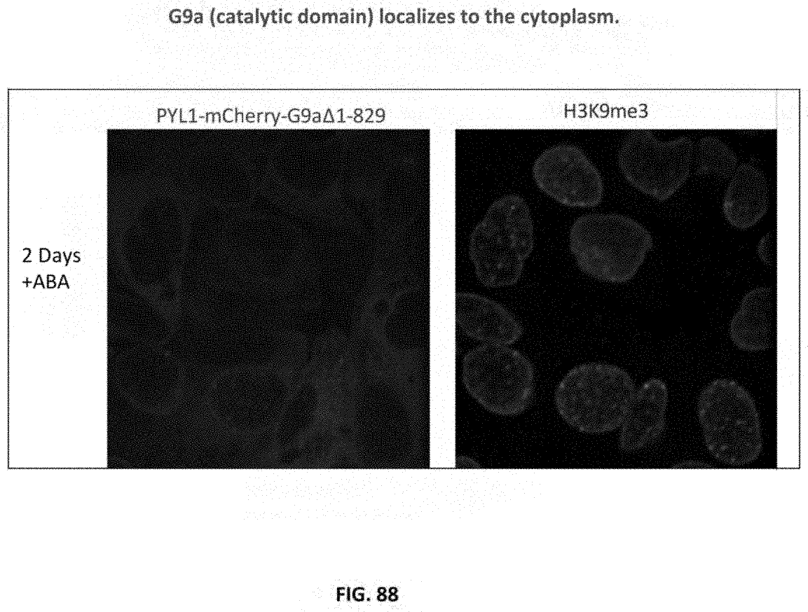

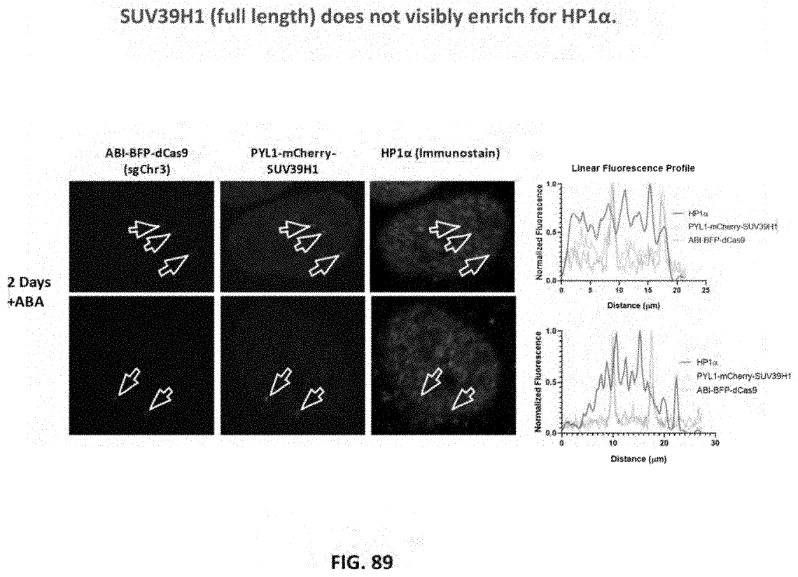

Lastly, catalytic domain of G9a alone was tested, in which the first 829 amino acids were deleted. This variant has previously been shown in vitro to retain enzymatic activity. However, removal of the first 829 amino acids of G9a caused the fusion protein to localize to the cytoplasm instead of nucleus, likely due to removal of the native nuclear localization sequence within G9a. FIG. 88 shows that G9a (catalytic domain; G9aΔ1-829) localized to the cytoplasm. FIG. 88 shows representative fluorescence images taken 2 days after ABA treatment, depicting cells immunostained with H3K9me3 (right panel) localized to the synthetic heterochromatin foci formed by PYL1-mCherry-G9aΔ1-829 (left panel).

Despite enriching for H3K9me3 marks at the Chr3q29 target locus, recruitment of the full length SUV39H1 protein did not visibly enrich for endogenous HP1α at the target locus as assayed by immunostaining. FIG. 89 shows that SUV39H1 (full length) does not visibly enrich for HP1α. FIG. 89 shows representative fluorescence images taken 2 days after ABA treatment, depicting cells immunostained with HP1α (third panel, top and bottom) and synthetic heterochromatin foci formed by PYL1-mCherry-SUV39H1 (second panel, top and bottom) and ABI-BFP-dCas9 (first panel, top and bottom). Line and puncta tracing was used for the corresponding normalized linear fluorescence profile of the co-localization of the HP1α with synthetic heterochromatin puncta, which showed no selective enrichment of HP1α at peaks of PYL1-mCherry-SUV39H1 and ABI-BFP-dCas9 (graphs, top and bottom).

### REFERENCES

Ballister, E. R., Aonbangkhen, C., Mayo, A. M., Lampson, M. A., and Chenoweth, D. M. (2014). Localized light-induced protein dimerization in living cells using a photocaged dimerizer. Nat Commun 5, 5475.
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709-1712.
Benson, G. (1999). Tandem repeats finder: a program to analyze DNA sequences. Nucleic Acids Res 27, 573-580.
Berk, J. M., Tifft, K. E., and Wilson, K. L. (2013). The nuclear envelope LEM-domain protein emerin. Nucleus 4, 298-314.
Bernardi, R., and Pandolfi, P. P. (2003). Role of PML and the PML-nuclear body in the control of programmed cell death. Oncogene 22, 9048-9057.
Bickmore, W. A. (2013). The spatial organization of the human genome. Annu Rev Genomics Hum Genet 14, 67-84.
Bonev, B., and Cavalli, G. (2016). Organization and function of the 3D genome. Nat Rev Genet 17, 772.
Chen, B., Gilbert, L. A., Cimini, B. A., Schnitzbauer, J., Zhang, W., Li, G. W., Park, J., Blackburn, E. H., Weissman, J. S., Qi, L. S., et al. (2013). Dynamic imaging of genomic loci in living human cells by an optimized CRISPR/Cas system. Cell 155, 1479-1491.
Cheutin, T., McNairn, A. J., Jenuwein, T., Gilbert, D. M., Singh, P. B., and Misteli, T. (2003). Maintenance of stable heterochromatin domains by dynamic HP1 binding. Science 299, 721-725.
Clowney, E. J., LeGros, M. A., Mosley, C. P., Clowney, F. G., Markenskoff-Papadimitriou, E. C., Myllys, M., Barnea, G., Larabell, C. A., and Lomvardas, S. (2012). Nuclear aggregation of olfactory receptor genes governs their monogenic expression. Cell 151, 724-737.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
Crabbe, L., Cesare, A. J., Kasuboski, J. M., Fitzpatrick, J. A., and Karlseder, J. (2012). Human telomeres are tethered to the nuclear envelope during postmitotic nuclear assembly. Cell Rep 2, 1521-1529.
Cristofari, G., Adolf, E., Reichenbach, P., Sikora, K., Terns, R. M., Terns, M. P., and Lingner, J. (2007). Human telomerase RNA accumulation in Cajal bodies facilitates telomerase recruitment to telomeres and telomere elongation. Mol Cell 27, 882-889.
de Koning, A. P., Gu, W., Castoe, T. A., Batzer, M. A., and Pollock, D. D. (2011). Repetitive elements may comprise over two-thirds of the human genome. PLoS Genet 7, e1002384.
Dekker, J., Marti-Renom, M. A., and Mirny, L. A. (2013). Exploring the three-dimensional organization of genomes: interpreting chromatin interaction data. Nat Rev Genet 14, 390-403.
Dekker, J., Rippe, K., Dekker, M., and Kleckner, N. (2002). Capturing chromosome conformation. Science 295, 1306-1311.
Denker, A., and de Laat, W. (2016). The second decade of 3C technologies: detailed insights into nuclear organization. Genes Dev 30, 1357-1382.
Finlan, L. E., Sproul, D., Thomson, I., Boyle, S., Kerr, E., Perry, P., Ylstra, B., Chubb, J. R., and Bickmore, W. A. (2008). Recruitment to the nuclear periphery can alter expression of genes in human cells. PLoS Genet 4, e1000039.
Gall, J. G. (2000). Cajal bodies: the first 100 years. Annu Rev Cell Dev Biol 16, 273-300.
Gao, Y., Xiong, X., Wong, S., Charles, E. J., Lim, W. A., and Qi, L. S. (2016). Complex transcriptional modulation with orthogonal and inducible dCas9 regulators. Nat Methods 13, 1043-1049.
Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661.
Grimm, J. B., English, B. P., Chen, J., Slaughter, J. P., Zhang, Z., Revyakin, A., Patel, R., Macklin, J. J., Normanno, D., Singer, R. H., et al. (2015). A general method to improve fluorophores for live-cell and single-molecule microscopy. Nat Methods 12, 244-250, 243 p following 250.
Guan, J., Liu, H., Shi, X., Feng, S., and Huang, B. (2017). Tracking Multiple Genomic Elements Using Correlative CRISPR Imaging and Sequential DNA FISH. Biophys J 112, 1077-1084.
Hilton, L B., D'Ippolito, A. M., Vockley, C. M., Thakore, P. I., Crawford, G. E., Reddy, T. E., and Gersbach, C. A. (2015). Epigenome editing by a CRISPR-Cas9-based acetyltransferase activates genes from promoters and enhancers. Nat Biotechnol 33, 510-517.
Jady, B. E., Richard, P., Bertrand, E., and Kiss, T. (2006). Cell cycle-dependent recruitment of telomerase RNA and Cajal bodies to human telomeres. Mol Biol Cell 17, 944954.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., East, A., Cheng, A., Lin, S., Ma, E., and Doudna, J. (2013). RNA-programmed genome editing in human cells. Elife 2, e00471.
Kaiser, T. E., Intine, R. V., and Dundr, M. (2008). De novo formation of a subnuclear body. Science 322, 1713-1717.
Kearns, N. A., Pham, H., Tabak, B., Genga, R. M., Silverstein, N.J., Garber, M., and Maehr, R. (2015). Functional annotation of native enhancers with a Cas9-histone demethylase fusion. Nat Methods 12, 401-403.
Knight, S. C., Xie, L., Deng, W., Guglielmi, B., Witkowsky, L. B., Bosanac, L., Zhang, E. T., El Beheiry, M., Masson, J. B., Dahan, M., et al. (2015). Dynamics of CRISPR-Cas9 genome interrogation in living cells. Science 350, 823-826.
Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
Kosak, S. T., Skok, J. A., Medina, K. L., Riblet, R., Le Beau, M. M., Fisher, A. G., and Singh, H. (2002). Subnuclear compartmentalization of immunoglobulin loci during lymphocyte development. Science 296, 158-162.
Kumaran, R. I., and Spector, D. L. (2008). A genetic locus targeted to the nuclear periphery in living cells maintains its transcriptional competence. J Cell Biol 180, 51-65.
Langer-Safer, P. R., Levine, M., and Ward, D. C. (1982). Immunological method for mapping genes on Drosophila polytene chromosomes. Proc Natl Acad Sci USA 79, 4381-4385.
Levine, M., Cattoglio, C., and Tjian, R. (2014). Looping back to leap forward: transcription enters a new era. Cell 157, 13-25.
Liang, F. S., Ho, W. Q., and Crabtree, G. R. (2011). Engineering the ABA plant stress pathway for regulation of induced proximity. Sci Signal 4, rs2.
Ma, H., Tu, L. C., Naseri, A., Huisman, M., Zhang, S., Grunwald, D., and Pederson, T. (2016). Multiplexed labeling of genomic loci with dCas9 and engineered sgRNAs using CRISPRainbow. Nat Biotechnol 34, 528-530.
Machyna, M., Neugebauer, K. M., and Stanek, D. (2015). Coilin: The first 25 years. RNA Biol 12, 590-596.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Mao, Y. S., Zhang, B., and Spector, D. L. (2011). Biogenesis and function of nuclear bodies. Trends Genet 27, 295-306.
Morgan, S. L., Mariano, N.C., Bermudez, A., Arruda, N. L., Wu, F., Luo, Y., Shankar, G., Jia, L., Chen, H., Hu, J. F., et al. (2017). Manipulation of nuclear architecture through CRISPR-mediated chromosomal looping. Nat Commun 8, 15993.
Neugebauer, K. M. (2017). Special focus on the Cajal Body. RNA Biol 14, 669-670.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Reddy, K. L., Zullo, J. M., Bertolino, E., and Singh, H. (2008). Transcriptional repression mediated by repositioning of genes to the nuclear lamina. Nature 452, 243-247.
Schmitt, A. D., Hu, M., and Ren, B. (2016). Genome-wide mapping and analysis of chromosome architecture. Nat Rev Mol Cell Biol 17, 743-755.
Schreer, A., Tinson, C., Sherry, J. P., and Schirmer, K. (2005). Application of Alamar blue/5-carboxyfluorescein diacetate acetoxymethyl ester as a noninvasive cell viability assay in primary hepatocytes from rainbow trout. Anal Biochem 344, 76-85.
Shachar, S., Voss, T. C., Pegoraro, G., Sciascia, N., and Misteli, T. (2015). Identification of Gene Positioning Factors Using High-Throughput Imaging Mapping. Cell 162, 911-923.
Shevtsov, S. P., and Dundr, M. (2011). Nucleation of nuclear bodies by RNA. Nat Cell Biol 13, 167-173.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Smogorzewska, A., and de Lange, T. (2002). Different telomere damage signaling pathways in human and mouse cells. EMBO J 21, 4338-4348.
Smoyer, C. J., and Jaspersen, S. L. (2014). Breaking down the wall: the nuclear envelope during mitosis. Curr Opin Cell Biol 26, 1-9.
Takei, Y., Shah, S., Harvey, S., Qi, L. S., and Cai, L. (2017). Multiplexed Dynamic Imaging of Genomic Loci by Combined CRISPR Imaging and DNA Sequential FISH. Biophys J 112, 1773-1776.
Tanenbaum, M. E., Gilbert, L. A., Qi, L. S., Weissman, J. S., and Vale, R. D. (2014). A protein-tagging system for signal amplification in gene expression and fluorescence imaging. Cell 159, 635-646.
Tinevez, J. Y., Perry, N., Schindelin, J., Hoopes, G. M., Reynolds, G. D., Laplantine, E., Bednarek, S. Y., Shorte, S. L., and Eliceiri, K. W. (2017). TrackMate: An open and extensible platform for single-particle tracking. Methods 115, 80-90.
Tsuchiya, Y., Hase, A., Ogawa, M., Yorifuji, H., and Arahata, K. (1999). Distinct regions specify the nuclear membrane targeting of emerin, the responsible protein for Emery-Dreifuss muscular dystrophy. Eur J Biochem 259, 859-865.
van Steensel, B., and Belmont, A. S. (2017). Lamina-Associated Domains: Links with Chromosome Architecture, Heterochromatin, and Gene Repression. Cell 169, 780-791.
Vernier, M., Bourdeau, V., Gaumont-Leclerc, M. F., Moiseeva, O., Begin, V., Saad, F., Mes-Masson, A. M., and Ferbeyre, G. (2011). Regulation of E2Fs and senescence by PML nuclear bodies. Genes Dev 25, 41-50.
Wang, Q., Sawyer, I. A., Sung, M. H., Sturgill, D., Shevtsov, S. P., Pegoraro, G., Hakim, O., Baek, S., Hager, G. L., and Dundr, M. (2016). Cajal bodies are linked to genome conformation. Nat Commun 7, 10966.
Williams, R. R., Azuara, V., Perry, P., Sauer, S., Dvorkina, M., Jorgensen, H., Roix, J., McQueen, P., Misteli, T., Merkenschlager, M., et al. (2006). Neural induction promotes large-scale chromatin reorganisation of the Mashl locus. J Cell Sci 119, 132-140.
Yu, M., and Ren, B. (2017). The Three-Dimensional Organization of Mammalian Genomes. Annu Rev Cell Dev Biol 33, 265-289.
Zhu, L., and Brangwynne, C. P. (2015). Nuclear bodies: the emerging biophysics of nucleoplasmic phases. Curr Opin Cell Biol 34, 23-30.
Zuleger, N., Kelly, D. A., Richardson, A. C., Kerr, A. R., Goldberg, M. W., Goryachev, A. B., and Schirmer, E. C. (2011). System analysis shows distinct mechanisms and common principles of nuclear envelope protein dynamics. J Cell Biol 193, 109-123.
Zullo, J. M., Demarco, I. A., Pique-Regi, R., Gaffney, D. J., Epstein, C. B., Spooner, C. J., Luperchio, T. R., Bernstein, B. E., Pritchard, J. K., Reddy, K. L., et al. (2012). DNA sequence-dependent compartmentalization and silencing of chromatin at the nuclear lamina. Cell 149, 1474-1487.

1-163. (canceled)

164. A system comprising:
a compartmentalization moiety linked to a first coupling domain; and
an actuator moiety linked to a scaffold, wherein the actuator moiety is for binding to a target polynucleotide, and wherein the scaffold is linked to a second coupling domain,
wherein coupling of the first coupling domain to the second coupling domain effects formation of a compartment comprising the compartmentalization moiety adjacent to the target polynucleotide.

165. The system of claim 164, wherein the target polynucleotide is not endogenous to the compartment.

166. The system of claim 164, wherein the scaffold is linked to a plurality of the second coupling domain.

167. The system of claim 166, wherein the scaffold is linked to three or more of the second coupling domain.

168. The system of claim 164, wherein the scaffold comprises a repeating peptide array.

169. The system of claim 164, wherein the scaffold comprises one or more members selected from the group consisting of SpyTag, SunTag, split sfGFP, MoonTag, SnoopTag, split sfCherry2, or mNeonGreen2.

170. The system of claim 164, further comprising a ligand, wherein the ligand binds the first coupling domain and the second coupling domain, to induce the coupling.

171. The system of claim 164, wherein the compartment regulates expression of the target polynucleotide.

172. The system of claim 164, wherein the compartment comprises one or more synthetic heterochromatin foci.

173. The system of claim 164, wherein the compartmentalization moiety comprises at least a portion of a nuclear heterochromatin protein.

174. The system of claim 173, wherein the nuclear heterochromatin protein is selected from the group consisting of HP1α, HP1β, KAP1, KRAB, SUV39H1, and G9a.

175. The system of claim 174, wherein the nuclear heterochromatin protein is HP1α.

176. The system of claim 164, wherein the target polynucleotide comprises a genomic polynucleotide sequence.

177. The system of claim 164, wherein the target polynucleotide comprises a non-coding polynucleotide sequence.

178. The system of claim 164, wherein the actuator moiety comprises a Cas protein, and wherein the system further comprises a guide RNA that complexes with the actuator moiety and hybridizes to the target polynucleotide.

179. A method comprising:
(a) providing (i) a compartmentalization moiety linked to a first coupling domain, and (ii) an actuator moiety linked to a scaffold, wherein the actuator moiety is for binding to a target polynucleotide, and wherein the scaffold is linked to a second coupling domain; and
(b) coupling the first coupling domain to the second coupling domain, to form a compartment comprising the compartmentalization moiety adjacent to the target polynucleotide.

180. The method of claim 179, wherein the target polynucleotide is not endogenous to the compartment.

181. The method of claim 179, wherein the scaffold is linked to a plurality of the second coupling domain.

182. The method of claim 179, wherein the scaffold comprises a repeating peptide array.

183. The method of claim 179, further comprising using a ligand to couple the first coupling domain to the second coupling domain, wherein the ligand is capable of binding to the first coupling domain and to the second coupling domain.
